(19) United States
(12) Patent Application Publication
(10) Pub. No.: US 2025/0109121 A1
(43) Pub. Date: Apr. 3, 2025

(54) POTENT AND SELECTIVE SMARCA2 DEGRADING CHIMERIC MOLECULES AS CANCER THERAPEUTICS

(71) Applicant: BOARD OF REGENTS, THE UNIVERSITY OF TEXAS SYSTEM, Austin, TX (US)

(72) Inventor: Yonathan LISSANU, Houston, TX (US)

(21) Appl. No.: 18/725,495

(22) PCT Filed: Dec. 23, 2022
(86) PCT No.: PCT/US2022/053950

§ 371 Date: Jun. 28, 2024

Related U.S. Application Data
(60) Provisional application No. 63/266,068, filed on Dec. 28, 2021.

(51) Int. Cl. C07D 401/14; A61K 31/501; A61K 45/06; A61K 47/55; A61P 35/00

(52) U.S. Cl. CPC C07D 401/14; A61K 31/501; A61K 45/06; A61K 47/55; A61P 35/00

(57) ABSTRACT

Provided are heterobifunctional compounds for the treatment of SMARCA4 deficient cancers, which heterobifunctional compounds comprise a SMARCA2 binding moiety, a linker, and a ubiquitin ligase binding moiety.

### CROSS-REFERENCE TO RELATED APPLICATIONS

This application claims the benefit of U.S. Provisional Application No. 63/266,068, filed Dec. 28, 2021, which is incorporated by reference herein in its entirety.

### STATEMENT REGARDING FEDERALLY SPONSORED RESEARCH OR DEVELOPMENT

This invention was made with government support under CA272945 awarded by the National Institutes of Health. The government has certain rights in the invention.

### BACKGROUND

Switch/Sucrose Non Fermentable (SWI/SNF) is a multi-subunit complex that modulates chromatic structure through the activity of two mutually exclusive helicase/ATPase catalytic subunits: SWI/SNF-Related, Matrix-Associated, Actin-Dependent Regulator of Chromatin, Subfamily A, Member 2 (SMARCA2) and SWI/SNF-Related, Matrix-Associated, Actin-Dependent Regulator of Chromatin, Subfamily A, Member 4 (SMARCA4). The core and the regulatory subunits of SMARCA2 and SMARCA4 couple ATP hydrolysis to the perturbation of histone-DNA contacts, thereby providing access points to transcription factors and cognate DNA elements that facilitate gene activation and repression.

Despite having a high degree of homology, and their presumed overlapping functions, SMARCA2 and SMARCA4 have been reported as having different roles in cancer. For example, SMARCA4 is frequently mutated in primary tumors, while SMARCA2 inactivation is infrequent in tumor development. In fact, numerous types of cancer have been shown to be SMARCA4-related, e.g., cancers having a SMARCA4 mutation or a SMARCA4 deficiency, including, e.g, non-small cell lung cancers.

SMARCA2 has been demonstrated as an essential gene in SMARCA4-deficient or SMARCA4 mutant cancer cells because SMARCA4-deficient or -mutant cells depend exclusively on SMARCA2 activity. In these cells, incorporation of SMARCA2 into the complex is increased to compensate for the SMARCA4 deficiency. Therefore, SMARCA2 may be targeted in SMARCA4-deficient/mutant cancers. However, identifying selective inhibitors of SMARCA2 has been challenging because SMARCA2 and SMARCA4 have highly similar bromodomains and ATPase domains. Thus, there is a need for selective inhibitors of SMARCA2.

### BRIEF SUMMARY

In some aspects, provided is a compound of Formula I:

D-L-S
or a pharmaceutically acceptable salt thereof,
wherein:
   D is a ubiquitin ligase binding moiety,
   L is a linker comprising a fluoro-phenyl or difluoro-phenyl substituted with one or more groups selected from the group consisting of a piperazine group, a piperidine group, methoxypropyl and a methoxyethoxy group, and
   S is a SMARCA2 binding moiety.

In some aspects, provided is a compound of Formula I:

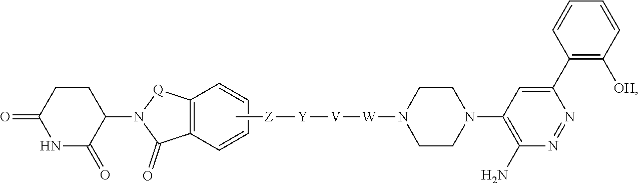

or a pharmaceutically acceptable salt or solvate thereof, wherein:
Q is selected from the group consisting of —CH₂— and —C(═O)—;
W is —CH₂—;
V is:

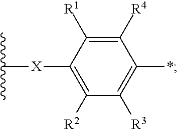

wherein the bond marked with an “*” is attached to W;
R¹, R², R³, and R⁴ are independently selected from the group consisting of hydrogen and fluoro;
X is selected from the group consisting of —O—, N(R^{5a})—

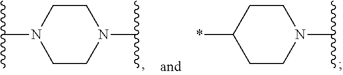 [SMILES: *N1CCN(*)CC1 ; *C1CCN(*)CC1]

wherein the bond marked with an “*” is attached to Y; or
X is a bond;
R^{5a} is selected from the group consisting of hydrogen and C₁-C₄ alkyl;
Y is selected from the group consisting of C₁-C₈ alkylenyl and 3- to 8-membered heteroalkylenyl; or
Y is a bond; and
Z is selected from the group consisting of —O—, —N(R^{5b})—,

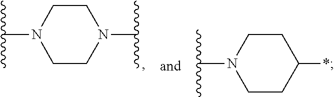

wherein the bond marked with an “*” is attached to Y; or
Z is a bond; and
R^{5b} is selected from the group consisting of hydrogen and C₁-C₄ alkyl.

In some aspects, provided is a compound of Formula III:

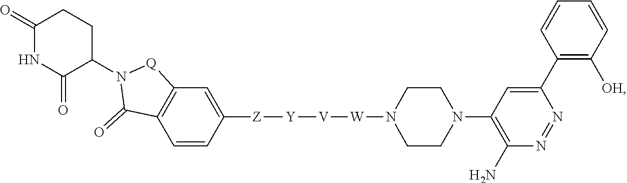

or a pharmaceutically acceptable salt or solvate thereof.

In some aspects, provided is a compound of Formula IV:

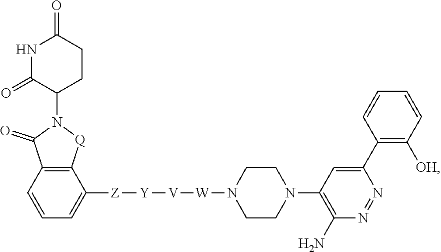

or a pharmaceutically acceptable salt or solvate thereof.

In some aspects, Q is —C(═O)— in a compound of Formula I-IV, or a pharmaceutically acceptable salt or solvate thereof,

In some aspects, R¹ is fluoro, and R², R³, and R⁴ are hydrogen.

In some aspects. R⁴ is fluoro, and R¹, R², and R³ are hydrogen.

In some aspects, R¹ and R² are fluoro, and R³ and R⁴ are hydrogen.

In some aspects, R¹ and R⁴ are fluoro, and R² and R³ are hydrogen.

In some aspects, R¹ and R³ are fluoro, and R² and R⁴ are hydrogen.

In some aspects, R³ and R⁴ are fluoro, and R¹ and R² are hydrogen.

In some aspects, X is a bond. In some aspects, X is —O—.

In some aspects, Y is C₁-C₈ alkylenyl. In some aspects, Y is selected from the group consisting of —CH₂—, —CH₂CH₂—, —CH₂CH₂CH₂— and —CH₂CH₂CH₂CH₂—. In some aspects, Y is 3- to 8-membered heteroalkylenyl. In some aspects, Y is selected from the group consisting of —CH₂CH₂OCH₂CH₂— and —CH₂CH₂OCH₂CH₂CH₂—.

In some aspects, Z is of —O—. In some aspects, Z is of —N(R^{5b})—.

In some aspects, —Z—Y—V—W— is selected from the group consisting of

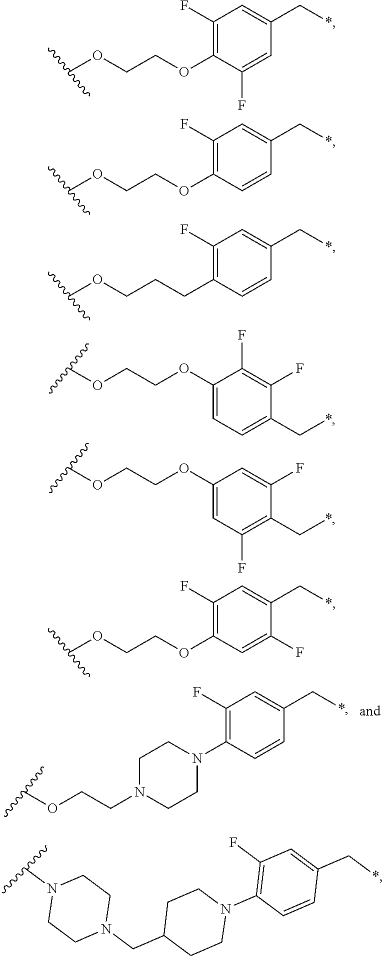

wherein the bond marked with an “*” is attached to the SMARCA2 binding moiety.

In some aspects, the compound is selected from the group consisting of:

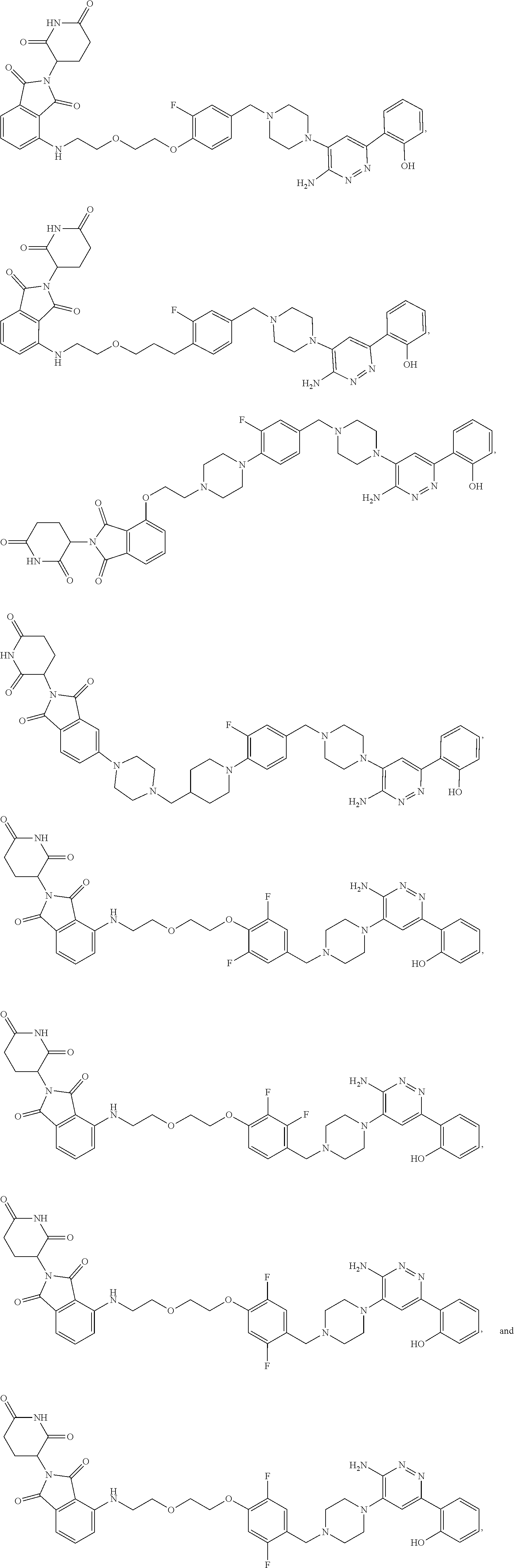

or a pharmaceutically acceptable salt thereof.

In some aspects, the compound of the disclosure has a DC₅₀ (concentration at which 50% of the target protein is degraded) for SMARCA2 of about 0.1 nM to about 300 nM, about 1 nM to about 200 nM, about 2 nM to about 150 nM, about 5 nM to about 120 nM, about 10 nM to about 100 nM, about 15 nM to about 80 nM, about 20 nM to about 70 nM, or about 30 nM to about 60 nM.

In some aspects, the compound of the disclosure has a DC₅₀ for SMARCA2 of less than about 300 nM, less than about 200 nM, less than about 150 nM, less than about 120 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, or less than about 2 nM.

In some aspects, the compound of the disclosure has a Dₘₐₓ (maximum degradation as percentage of DMSO control) for SMARCA2 of about 60% to about 100%, about 65% to about 99%, about 70% to about 95%, about 75% to about 90%.

In some aspects, the compound of the disclosure has a Dₘₐₓ for SMARCA2 of about 60%, 65%, 67%, 70%, 72%, 75%, 77%, 80%, 82%, 85%, 87%, 90%, 92%, 95%, 97%, 98%, 99%, or 100%.

In some aspects, the compound of the disclosure has a DC₅₀ for SMARCA4 of about 500 nM to about 100 μM, about 700 nM to about 90 μM, about 800 nM to about 80 μM, about 900 nM to about 70 μM, about 1 M to about 60 μM, about 2 M to about 50 μM, about 5 M to about 25 μM, or about 10 μM to about 20 μM.

In some aspects, the compound of the disclosure has a DC₅₀ for SMARCA4 of more than about 500 nM, more than about 700 nM, more than about 1 μM, more than about 2 μM, more than about 5 μM, more than about 7 μM, or more than about 10 PM.

In some aspects, the compound of the disclosure has a Dₘₐₓ for SMARCA4 of about 10% to about 80%, about 15% to about 70%, about 20% to about 60%, or about 25% to about 50%.

In some aspects, the compound of the disclosure has a Dₘₐₓ for SMARCA4 of less than about 80%, less than about 70%, less than about 60%, less than about 50%, less than about 40%, less than about 25% or less than about 20%.

In some aspects, the compound of the disclosure has an IC₅₀ (concentration at which 50% of cellular proliferation is inhibited) of about 10 nM to about 50 μM, about 50 nM to about 20 μM, about 100 nM to about 10 μM, about 200 nM to about 8 μM, about 300 nM to about 5 μM, about 400 nM to about 2 μM, or about 500 nM to about 1 μM.

In some aspects, the compound of the disclosure has an IC₅₀ in wild-type SMARCA4 cells of about 900 nM to about 50 μM, about 1 M to about 40 μM, about 2 M to about 30 μM, about 5 μM to about 25 μM, or about 10 μM to about 20 μM.

In some aspects, the compound of the disclosure has an IC₅₀ in wild-type SMARCA4 cells of more than about 900 nM, more than about 1 μM, more than about 2 μM, more than about 3 μM, more than about 5 μM, more than about 7 μM, or more than about 10 μM.

In some aspects, the compound of the disclosure has an IC₅₀ in mutant SMARCA4 cells of about 50 nM to about 10 μM, about 75 nM to about 5 μM, about 100 nM to about 1 μM, about 125 nM to about 900 nM, about 150 nM to about 800 nM, about 175 nM to about 700 nM, about 200 nM to about 600 nM, about 250 nM to about 500 nM.

In some aspects, the compound of the disclosure has an IC₅₀ in mutant SMARCA4 cells of less than about 10 μM, less than about 5 μM, less than about 2 μM, less than about 1 μM, less than about 900 nM, less than about 800 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, or less than about 2 nM, or less than about 1 nM.

In some aspects, the compound of the disclosure competitively inhibits binding of pomalidomide to cereblon.

In some aspects, the compound of the disclosure competitively inhibits binding of a SMARCA2 inhibitor to SMARCA2.

In some aspects, the compound of the disclosure inhibits proliferation of SMARCA4 mutant cancer cells.

In some aspects, the compound of the disclosure does not inhibit proliferation of SMARCA4 wild-type cells.

In some aspects, provided is a pharmaceutical composition comprising a compound of the disclosure, or a pharmaceutically acceptable salt thereof, and a pharmaceutically acceptable adjuvant, carrier, or vehicle.

In some aspects, the pharmaceutical composition further comprises an additional pharmaceutically active agent.

In some aspects, the additional pharmaceutically active agent is a hormone analogue or antihormone, an aromatase inhibitor, a growth factor inhibitor, an antimetabolite, an antitumor antibiotic, a platinum derivative, an antimitotic agent, an angiogenesis inhibitor, a topoisomerase inhibitor, a serine/threonine kinase inhibitor, a tyrosine kinase inhibitor, a PARP inhibitor, a tubulin inhibitor, a DNA synthesis inhibitor a protein-protein interaction inhibitor, a MEK/ERK inhibitor, a TRAIL inhibitor, a BCR-ABL inhibitor, a HDAC inhibitor, a radiopharmaceutical, an immune checkpoint inhibitor, an ADCC enhancer, a T cell engager, a chemotherapeutic agent, or a tumor vaccine.

In some aspects, provided is a method of inducing degradation of a SMARCA2 in a subject in need thereof, comprising administering to the subject a therapeutically effective amount of a pharmaceutical composition as described herein.

In some aspects, the subject suffers from a SMARCA4 deficient malignancy.

In some aspects, the SMARCA4 deficient malignancy comprises a SMARCA4 inactivating mutation or a SMARCA4 deletion.

In some aspects, the SMARCA4 deletion is a SMARCA4 gene truncation.

In some aspects, the SMARCA4 deficient malignancy is a lung cancer, a liver cancer, a bladder cancer, a breast cancer or a colon cancer.

In some aspects, the lung cancer is non-small cell lung cancer.

In some aspects, provided is a method of treating a SMARCA4 deficient malignancy in a subject in need thereof, comprising administering to the subject a therapeutically effective amount of a pharmaceutical composition described herein.

In some aspects, provided is a method of treating a tumor in a subject in need thereof, comprising administering to the subject a therapeutically effective amount of the pharmaceutical composition described herein.

In some aspects, the tumor comprises a SMARCA4 inactivating mutation or deletion.

In some aspects, the tumor is a lung cancer, a liver cancer, a bladder cancer, a breast cancer or a colon cancer.

In some aspects, the lung cancer is non-small cell lung cancer.

In some aspects, the method further comprises administering a hormone analogue or antihormone, an aromatase inhibitor, a growth factor inhibitor, an antimetabolite, an antitumor antibiotic, a platinum derivative, an antimitotic agent, an angiogenesis inhibitor, a topoisomerase inhibitor, a serine/threonine kinase inhibitor, a tyrosine kinase inhibitor, a PARP inhibitor, a tubulin inhibitor, a DNA synthesis inhibitor a protein-protein interaction inhibitor, a MEK/ERK inhibitor, a TRAIL inhibitor, a BCR-ABL inhibitor, a HDAC inhibitor, a radiopharmaceutical, an immune checkpoint inhibitor, an ADCC enhancer, a T cell engager, a chemotherapeutic agent, or a tumor vaccine.

In some aspects, provided is a method of treating a condition or disease selected from a chronic autoimmune disorder, an inflammatory condition, a proliferative disorder, a sepsis, or a viral infection, comprising administering to a subject in need thereof a therapeutically effective amount of the pharmaceutical composition described herein.

In some aspects, provided is a heterobifunctional compound comprising two binding moieties connected by a linker, wherein the linker is selected from the group consisting of:

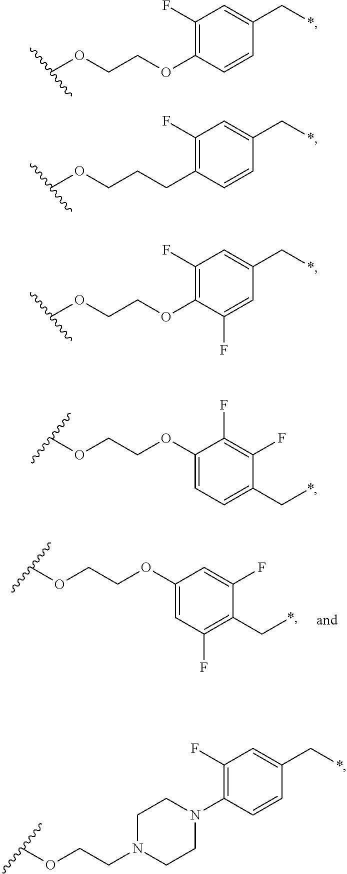

wherein the bond marked with an “*” is attached to the SMARCA2 binding moiety.

In some aspects, the linker is seleted from

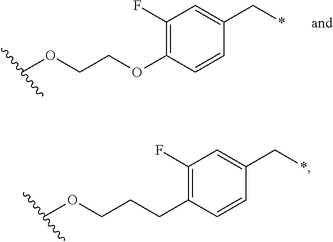

wherein the bond marked with an “*” is attached to the SMARCA2 binding moiety.

### BRIEF DESCRIPTION OF THE DRAWINGS

FIG. 1 shows a schematic of a heterobifunctional compound interacting with a SMARCA2 protein and a cereblon ubiquitin ligase.

FIG. 2A shows degradation profiles of SMARCA2 by SMARCA2 PROTAC Compound 101 and ACBI1 in different cell lines with wild-type SMARCA4. FIG. 2B shows degradation profiles of SMARCA4 by SMARCA2 PROTAC Compound 101 and ACBI1 in different cell lines with wild-type SMARCA4.

FIG. 3A shows a colony assay with cell lines expressing wild-type SMARCA4 stained with crystal violet after treatment with DMSO or different concentrations of SMARCA2 PROTAC Compound 101 and ACBI1. FIG. 3B shows a colony assay with cell lines expressing mutant SMARCA4 stained with crystal violet after treatment with DMSO or different concentrations of a SMARCA2 PROTAC Compound 101 and ACBI1. FIG. 3C shows a line graph of the colonies of different wild-type SMARCA4 cell lines (dashed lines) and mutant SMARCA4 cell lines (solid lines) after treatment with ACBI1. FIG. 3D shows a line graph of the colonies of different wild-type SMARCA4 cell lines (dashed lines) and mutant SMARCA4 cell lines (solid lines) after treatment with the SMARCA2 PROTAC Compound 101.

FIG. 4A shows western blots of SMARCA2 expression in H1792 cells (wild-type SMARCA4) and H1299 cells (mutant SMARCA4) treated for 72 hours or 96 hours with different concentrations of SMARCA2 PROTAC Compound 103. FIG. 4B shows western blots of SMARCA4 expression in H1792 cells and H1299 cells treated for 72 hours or 96 hours with different concentrations of SMARCA2 PROTAC Compound 103. FIG. 4C shows western blots of Polybromo 1 (PBRM1) expression in H1792 cells and H1299 cells treated for 72 hours or 96 hours with different concentrations of SMARCA2 PROTAC Compound 103.

FIG. 5A shows western blots of SMARCA2 expression in H1792 cells (wild-type SMARCA4) and H1299 cells (mutant SMARCA4) treated for 72 hours or 96 hours with different concentrations of SMARCA2 PROTAC Compound 102. FIG. 5B shows western blots of SMARCA4 expression in H1792 cells and H1299 cells treated for 72 hours or 96 hours with different concentrations of SMARCA2 PROTAC Compound 102. FIG. 5C shows western blots of Polybromo 1 (PBRM1) expression in H1792 cells and H1299 cells treated for 72 hours or 96 hours with different concentrations of SMARCA2 PROTAC Compound 102.

FIG. 6A shows western blots of SMARCA2, SMARCA4, and PBRM1 expression in H1975 cells treated with different concentrations of Compound 104 and Compound 105. FIG. 6B shows western blots of SMARCA2, SMARCA4, and PBRM1 expression in H1975 cells treated with different concentrations of Compound 106, Compound 107, and Compound 108.

FIG. 7A shows immunohistochemistry images of SMARCA2 in H1299 xenograft tumors of female NCR Nude mice treated with vehicle or the SMARCA2 degrader Compound 101 (200 mg/kg) intraperitoneally. FIG. 7B shows a quantification of SMARCA2 protein levels in the xenografts of mice treated with vehicle or the SMARCA2 degrader Compound 101 at 200 mg/kg concentration intraperitoneally for four days and tumor tissue harvested 24 hours after the last dose.

FIG. 8 shows xenograft tumor growth measurements of female NCR nude mice implanted with HCC515 lung cancer and then treated with vehicle control or the SMARCA2 degrader Compound 101 (12.5 mg/kg) administered intraperitoneally, daily for the indicated duration.

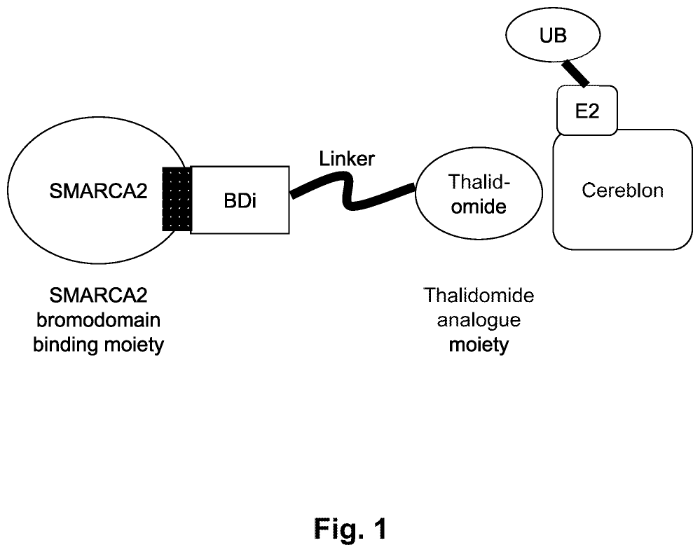

### DETAILED DESCRIPTION

### I. Definitions

Unless defined otherwise, all technical and scientific terms used herein have the same meaning as commonly understood by those skilled in the art.

The singular forms “a,” “an,” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some aspects of the disclosure, the term “about” encompasses a deviation from the recited value of between 0.001% and 10%, inclusive of the endpoints. In some aspects, the term “about” encompasses an increase from the recited value of between 0.001% and 10%, inclusive of the endpoints. In some aspects, the term “about” encompasses a decrease from the recited value of between 0.001% and 10%, inclusive of the endpoints.

Units, prefixes, and symbols are denoted in their Système International de Unites (SI) accepted form.

Numeric ranges are inclusive of the numbers defining the range. Where a range of values is recited, it is to be understood that each intervening integer value, and each fraction thereof, between the recited upper and lower limits of that range is also specifically disclosed, along with each subrange between such values.

The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Any numerical value inherently contains certain errors necessarily resulting from the standard deviation found in their respective measurements. Additionally, all ranges disclosed herein are to be understood to encompass any and all subranges subsumed therein. For example, a stated range “1 to 10” should be considered to include any and all subranges between (and inclusive of) the minimum value of 1 and the maximum value of 10; that is, all subranges beginning with a minimum value of 1 or more, such as 1 to 6.1, and ending with a maximum value of 10 or less, such as 5.5 to 10.

Additionally, any reference recited herein or referred to as being “incorporated herein” is to be understood as being incorporated in its entirety.

Where a value is explicitly recited, it is to be understood that values, which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “heterobifunctional compound” as used herein refers to a chemical compound that is characterized by having different reactive groups at either end of the compound. The groups of the compound may connect through a linker. In some aspects the heterobifunctional compound is represented by Formula I D-L_S, where D is a degrader moiety that is capable of binding a ubiquitin ligase, L is a linker connecting D and S and S is a moiety capable of binding to SMARCA2. In some aspects the heterobifunctional compound is represented by Formula II, III, IV, and/or V. Heterobifunctional compounds represented by Formulae I-V are collectively referred to herein as “compounds of the disclosure.”

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “alkylenyl” as used herein by itself or part of another group refers to a divalent form of an alkyl group. In one embodiment, the alkylenyl is a divalent form of a C₁-C₁₂ alkyl, i.e., a C₁-C₁₂ alkylenyl. In another embodiment, the alkylenyl is a divalent form of a C₁-C₁₀ alkyl, i.e., a C₁-C₁₀ alkylenyl. In another embodiment, the alkylenyl is a divalent form of a C₁-C₈, i.e., a C₁-C₈ alkylenyl. In another embodiment, the alkylenyl is a divalent form of an unsubstituted C₁-C₆ alkyl, i.e., a C₁-C₆ alkylenyl. In another embodiment, the alkylenyl is a divalent form of an unsubstituted C₁₋₄ alkyl, i.e., a C₁-C₄ alkylenyl. Non-limiting exemplary alkylenyl groups include —CH₂—, —CH₂CH₂—, —CH₂CH₂CH₂—, —CH₂(CH₂)₂CH₂—, —CH(CH₂)₃CH₂—, and —CH₂(CH₂)₄CH₂—.

The term “heteroalkyl” as used by itself or part of another group refers to unsubstituted straight- or branched-chain aliphatic hydrocarbons containing from three to 15 chain atoms, i.e., 3- to 15-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. Non-limiting exemplary heteroalkyl groups include —CH₂OCH₃, —CH₂CH₂OCH₂CH₃, —CH₂CH₂N(H)CH₂CH₃, —CH₂OCH₂CH₂CH₃, —CH₂CH₂CH—₂OCH₃, —CH₂CH₂OCH₂CH₂OCH₂CH₃, —CH₂CH₂OCH₂CH₂OCH₂CH₂OCH₂CH₃.

The term “heteroalkylenyl” as used herein by itself or part of another group refers to a divalent form of a heteroalkyl group. In one embodiment, the heteroalkylenyl is a 3- to 15-membered heteroalkylenyl. In another embodiment, the heteroalkylenyl a 3- to 10-membered heteroalkylenyl. In another embodiment, the heteroalkylenyl is a 3- to 8-membered heteroalkylenyl. In another embodiment, the heteroalkylenyl is a 3- to 6-membered heteroalkylenyl. In another embodiment, the heteroalkylenyl is a 5-membered heteroalkylenyl. Non-limiting exemplary heteroalkylenyl groups include —CH₂OCH₂—, —CH₂OCH₂CH₂—, —CH₂CH₂OCH₂CH₂—, —CH₂OCH₂CH₂CH₂—, —CH₂CH₂OCH₂CH₂CH₂—, and —CH₂CH₂OCH₂CH₂OCH₂CH₂—.

The term “degrader moiety” as used herein refers to a compound that binds to and degrades a bromodomain protein with measurable activity.

The term “measurable activity” as used herein refers to a measurable degradation of a bromodomain protein between (i) a sample comprising a compound of Formula I or Formula II, or composition thereof, and such bromodomain protein and (ii) an equivalent sample comprising such bromodomain protein, in the absence of the compound of Formula I or Formula II, or composition thereof.

The term “linker” refers to a molecule that comprises a fluoro-phenyl or difluoro-phenyl substituted with one or more groups selected from a piperazine group, a piperidine group, a methoxypropyl, and a methoxyethoxy group.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin.

The terms “ubiquitin ligase binding moiety” or “moiety capable of binding ubiquitin ligase” as used herein refer to any molecular structure that can bind a ubiquitin ligase enzyme and includes, but is not limited to, thalidominde, pomalidomide, lenalidomide, CC-885, CC-122, CC-220, a bBETI. Examples of ubiquitin ligase binding moieties are disclosed in Ito et al. Proc. Jpn. Acad. Ser B 96 (2020).

The terms “SMARCA2 binding moiety” or “moiety capable of binding to SMARCA2” as used herein refer to a compound that can bind a SMARCA2 bromodomain and inhibit its function. The SMARCA2 bromodomain inhibitor is used as a ligand in the heterobifunctional compound of the disclosure.

The term “pharmaceutical composition” as used herein refers to a preparation which is in such form as to permit the biological activity of the active ingredient, e.g., a compound of the disclosure, to be effective, and which contains no additional components which are unacceptably toxic to a subject to which the composition would be administered. Such compositions can be sterile. A “pharmaceutical composition” comprises a compound according to the invention described herein, or a pharmaceutically acceptable salt thereof, having a desired degree of purity in a physiologically acceptable carrier, excipient or stabilizer. Pharmaceutically acceptable carriers, excipients, or stabilizers are nontoxic to recipients at the dosages and concentrations employed and include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

The term “pharmaceutically acceptable salts” refers to the relatively non-toxic, inorganic and organic acid addition salts. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

The terms “pharmaceutically acceptable carrier,” “pharmaceutically acceptable excipient,” “pharmaceutically acceptable adjuvant,” or “pharmaceutically acceptable vehicle” refer to a pharmaceutically acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The term “administering,” as used herein, refers to any mode of transferring, delivering, introducing, or transporting a therapeutic agent to a subject in need of treatment with such an agent. Such modes of administration include oral, topical, and parenteral administration.

The term “parenteral administration” as used herein includes intravenous, intraperitoneal, intramuscular, intradermal, intranasal, subcutaneous, intraarterial, intracranial, intrathecal, intracapsular, intraorbital, intracardiac, intrahepatic, transtracheal, subcuticular, intraarticular, intrasynovial, subcapsular, subarachnoid, intraspinal, epidural, intrasternal, and intralesional injections and infusions.

The term “therapeutically effective amount” refers to an amount of a compound according to the invention that provides beneficial or desired therapeutic and/or prophylactic results. For prophylactic use, beneficial or desired results can include, for example, one or more results such as eliminating or reducing the risk, lessening the severity, or delaying the onset or recurrence of the disease, including biochemical, histological, and/or behavioral symptoms of the disease, its complications, and intermediate pathological phenotypes presenting during development of the disease. For therapeutic use, beneficial or desired results can include, for example, one or more clinical results such as decreasing one or more symptoms and pathological conditions resulting from or associated with the disease, increasing the quality of life of those suffering from the disease, decreasing the dose of other medications required to treat the disease, enhancing the effect of other medication such as via targeting, delaying the progression of the disease, and/or prolonging survival. An effective amount can be, for example, an amount sufficient to accomplish prophylactic or therapeutic treatment either directly or indirectly. As it is understood in the clinical context, an effective amount of a drug, compound, or pharmaceutical composition may or may not be achieved in conjunction with another drug, compound, or pharmaceutical composition. Thus, an effective amount may be considered in the context of administering one or more therapeutic agents. An effective amount can be administered in one dosage or can be divided into multiple dosages, the total of such dosages being the effective amount. For example, an effective amount can be provided in two separate administrations over a period of time that, in aggregate, provide the effective amount of the formulation.

The terms “tumor cell” or neoplastic cell and grammatical equivalents refer to the total population of cells derived from a tumor, a pre-tumorous lesion, or a neoplasm, including both non-tumorigenic cells, which comprise the bulk of the tumor cell population, and tumorigenic stem cells. As used herein, the term “tumor cell” or “neoplastic cell” will be modified by the term “non-tumorigenic” when referring solely to those tumor cells lacking the capacity to renew and differentiate to distinguish those tumor cells from tumor stem cells.

The terms “tumor” and “neoplasm” as used herein refer to any mass of tissue that result from excessive cell growth or proliferation, either benign (non-tumorous) or malignant (tumorous) including pre-tumorous lesions.

The terms “SMARCA4 deficient malignancy” or “SMARCA4 mutant malignancy” or “SMARCA4 deficient tumor” or “SMARCA4 mutant tumor” or “SMARCA4 deficient cell” or “SMARCA4 mutant cell” refer to a malignancy, tumor or cell in which the SMARCA4 gene is either deleted or mutated or the SMARCA4 protein is functionally deficient. SAMRCA4 deficient or mutant tumors include, e.g., lung cancer, specifically non-small cell lung cancer, liver cancer, bladder cancer, breast cancer, colon cancer, gastrointestinal cancer, melanoma, endometrium cancer or sarcomas.

### II. Heterobifunctional Compounds

The present disclosure describes heterobifunctional compounds, which recruit endogenous proteins to a ubiquitin ligase for degradation, and methods of using the same.

In some aspects, the disclosure provides heterobifunctional or proteolysis targeting chimeric (PROTAC) compounds that contain a ligand for a target protein of interest connected via a linker to a ligand for an E3 ubiquitin ligase.

In some aspects, the disclosure provides a compound of Formula I, or a pharmaceutically acceptable salt or solvate thereof:

D-L-S

which comprises a moiety capable of binding a ubiquitin ligase (i.e., degrader “D”), a linker (“L”), and a moiety capable of binding a target protein, e.g., SMARCA2 (“S”).

In some aspects, the heterobifunctional compound has Formula II:

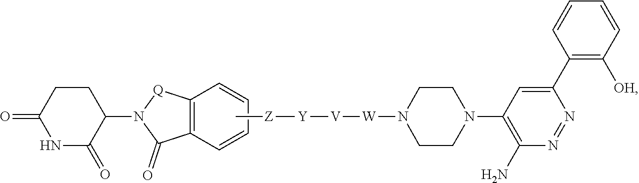

or a pharmaceutically acceptable salt or solvate thereof,

In some aspects, the heterobifunctional compound has Formula III:

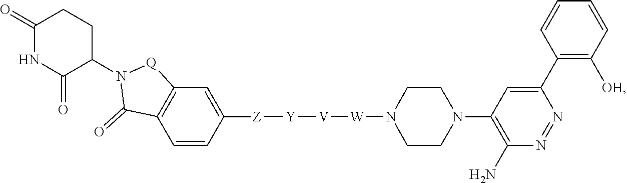

or a pharmaceutically acceptable salt or solvate thereof.

In some aspects, the heterobifunctional compound has Formula IV:

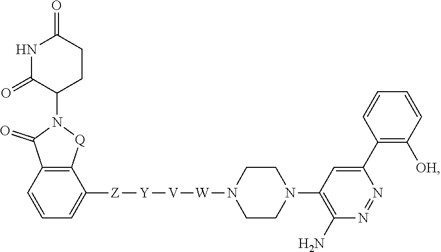

or a pharmaceutically acceptable salt or solvate thereof.

In some aspects, the heterobifunctional compound has Formula V:

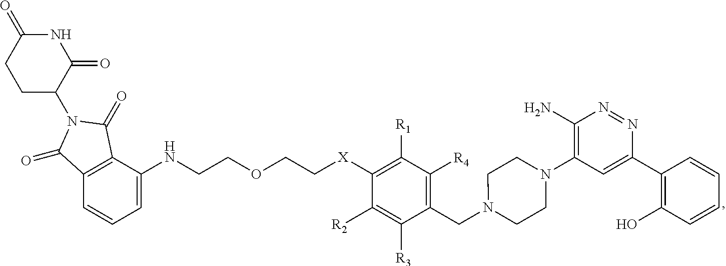

or a pharmaceutically acceptable salt or solvate thereof.

In some aspects, Q is selected from the group consisting of —CH₂— and —C(═O)—; W is —CH₂—; V is:

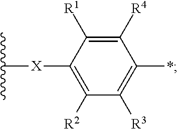

wherein the bond marked with an “*” is attached to W;
R¹, R², R³, and R⁴ are independently selected from the group consisting of hydrogen and fluoro;
X is selected from the group consisting of —O—, N(R^{5a})—

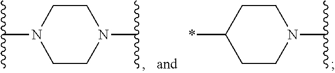 [SMILES: *N1CCN(*)CC1 ; *C1CCN(*)CC1]

wherein the bond marked with an “*” is attached to Y; or
X is a bond;
R^{5a} is selected from the group consisting of hydrogen and C₁-C₄ alkyl;
Y is selected from the group consisting of C₁-C₈ alkylenyl and 3- to 8-membered heteroalkylenyl; or
Y is a bond; and
Z is selected from the group consisting of —O—, —N(R^{5b})—,

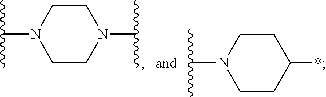

wherein the bond marked with an “*” is attached to Y; or
Z is a bond; and
R^{5b} is selected from the group consisting of hydrogen and C₁-C₄ alkyl.

In some aspects where the heterobifunctional compound has Formula II, III, IV, or V, R¹ is fluoro, and R², R³, and R⁴ are hydrogen. In some aspects where the heterobifunctional compound has Formula II, III, IV, or V, R⁴ is fluoro, and R¹, R², and R³ are hydrogen. In some aspects where the heterobifunctional compound has Formula II, III, IV, or V, R¹ and R² are fluoro, and R³ and R⁴ are hydrogen. In some aspects where the heterobifunctional compound has Formula II, III, IV, or V, R¹ and R⁴ are fluoro, and R² and R³ are hydrogen. In some aspects where the heterobifunctional compound has Formula II, III, IV, or V, R¹ and R³ are fluoro, and R² and R⁴ are hydrogen. In some aspects, where the heterobifunctional compound has Formula II, III, IV, or V, R³ and R⁴ are fluoro, and R¹ and R² are hydrogen.

In some aspects, where the heterobifunctional compound has Formula II, III, IV, or V, X is a bond. In some aspects, where the heterobifunctional compound has Formula II, III, IV, or V, X is —O—.

In some aspects, where the heterobifunctional compound has Formula II, III, IV, or V, Y is C₁-C₈ alkylenyl. In aspects where Y is C₁-C₈ alkylenyl, Y is selected from the group consisting of —CH₂—, —CH₂CH₂—, —CH₂CH₂CH₂— and —CH₂CH₂CH₂CH₂—.

In some aspects, where the heterobifunctional compound has Formula II, III, IV, or V, Y is 3- to 8-membered heteroalkylenyl. In aspects where Y is 3- to 8-membered heteroalkylenyl, Y is selected from the group consisting of —CH₂CH₂OCH₂CH₂— and —CH₂CH₂OCH₂CH₂CH₂—.

In some aspects, where the heterobifunctional compound has Formula II, III, IV, or V, Z is of —O—. In some aspects, where the heterobifunctional compound has Formula II, III, IV, or V, Z is of —N(R^{5b})—.

In some aspects, the heterobifunctional compound has Formula V and X is O, R₁ is fluoride, and R₂, R₃, and R₄ are each hydrogen (“Compound 101”).

In some aspects, the heterobifunctional compound has Formula IV and X is

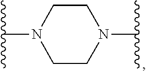 [SMILES: *N1CCN(*)CC1]

Y is C₂ alkylenyl, and Z is O (“Compound 102”).

In some aspects, the heterobifunctional compound has Formula III and X is

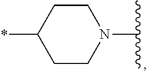 [SMILES: *C1CCN(*)CC1]

Y is C1 alkylenyl, and Z is

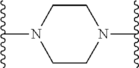 [SMILES: *N1CCN(*)CC1]

### (“Compound 103”).

In some aspects, the heterobifunctional compound has Formula V and X is O, R₁ and R₂ are each fluoride, and R₃ and R₄ are each hydrogen (“Compound 104”).

In some aspects, the heterobifunctional compound has Formula V and X is O, R₁ and R₄ are each fluoride and R₂ and R₃ are each hydrogen (“Compound 105”).

In some aspects, the heterobifunctional compound has Formula V and X is O, R₁ and R₃ are each fluoride, and R₂ and R₄ are each hydrogen (“Compound 106”).

In some aspects, the heterobifunctional compound has Formula V and X is O, R₃ and R₄ are each fluoride, and R₁ and R₂ are each hydrogen (“Compound 107”).

In some aspects, the degrader “D” binds a ubiquitin ligase.

In some aspects, the moiety that binds a ubiquitin ligase enzyme includes, but is not limited to, thalidomide, pomalidomide, lenalidomide, CC-885, CC-122, or CC-220.

In some aspects, the ubiquitin ligase is selected from the group consisting of ubiquitin-protein ligase E3A, mouse double minute 2 homolog (MDM2), anaphase promoting complex, ubiquitin-protein ligase E3 component n-recognin 5, suppressor of cytokine signaling/Cullin 5/RING, ligand of numb-protein X1 (LNX1), HAKAI/Casitas B-lineage lymphoma-transforming sequence-like protein 1 (CBLL 1); HECT domain and ankyrin repeat containing E3 ubiquitin protein ligase 1, HECT domain E3 ubiquitin protein ligase 4; HECT, C2, and WW domain containing E3 ubiquitin protein ligase 1 (HECW1); HECW2; HECT and RLD domain containing E3 ubiquitin protein ligase family member 1 (HERC1), HERC2, HERC3, HERC4, HERC5, HERC6; HECT, UBA, and WWE domain containing 1, E3 ubiquitin protein ligase; itchy E3 ubiquitin protein ligase; neural precursor cell expressed developmentally down-regulated protein 4 (NEDD4); NEDD4-like, peptidyl-prolyl cis-trans isomerase-like 2 (PPIL2); pre-mRNA-processing factor 19 (PRPF19), E3 SUMO-protein ligase protein inhibitor of activated STAT1 (PIAS1), PIAS2, PIAS3, PIAS4, RAN binding protein 2 (RANBP2), Ring finger protein 4, RING-box protein 1, SMAD specific E3 ubiquitin protein ligase 1 (SMURF1), SMURF2; STIP1 homology and U-box containing protein 1 (STUB1); TOP1 binding arginine/serine rich protein (TOPORS), thyroid hormone receptor interactor 12 (TRIP12), Ubiquitin-protein ligase E3A (UBE3A), UBE3B, UBE3C, UBE3D, UBE4A, UBE4B, U-box domain containing 5 (UBOX5), von Hippel-Lindau tumor suppressor, WW domain containing E3 ubiquitin protein ligase 1 (WWP1), WWP2, and PARKIN ligase. In some aspects, the ubiquitin ligase is E3 ubiquitin ligase.

In some aspects, the linker (L) of the compound of the disclosure can be a fluoro-phenyl substituted with one or more groups selected from the group consisting of a piperazine group, a piperidine group and a methoxyethoxy group. In some aspects, the linker is one of

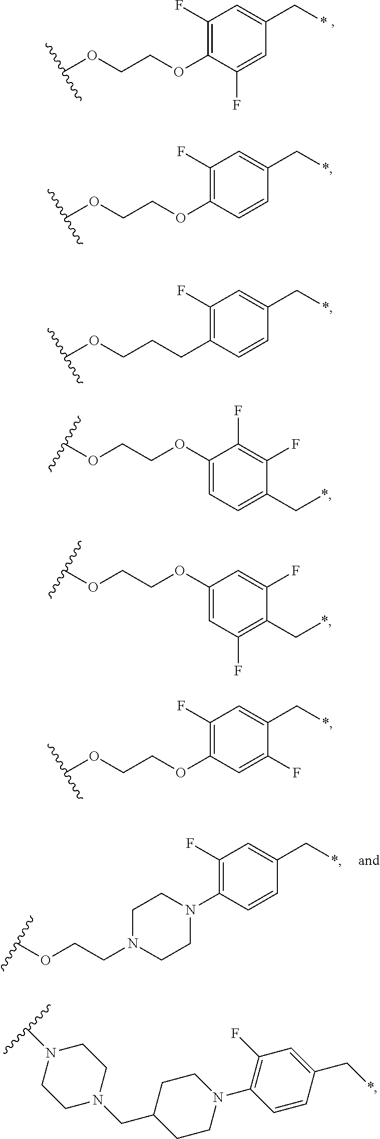

wherein the bond marked with an “*” is attached to the SMARCA2 binding moiety.

In some aspects, and without wishing to be bound by any one theory, the linker moiety provides target protein degradation because the linker provides optimal proximity and orientation of the degrader moiety and the target protein binding moiety such that the target protein bound by the target protein binding moiety is in optimal proximity and orientation with respect to the ubiquitin ligase bound by the ubiquitin ligase binding moiety so as to effectuate ubiquitination of the target protein by the ubiquitin ligase and, therefore, target protein degradation in the ubiquitin-proteasome system of a cell.

In some aspects, the target protein is SMARCA2 and one ligand is a SMARCA2 binding moiety.

In some aspects, the target protein is any protein that can be bound by the target binding moiety.

In some aspects, upon heterobifunctional compound-mediated heterodimerization of the ubiquitin ligase and the target protein, the target protein is ubiquitinated and degraded by the proteasome of the cell.

In some aspects, a heterobifunctional compound comprises a linker selected from

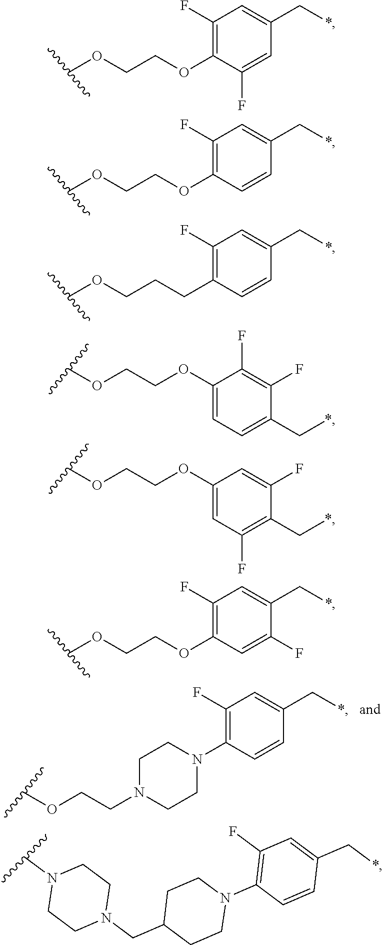

in combination with any target protein binding moiety and a ubiquitin ligase binding moiety, and wherein the bond marked with an “*” is attached to the target protein binding moiety.

In some aspects, and without wishing to be bound by any one theory, these linkers provide optimal proximity and orientation of the degrader moiety and a target protein binding moiety to enable target protein ubiquitination and degradation.

In some aspects, a compound of the disclosure has a DC₅₀ (concentration at which 50% of the target protein is degraded) for SMARCA2 of about 0.1 nM to about 300 nM, about 1 nM to about 200 nM, about 2 nM to about 150 nM, about 5 nM to about 120 nM, about 10 nM to about 100 nM, about 15 nM to about 80 nM, about 20 nM to about 70 nM, or about 30 nM to about 60 nM. In some aspects, a compound of the disclosure has a DC₅₀ for SMARCA2 of less than about 300 nM, less than about 200 nM, less than about 150 nM, less than about 120 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, or less than about 2 nM.

In some aspects, a compound of the disclosure degrades a SMARCA2 with a maximum degradation observed as percentage of DMSO control (Dₘₐₓ) of about 60% to about 100%, about 65% to about 99%, about 70% to about 95%, about 75% to about 90% or about 60%, 65%, 67%, 70%, 72%, 75%, 77%, 80%, 82%, 85%, 87%, 90%, 92%, 95%, 97%, 98%, 99%, or 100%, or a range between any preceding value. In some aspects, a compound of the disclosure degrades a SMARCA2 with a Dₘₐₓ of more than about 60%, more than about 65%, more than about 70%, more than about 75%, more than about 80%, more than about 85%, more than about 90%, more than about 95% or more than about 99%.

In some aspects, a compound of the disclosure has a DC₅₀ for SMARCA4 of about 500 nM to about 100 μM, about 700 nM to about 90 μM, about 800 nM to about 80 μM, about 900 nM to about 70 μM, about 1 M to about 60 μM, about 2 M to about 50 μM, about 5 M to about 25 μM, or about 10 M to about 20 μM. In some aspects, a compound of the disclosure has a DC₅₀ for SMARCA4 of more than about 500 nM, more than about 700 nM, more than about 1 μM, more than about 2 μM, more than about 5 μM, more than about 7 μM, or more than about 10 μM.

In some aspects, a compound of the disclosure degrades a SMARCA4 with a maximum degradation observed as percentage of DMSO control (Dₘₐₓ) of about 10% to about 80%, about 15% to about 70%, about 20% to about 60%, or about 25% to about 50%. In some aspects, a compound of the disclosure degrades a SMARCA4 with a Dₘₐₓ of less than about 80%, less than about 70%, less than about 60%, less than about 50%, less than about 40%, less than about 25%, or less than about 20%.

In some aspects, a compound of the disclosure has an IC₅₀ (concentration at which 50% of cellular proliferation is inhibited) of about 10 nM to about 50 μM, about 50 nM to about 20 μM, about 100 nM to about 10 μM, about 200 nM to about 8 μM, about 300 nM to about 5 μM, about 400 nM to about 2 μM, or about 500 nM to about 1 μM.

In some aspects, a compound of the disclosure has an IC₅₀ in wild-type SMARCA4 cells of about 900 nM to about 50 μM, about 1 M to about 40 μM, about 2 M to about M, about 5 M to about 25 μM, or about 10 M to about 20 μM. In some aspects, a compound of the disclosure has an IC₅₀ in wild-type SMARCA4 cells of more than about 900 nM, more than about 1 μM, more than about 2 μM, more than about 3 μM, more than about 5 μM, more than about 7 μM, or more than about 10 μM.

In some aspects, a compound of the disclosure has an IC₅₀ in mutant SMARCA4 cells of about 50 nM to about 10 μM, about 75 nM to about 5 μM, about 100 nM to about 1 μM, about 125 nM to about 900 nM, about 150 nM to about 800 nM, about 175 nM to about 700 nM, about 200 nM to about 600 nM, about 250 nM to about 500 nM. In some aspects, a compound of the disclosure has an IC₅₀ in mutant SMARCA4 cells of less than about 10 μM, less than about 5 μM, less than about 2 μM, less than about 1 μM, less than about 900 nM, less than about 800 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, or less than about 2 nM, or less than about 1 nM.

In some aspects, a compound of the disclosure competitively inhibits the binding of pomalidomide to cereblon. In some aspects, the compound of the disclosure competitively inhibits the binding of a SMARCA2 inhibitor to SMARCA2. In some aspects, a compound of the disclosure inhibits the proliferation of SMARCA4 mutant cells but does not inhibit the proliferation of SMARCA4 wild-type cells.

### III. Methods of Synthesis

In some aspects, the disclosure is directed to a method for making a compound of the disclosure, or a pharmaceutically acceptable salt thereof.

In some aspects, compounds of the disclosure can be synthesized according to the methods described herein in the Examples or below.

In some aspects, compounds of Formula (II) can be synthesized as shown in Schemes 1-4. Compound 1 is mixed with compound a in the presence of potassium carbonate and dimethylformamide to form compound 2 (Scheme 1).

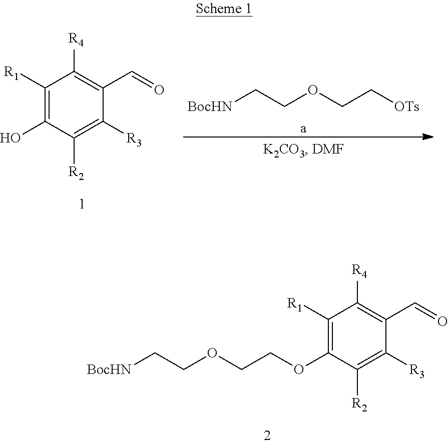

Compound 2 can be further reacted with compound b in the presence of sodium triacetoxyborohydride, N,N-Diisopropylethylamine, and Dimethylsulfoxide to form compound 3 (Scheme 2).

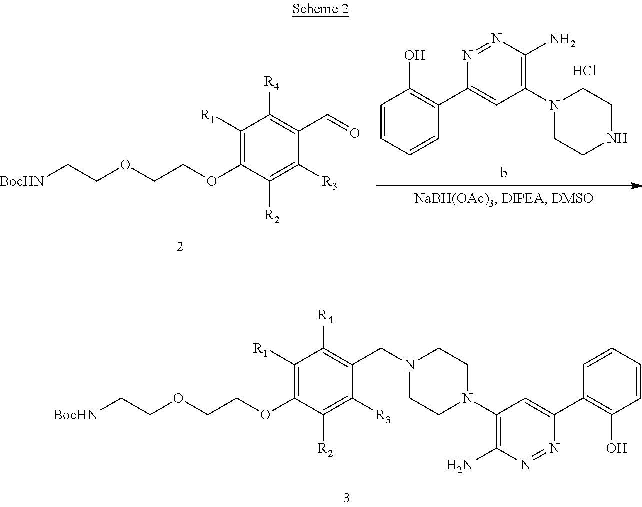

Compound 3 can be incubated with a mixture of hydrochloric acid and methanol and methanol to obtain compound 4 (Scheme 3).

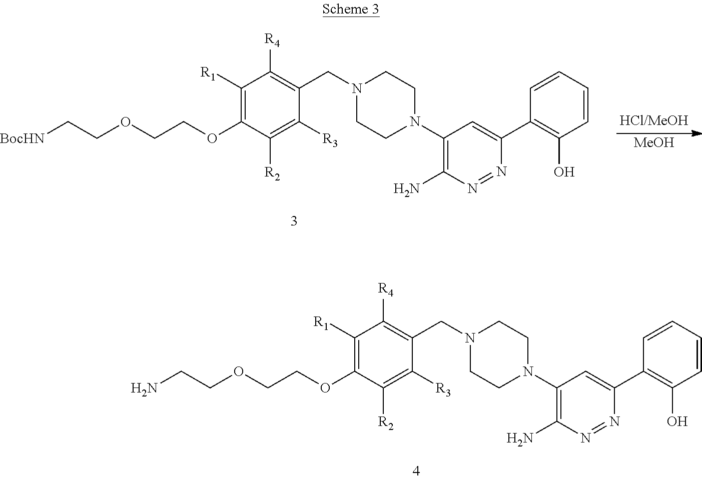

Compound 4 can be mixed with compound c in the presence of N,N-Diisopropylehtylamine and Dimethylformamide to form a heterobifunctional compound of Compound 101 and 104-108 as shown in Scheme 4.

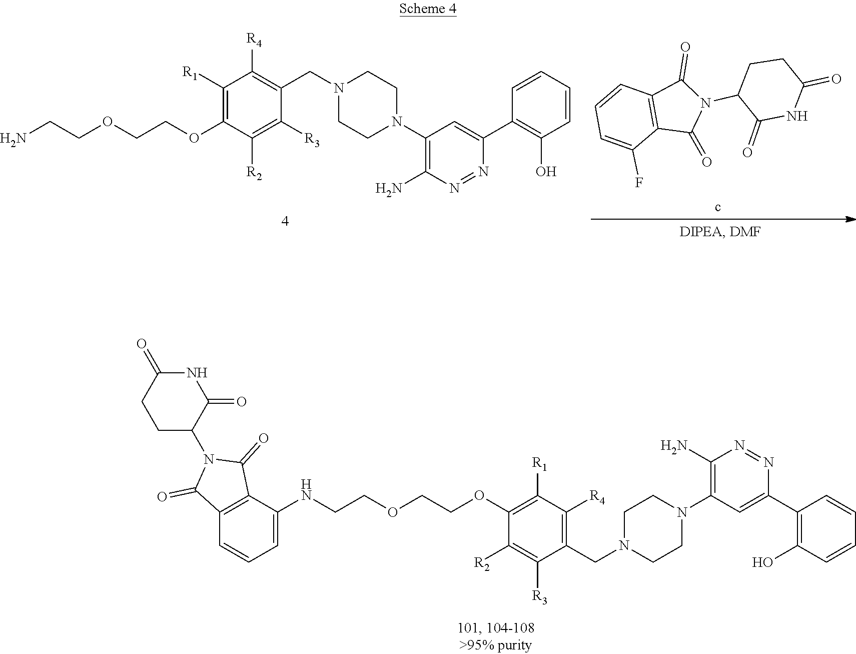

In some aspects, the compounds of the disclosure are produced at a purity of about 90%, about 91%, about 92%, about 93%, about 94%, about 95%, about 96%, about 97%, about 98%, about 99%, about 99.5%, or about 99.9%. In some aspects, the compounds of the disclosure are produced at a purity of at least about 90%, at least about 95%, at least about 96%, at least about 97%, at least about 98%, or at least about 99%.

### IV. Pharmaceutical Compositions

In some aspects, the disclosure is directed to a pharmaceutical composition that includes a therapeutically effective amount of a compound of the disclosure or a pharmaceutically acceptable salt or solvate thereof, and a pharmaceutically acceptable carrier, adjuvant or vehicle. The heterobifunctional compounds of the disclosure may be formulated into several different types of pharmaceutical compositions that contain a therapeutically effective amount of the heterobifunctional compound, and a pharmaceutically acceptable carrier, adjuvant, or vehicle.

In some aspects, the compounds of the disclosure or a pharmaceutically acceptable salt or solvate thereof, and a pharmaceutically acceptable carrier, adjuvant or vehicle are formulated for oral administration.

In some aspects, the compounds of the disclosure or a pharmaceutically acceptable salt or solvate thereof, and a pharmaceutically acceptable carrier, adjuvant or vehicle are formulated for parenteral administration. In some aspects, the pharmaceutical compositions for parenteral administration are for intravenous or subcutaneous administration. In some aspects, the compounds of the disclosure or a pharmaceutically acceptable salt or solvate thereof formulated for parenteral administration comprise a surfactant. In some aspects, the surfactant is a non-ionic surfactant including, but not limited to, solutol, polysorbate, Cremophor® EL, ELP, or RH40, a polyoxyethylene stearate, a sorbitan fatty acid ester, a polyoxyethylene alkyl ether, or a polyoxyehtylene nonylphenol ether.

In some aspects, the compounds of the disclosure or a pharmaceutically acceptable salt or solvate thereof are formulated for oral administration. In some aspects, the compounds of the disclosure or a pharmaceutically acceptable salt or solvate thereof are formulated as solids for oral administrations. In some aspects, the compounds of the disclosure or a pharmaceutically acceptable salt or solvate thereof are formulated as liquids for oral administration.

In some aspects, the pharmaceutical composition comprises the compounds of the disclosure or a pharmaceutically acceptable salt or solvate thereof, and a surfactant. In some aspects, the composition comprises a compound of the disclosure or a pharmaceutically acceptable salt or solvate thereof and a non-ionic surfactant. In some aspects, the non-ionic surfactant includes, but is not limited to, solutol, polysorbate, Cremophor® EL, ELP, or RH40, a polyoxyethylene stearate, a sorbitan fatty acid ester, a polyoxyethylene alkyl ether, or a polyoxyehtylene nonylphenol ether. In some aspects, the non-ionic surfactant is solutol, e.g., Kollophor® EL.

In some aspects, the pharmaceutical composition comprises the compounds of the disclosure or a pharmaceutically acceptable salt or solvate thereof and at least one metabolic stabilizer. In some aspects, the pharmaceutical composition comprises the compounds of the disclosure or a pharmaceutically acceptable salt or solvate thereof and an agent with CYP3A4 inhibitory activity. In some aspects, the agent with CYP3A4 inhibitory activity is ritonavir, atazanavir, darunavir, indinavir, lopinavir, nelfinavir, saquinavir, ceritinib, idelalisib, lonafarnib, tucatinib, clarithromycin, erythromycin, telithromycin, cobicistat, diltiazem, mifepristone, nefazodone, itraconazole, ketoconazole, levoketoconazole, posaconazole, or voriconazole. In some aspects, the pharmaceutical composition comprises a compound of the disclosure or a pharmaceutically acceptable salt or solvate thereof and ritonavir.

In some aspects, the pharmaceutical composition comprises the compounds of the disclosure or a pharmaceutically acceptable salt or solvate thereof and at least one metabolic stabilizer, wherein the compound is Compound 101, Compound 104, Compound 105, Compound 106, Compound 107, or Compound 108.

In some aspects, the pharmaceutical composition comprises a compound of the disclosure or a pharmaceutically acceptable salt or solvate thereof and does not comprise a metabolic stabilizer. In some aspects, the pharmaceutical composition comprises Compound 102 or Compound 103 or a pharmaceutically acceptable salt or solvate thereof and does not comprise a metabolic stabilizer.

In some aspects, the compounds of the disclosure or a pharmaceutically acceptable salt or solvate thereof, and a pharmaceutically acceptable carrier, adjuvant or vehicle are formulated for administration with at least one additional pharmaceutically active agent.

In some aspects, the compounds of the disclosure or a pharmaceutically acceptable salt or solvate thereof, and a pharmaceutically acceptable carrier, adjuvant or vehicle are formulated for simultaneous administration. In some aspects, the compound of disclosure or a pharmaceutically acceptable salt or solvate thereof, and a pharmaceutically acceptable carrier, adjuvant or vehicle are formulated for sequential administration.

In some aspects, a pharmaceutical composition comprising the compound of the disclosure or a pharmaceutically acceptable salt or solvate thereof is formulated for oral administration and a second pharmaceutical composition comprising at least one additional agent is formulated for parenteral administration. In some aspects, a pharmaceutical composition comprising the compound of Formula I, Formula II, Formula III, Formula IV, or Formula V or a pharmaceutically acceptable salt thereof is formulated for parenteral administration and a second pharmaceutical composition comprising at least one additional agent is formulated for oral administration

The compositions intended for oral use may contain one or more agents selected from the group consisting of inert, non-toxic pharmaceutically acceptable excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In some aspects, the compounds and/or additional therapeutic agent(s) described herein are formulated by combining the components with pharmaceutically acceptable carriers, adjuvants, excipients or vehicles formulated in oral dosage forms that include, by way of example only, tablets, powders, pills, dragees, capsules, liquids, gels, syrups, elixirs, slurries, suspensions, and the like.

In some aspects, pharmaceutical compositions for oral use are obtained by mixing one or more solid excipient with the compounds and/or additional therapeutic agent(s) described herein, optionally grinding the resulting mixture, and processing the mixture of granules, after adding suitable auxiliaries, if desired, to obtain tablets or dragee cores.

Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate.

In some aspects, dosage forms, such as dragee cores and tablets, are provided with one or more suitable coating. In some aspects, concentrated sugar solutions are used for coating the dosage form. The sugar solutions optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In some aspects, therapeutically effective amounts of the compounds of the disclosure or a pharmaceutically acceptable salt or solvate thereof and/or additional therapeutic agent(s) described herein are formulated into other oral dosage forms. A unit dosage form comprises physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose. Oral dosage forms include push-fit capsules made of gelatin, as well as soft, sealed capsules made of gelatin and a plasticizer, such as glycerol or sorbitol. In some aspects, push-fit capsules contain the active ingredients in admixture with one or more filler. Fillers include, by way of example only, lactose, binders such as starches, and/or lubricants such as talc or magnesium stearate and, optionally, stabilizers. In some aspects, soft capsules contain one or more active compound that is dissolved or suspended in a suitable liquid. Suitable liquids include, by way of example only, one or more fatty oil, liquid paraffin, or liquid polyethylene glycol. In addition, stabilizers are optionally added. In some aspects, a stabilizer is a CYP3A4 inhibitor. In some aspects, the pharmaceutical composition is a liquid dosage form suitable for oral administration. In some aspects, a liquid formulation comprises at least one of a buffer, a tonicity agent, an inorganic salt, a surfactant, a metabolic stabilizer, or a combination thereof.

In some aspects, the pharmaceutical composition is a liquid dosage form suitable for parenteral administration. In some aspects, the pharmaceutical composition is suitable for intravenous administration. In some aspects, the pharmaceutical composition is suitable for intravenous infusion. In some aspects, the pharmaceutical composition is suitable for injection. In some aspects, the pharmaceutical composition is suitable for subcutaneous injection. In some aspects, the compositions for parenteral administration optionally further comprise one or more additional therapeutic agent(s). In some aspects, the pharmaceutical composition for intravenous administration comprises at least one of a buffer, a tonicity agent, an inorganic salt, a surfactant, a metabolic stabilizer, or a combination thereof.

In some aspects, the buffer comprises a phosphate bicarbonate buffered solution.

In some aspects, the inorganic salt comprises sodium chloride, calcium chloride, potassium chloride, magnesium chloride, sodium bicarbonate, mono-potassium phosphate, or sodium phosphate dibasic.

In some aspects, the tonicity agent is dextrose, glycerin, mannitol, potassium chloride, or sodium chloride.

In some aspects, the surfact is an anionic surfact, a cationic surfactant, an amphoteric surfact, or a non-ionic surfactant. In some aspects, an anionic surfactant includes, but is not limited to, sodium laurylsulfate and docusate sodium. In some aspects, a cationic surfactant includes, but is not limited to, benzalkonium chloride, cetylpyridinium chloride, or a phosphatide. In some aspects, an amphoteric surfactant includes, but is not limited to, betaine, sulfobetaine, an amino acid, or a phospholipid. In some aspects, a non-ionic surfactant includes, but is not limited to, polysorbate, solutol, Cremophor® EL, ELP, or RH40, a polyoxyethylene stearate, a sorbitan fatty acid ester, a polyoxyethylene alkyl ether, or a polyoxyehtylene nonylphenol ether.

In some aspects, the pharmaceutical composition may be administered orally, parenterally, by inhalation spray, topically, rectally, nasally, buccally, vaginally or via an implanted reservoir.

In some aspects, the pharmaceutical composition comprising the compound of Formula I, Formula II, Formula III, Formula IV, or Formula V or a pharmaceutically acceptable salt thereof and the at least one additional pharmaceutically active agent can be administered by the same route or by different routes. For example, a first therapeutic agent of the combination may be administered by intravenous injection while the other therapeutic agent or agents of the combination may be administered orally. Alternatively, for example, all therapeutic agents may be administered orally or all therapeutic agents may be administered by intravenous injection.

### V. Medicament

In some aspects, the present disclosure relates to use of a compound of the disclosure or a pharmaceutically acceptable salt or solvate thereof in the manufacture of a medicament for the treatment of a tumor in a patient in need of such treatment.

In some aspects, the medicament comprises a compound of the disclosure or a pharmaceutically acceptable salt or solvate thereof, and can optionally comprise one or more additional therapeutic agents. In some aspects, the medicament is in single dosage form or in separate dosage forms.

In some aspects, the present disclosure relates to the use of a compound of the disclosure or a pharmaceutically acceptable salt or solvate thereof in the manufacture of a medicament for the treatment of a tumor. In some aspects, the medicament comprising the compound of the disclosure or a pharmaceutically acceptable salt or solvate thereof and at least one additional therapeutic are administered simultaneously. In some aspects, the medicament comprising the compound of the disclosure or a pharmaceutically acceptable salt or solvate thereof and the medicament comprising the at least one additional therapeutic are administered separately. In some aspects, the medicament comprising the compound of the disclosure or a pharmaceutically acceptable salt or solvate thereof and the medicament comprising the at least one additional therapeutic are administered sequentially.

### VII. Methods of Treatment

In some aspects, the disclosure provides methods of using an effective amount of the compounds of the disclosure or a pharmaceutically acceptable salt or solvate thereof for the treatment or amelioration of a disease or condition, such as cancer, e.g., a SMARCA4-deficient cancer, including lung cancer, liver cancer, bladder cancer, breast cancer or colon cancer.

In some aspects, provided are methods of treating a condition or disease by administering a therapeutically effective amount of a compound of the disclosure or a pharmaceutically acceptable salt or solvate thereof to a subject in need thereof. In some aspects, the method of treatment comprises administering a therapeutically effective amount of a composition comprising a compound of the disclosure or a pharmaceutically acceptable salt or solvate thereof and at least one additional pharmaceutically active agent. In some aspects, the method of treatment comprises administering a therapeutically effective amount of a composition comprising a compound of the disclosure or a pharmaceutically acceptable salt or solvate thereof, an excipient, and at least one additional pharmaceutically active agent. In some aspects, the method of treatment comprises administering a therapeutically effective amount of a composition comprising a compound of the disclosure or a pharmaceutically acceptable salt or solvate thereof, an excipient, and a metabolic stabilizer. In some aspects, the method of treatment comprises administering a therapeutically effective amount of a composition comprising a compound of the disclosure or a pharmaceutically acceptable salt or solvate thereof, an excipient, and an agent with CYP3A4 inhibitory activity. In some aspects, the agent with CYP3A4 inhibitory activity is ritonavir.

In some aspects, the method of treatment comprises administering a therapeutically effective amount of a composition comprising a compound of the disclosure or a pharmaceutically acceptable salt or solvate thereof and a surfactant. In some aspects, the surfactant is an anionic surfact, a cationic surfactant, an amphoteric surfact, or a non-ionic surfactant. In some aspects, the method of treatment comprises administering a therapeutically effective amount of a composition comprising a compound of the disclosure or a pharmaceutically acceptable salt or solvate thereof and an anionic surfactant, a cationic surfactant, an amphoteric surfactant, or a non-ionic surfactant. In some aspects, an anionic surfactant includes, but is not limited to, sodium laurylsulfate and docusate sodium. In some aspects, a cationic surfactant includes, but is not limited to, benzalkonium chloride, cetylpyridinium chloride, or a phosphatide. In some aspects, an amphoteric surfactant includes, but is not limited to, betaine, sulfobetaine, an amino acid, or a phospholipid. In some aspects, a non-ionic surfactant includes, but is not limited to, polysorbate, solutol, Cremophor® EL, ELP, or RH40, a polyoxyethylene stearate, a sorbitan fatty acid ester, a polyoxyethylene alkyl ether, or a polyoxyehtylene nonylphenol ether.

In some aspects, the method of treatment comprises administering a therapeutically effective amount of a composition comprising a compound of the disclosure or a pharmaceutically acceptable salt or solvate thereof and a non-ionic surfactant. In some aspects, the method of treatment comprises administering a therapeutically effective amount of a composition comprising a compound of the disclosure or a pharmaceutically acceptable salt or solvate thereof and solutol. In some aspects, the method of treatment comprises administering a therapeutically effective amount of a composition comprising a compound of the disclosure or a pharmaceutically acceptable salt or solvate thereof, solutol, and ritonavir.

In some aspects, the disease or condition is treatable by degradation of SMARCA2 in the cancer. In some aspects, the disease treated with a compound of the disclosure or a pharmaceutically acceptable salt or solvate thereof is a chronic autoimmune disorder, an inflammatory condition, a proliferative disorder, a sepsis, or a viral infection. In some aspects, the compounds of the disclosure or a pharmaceutically acceptable salt or solvate thereof reduces proliferation of unwanted cells by inducing apoptosis in the cells.

In some aspects, the compositions comprising a compound of any of Formula I, Formula II, Formula III, Formula IV, or Formula V or a pharmaceutically acceptable salt thereof and a pharmaceutically acceptable carrier, adjuvant or vehicle for treating a disease or condition that is dependent upon altered activity of a SWI/SNF complex.

In some aspects, the disease/condition/cancer to be treated with the compound of Formula I, Formula II, Formula III, Formula IV, or Formula V or a pharmaceutically acceptable salt thereof is a disease/condition/cancer defined as exhibiting one or more of the following: impairment or loss of function of BAF complex subunits, including but not limited to SMARCB1, ARID1A, ARID1B, ARID2, PBRM1, and SMARCA4 due either to inactivating mutations in these genes or loss of their expression through alternative mechanisms other than inactivating mutations; impairment or loss of SMARCA4 function due either to inactivating mutations in the SMARCA4 gene or loss of SMARCA4 expression through alternative mechanisms other than inactivating mutations. In some aspects, the disease/condition/cancer is defined as exhibiting a SMARCA4 deletion. In some aspects, the SMARCA4 deletion is a heterozygous or homozygous SMARCA4 gene truncation.

In some aspects, inactivating mutations affecting SMARCA4 function include heterozygous or homozygous nonsense or insertion/deletion (e.g. frameshift) mutations that result in loss of protein or activity; and/or heterozygous or homozygous missense mutations that inactivate the function of the protein; and/or changes in gene expression levels; and/or changes in protein levels; and/or changes in protein function.

In some aspects, the cancer has a mutation in SMARCA4 and/or shows loss of SMARCA4 function while at the same time retaining a functional copy of a SMARCA2 gene and/or expression of a functional SMARCA2 protein.

In some aspects, the diseases or conditions can be cancers of the head and neck, e.g., cancers of the nasal cavity, paranasal sinuses, nasopharynx, oral cavity, oropharynx, middle ear, larynx, hypopharynx, salivary glands; cancers of the lung, e.g., non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer); neoplasms of the mediastinum, e.g., neurogenic tumors (including neurofibroma, neurilemoma, malignant schwannoma, neurosarcoma, ganglioneuroblastoma, anglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma); cancers of the gastrointestinal (Gl) tract, e.g., cancers of the esophagus, stomach (gastric cancer), pancreas, liver, biliary tree, gall bladder, small intestine (including duodenum, jejunum, ileum), large intestine (including cecum, colon, rectum, anus), colorectal cancer, gastrointestinal stroma tumor, hepatocellular carcinoma (HCC), hepatoblastoma, cholangiocarcinoma, cholangiocellular carcinoma, hepatic cystadenocarcinoma, angiosarcoma, hemangioendothelioma, leiomyosarcoma, malignant Schwannoma, fibrosarcoma; cancer of the genitourinary system (including kidney, e.g. renal pelvis, renal cell carcinoma (RCC), nephroblastoma (Wilms' tumor), hypernephroma, cancer of the ureter; urinary bladder, urethra, penis, testis; gynecologic cancer, e.g., cancer of the ovary, fallopian tube, peritoneum, cervix, vulva, vagina, uterine body; cancers of the breast, e.g., mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast; cancers of the endocrine system, e.g., cancers of the endocrine glands, thyroid gland (thyroid carcinomas/tumors; papillary, follicular, anaplastic, medullary), parathyroid gland (parathyroid carcinoma), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, gastrinoma, insulinoma, glucagonoma, somatostatinoma, carcinoid tumors; sarcomas of the soft tissues, e.g., fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma epithelioid sarcoma, extrarenal rhabdoid tumor, desmoplastic small cell tumor; sarcomas of the bone, e.g., myeloma, reticulum cell sarcoma, chondrosarcoma (including central, peripheral, clear cell, mesenchymal chondrosarcoma), osteosarcoma, Ewing's tumor, malignant giant cell tumor, histiocytoma, fibrosarcoma, chordoma, hemangioendothelioma, hemangiopericytoma, osteochondroma, osteoid osteoma, osteoblastoma, eosinophilic granuloma, chondroblastoma; mesothelioma: e.g. pleural mesothelioma, peritoneal mesothelioma; cancers of the skin, e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain, e.g., astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors; lymphomas and leukemias, e.g., B-cell non-Hodgkin lymphomas (NHL) including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML); or a cancer of unknown primary site (CUP). The cancers enumerated are meant to include the primary tumors and any metastatic tumors derived therefrom.

In some aspects, the cancer treated by a compound of the disclosure or a pharmaceutically acceptable salt or solvate thereof is a lung cancer, a liver cancer, a bladder cancer, a breast cancer or a colon cancer. In some aspects, the cancer is a non-small cell lung cancer.

### VII. Combination Therapy

In some aspects, a compound of Formula I, Formula II, Formula III, Formula IV, or Formula V, or a pharmaceutically acceptable salt thereof, is administered to a subject before, after or together with at least one other pharmaceutically active agent.

In some aspects, the pharmacologically active agent to be used in combination with the compound of Formula I, Formula II, Formula III, Formula IV, or Formula V, or a pharmaceutically acceptable salt thereof, can be selected from any one or more of the following: hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide); aromatase inhibitors (e.g. anastrozole, letrozole, liarozole, vorozole, exemestane, atamestane); LHRH agonists and antagonists (e.g. goserelin acetate, luprolide); inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumour antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod); tubuline inhibitors; DNA synthesis inhibitors; PARP inhibitors; topoisomerase inhibitors (e.g. epipodophyllotoxins such as for example etoposide and etopophos, teniposide, amsacrin, topotecan, irinotecan, mitoxantrone); serine/threonine kinase inhibitors (e.g. PDK 1 inhibitors, Raf inhibitors, A-Raf inhibitors, B-Raf inhibitors, CRaf inhibitors, mTOR inhibitors, mTORC1/2 inhibitors, PI3K inhibitors, PI3Ka inhibitors, dual mTOR/PI3K inhibitors, STK 33 inhibitors, AKT inhibitors, PLK 1 inhibitors, inhibitors of CDKs, Aurora kinase inhibitors); tyrosine kinase inhibitors (e.g. cetuximab, gefitinib, afatinib, nintedanib, imatinib, lapatinib, bosutinib, PTK2/FAK inhibitors); protein protein interaction inhibitors (e.g. IAP activator, Mcl-1, MDM2/MDMX); MEK inhibitors; ERK inhibitors; FLT3 inhibitors; BRD4 inhibitors; IGF-1 R inhibitors; TRAILR2 agonists; Bcl-xL inhibitors; Bcl-2 inhibitors; ErbB receptor inhibitors; BCR-ABL inhibitors; ABL inhibitors; Src inhibitors; rapamycin analogs (e.g. everolimus, temsirolimus, ridaforolimus, sirolimus); androgen synthesis inhibitors; androgen receptor inhibitors; DNMT inhibitors; HDAC inhibitors; ANG1/2 inhibitors; CYP17 inhibitors; radiopharmaceuticals; immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19), PSMA×CD3), tumor vaccines; and various chemotherapeutic agents such as amifostin, anagrelid, clodronat, filgrastin, interferon, interferon alpha, leucovorin, procarbazine, levamisole, mesna, mitotane, pamidronate and porfimer.

In some aspects, when two or more active pharmaceutically agents are to be used as part of a combined treatment regimen, they can be administered via the same route of administration or via different routes of administration, at essentially the same time (i.e. simultaneously, concurrently) or at different times (e.g. sequentially, successively, alternately, consecutively, or according to any other sort of alternating regime).

In some aspects, when the active pharmaceutically agents are administered simultaneously via the same route of administration, they may be administered as different pharmaceutical compositions or as part of a combined pharmaceutical composition. When two or more active pharmaceutically agents are used as part of a combined treatment regimen, each of the active pharmaceutically agents may be administered in the same amount and according to the same regimen as used when the active pharmaceutically agent is used on its own.

In some aspects, when the compound of the disclosure and the at least one additional therapeutic are administered sequentially, they are administered to the patient at two different time points that are separated by 16 minutes or more, for example, 18 minutes, 20 minutes, 25 minutes, 30 minutes, 35 minutes, 40 minutes, 45 minutes, 50 minutes, 55 minutes, 60 minutes, 70 minutes, 80 minutes or 90 minutes, 2 hours, 5 hours, 8 hours, 12 hours, 16 hours, 24 hours; 2, 3, 4, 5, 6, 7, 8, 9, 10, 11, 12, 13, 14 days; or 3, 4, 5, 6, 7, 8, 9, 10, 11 or 12 weeks.

In some aspects, when the compound of the disclosure and the at least one additional therapeutic are administered concomitantly to the patient, they are administered at the same time or almost at the same time. For example, the compound of the disclosure, or a pharmaceutically acceptable salt thereof, and at least one additional therapeutic agent are administered “almost at the same time,” when they are administered no more than about 15, 14, 13, 12, 11, 10, 9, 8, 7, 6, 5, 4, 3, 2 minutes, or 1 minute apart.

In some aspects, the compound of the disclosure and the at least one additional therapeutic are administered with an intermission that is a rest period that is subsequent to the administration of the compound of the disclosure and the at least one additional therapeutic, which rest period can be at least one day.

### VIII. Kits

In some aspects, kits are provided that comprise a pharmaceutical composition or dosage form comprising a compound of the disclosure or a pharmaceutically acceptable salt or solvate thereof and one or more pharmaceutically acceptable carriers, excipients, and/or vehicles. In some aspects, kits are provided that comprise: a first pharmaceutical composition or dosage form comprising a compound of the disclosure or a pharmaceutically acceptable salt or solvate thereof and one or more pharmaceutically acceptable carriers, excipients, and/or vehicles, and, optionally, a second pharmaceutical composition or dosage form comprising another pharmaceutically active agent and one or more pharmaceutically acceptable carriers, excipients and/or vehicles.

In some aspects, the kit comprises a medicament or a combination of medicaments packaged in a manner that facilitates their use to practice methods of the present disclosure.

In some aspects, the kit further comprises instructions on how the pharmaceutical composition or compositions are to be administered and tools for the preparation of and administration of the pharmaceutical composition via, e.g., parenteral route.

### EXAMPLES

### Example 1—Preparation of Compound 101

### General Procedure for Preparation of Compound a

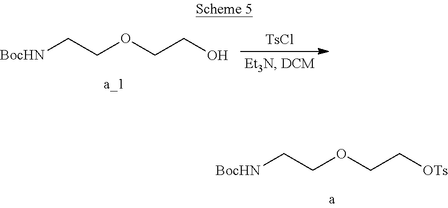 [SMILES: CC(C)(C)OC(=O)NCCOCCO ; "TsCl" ; Cc1ccc(S(=O)(=O)OCCOCCNC(=O)OC(C)(C)C)cc1]

To a solution of compound a_1 (100 g, 487 mmol, 1.00 eq) in DCM (750 mL) was added Et₃N (148 g, 1.46 mol, 203 mL, 3.00 eq) and TsCl (139 g, 731 mmol, 1.50 eq), the mixture was stirred at 25° C. for 1 hr (Scheme 5).

LCMS showed the reaction was consumed completely. The mixture was diluted with H₂O (500 mL), then extracted with DCM (500 mL×3). The combined organic layers were washed with brine (500 mL), dried over Na₂SO₄, filtered and concentrated under reduced pressure to give a residue.

The crude product was triturated with MTBE/n-heptane (3: 1, 80.0 mL) at 0° C. for 30 mins. The mixture was filtered, the cake was collected, dried in vacuum to afford compound a (171 g, 430 mmol, 88.3% yield, 90.4% purity) as a white solid.

LCMS: Rt=0.916 min, m/z: 260 (M+H−Boc)⁺.

HPLC: Rt=2.98 min.

¹H NMR: (400 MHz, CDCl₃)

δ 7.81 (d, J=8.4 Hz, 2H), 7.35 (d, J=8.0 Hz, 2H), 4.16˜4.14 (m, 2H), 3.63˜3.61 (m, 2H), 3.45˜3.43 (m, 2H), 3.24˜3.20 (m, 2H), 3.14˜3.10 (m, 1H), 2.44 (s, 3H), 1.44 (s, 9H).

### General Procedure for Preparation of Compound 6

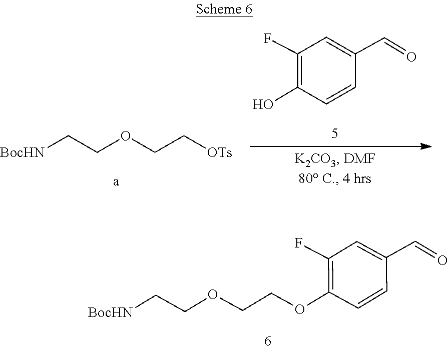 [SMILES: O=Cc1ccc(O)c(F)c1 ; Cc1ccc(S(=O)(=O)OCCOCCNC(=O)OC(C)(C)C)cc1 ; CC(C)(C)OC(=O)NCCOCCOc1ccc(C=O)cc1F]

To a solution of compound a (35.0 g, 250 mmol, 1.00 eq) and compound 5 (98.7 g, 275 mmol, 1.10 eq) in DMF (350 mL) was added K₂CO₃ (86.3 g, 624 mmol, 2.50 eq), the mixture was stirred at 90° C. for 2 hrs (Scheme 6). LCMS showed the reaction mixture was consumed completely. The mixture was diluted with H₂O (1.00 L), then extracted with EtOAc (500 mL×3). The combined organic layers washed with brine (500 mL×2), dried over Na₂SO₄, filtered and concentrated under reduced pressure to give a residue. The crude product was triturated with n-heptane for 30 mins to afford compound 6 (72.0 g, 219 mmol, 87.7% yield, 99.6% purity) as a light yellow solid.

LCMS: Rₜ=0.870 min, m/z=228.2 (M−56)

HPLC: Rₜ=2.73 min

¹H NMR: (400 MHz, CDCl₃)

δ 9.87˜9.86 (m, 1H), 7.64˜7.60 (m, 2H), 7.10 (t, J=8.0 Hz, 1H), 4.89 (s, 1H), 4.29˜4.27 (m, 2H), 3.89˜3.87 (m, 2H), 3.63˜3.61 (m, 2H), 3.35˜3.34 (m, 2H), 1.44 (s, 9H).

### General Procedure for Preparation of Compound 7

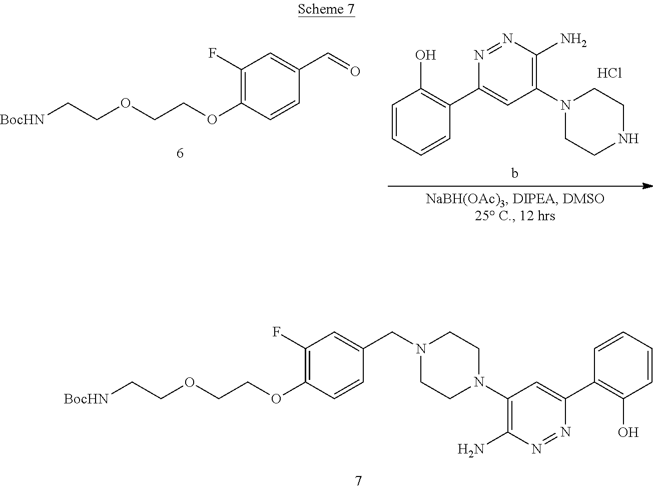 [SMILES: CC(C)(C)OC(=O)NCCOCCOc1ccc(C=O)cc1F ; Nc1nnc(-c2ccccc2O)cc1N1CCNCC1 ; Cl ; CC(C)(C)OC(=O)NCCOCCOc1ccc(CN2CCN(c3cc(-c4ccccc4O)nnc3N)CC2)cc1F]

To a solution of compound b (42.3 g, 137 mmol, 1.00 eq, HCl) in DMSO (450 mL) was added DIEA (17.7 g, 137 mmol, 23.9 mL, 1.00 eq) and stirred at 20° C. for 0.5 hr (Scheme 7). Then the mixture was added compound 6 (45.0 g, 137 mmol, 1.00 eq) and NaBH(OAc)₃ (58.3 g, 275 mmol, 2.00 eq). The mixture was stirred at 20° C. for 12 hrs. LCMS showed the reaction mixture was consumed completely. The mixture was diluted with H₂O (100 mL), then extracted with EtOAc (100 mL×3). The combined organic layers washed with brine (100 mL×2), dried over Na₂SO₄, filtered and concentrated under reduced pressure to give a residue. The residue was purified by column chromatography (SiO₂, Petroleum ether/Ethyl acetate=3/1 to 0/1, Rf=0.20) to afford compound 7 (57.0 g, 96.9 mmol, 70.5% yield, 99.1% purity) as a yellow solid.

LCMS: Rₜ=1.02 min, m/z=583.3 (M+1)

HPLC: Rₜ=3.68 min

¹H NMR: (400 MHz, DMSO-d₆)

δ 14.10 (s, 1H), 7.91 (dd, J=1.6 Hz, J=8.4 Hz, 1H), 7.51 (s, 1H), 7.25˜-7.21 (m, 1H), 7.18˜7.11 (m, 2H), 7.08˜7.06 (m, 1H), 6.90˜6.86 (m, 2H), 6.78 (t, J=5.6 Hz, 1H), 6.24 (s, 2H), 4.16˜4.13 (m, 2H), 3.74˜3.72 (m, 2H), 3.50 (s, 2H), 3.45 (t, J=6.4 Hz, 2H), 3.11˜3.07 (m, 5H), 2.58 (m, 4H), 1.36 (s, 9H).

### General Procedure for Preparation of Compound 8

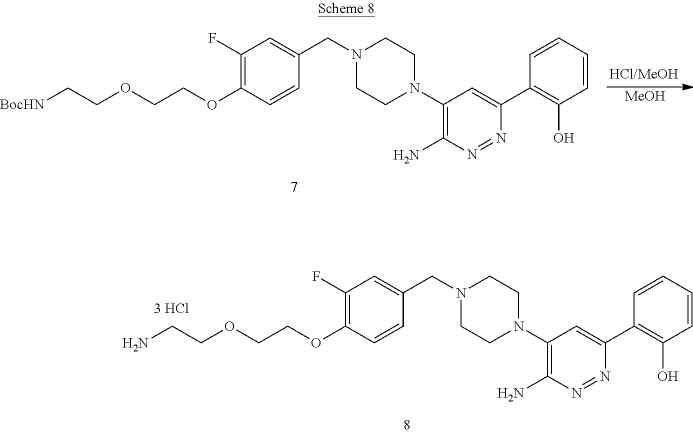 [SMILES: "MeOH" ; CC(C)(C)OC(=O)NCCOCCOc1ccc(CN2CCN(c3cc(-c4ccccc4O)nnc3N)CC2)cc1F ; NCCOCCOc1ccc(CN2CCN(c3cc(-c4ccccc4O)nnc3N)CC2)cc1F]

To a solution of compound 7 (57.0 g, 97.8 mmol, 1.00 eq) in MeOH (250 mL) was added HCl/MeOH (4 M, 244.6 mL, 10.0 eq), the mixture was stirred at 15° C. for 12 hrs (Scheme 8). LCMS showed the reaction mixture was consumed completely. The mixture was concentrated under reduced pressure to give a residue. The crude product was triturated with MTBE for 1 hr to afford compound 8 (52.0 g, 80.3 mmol, 81.8% yield, 91.1% purity, 3HCl) as a yellow solid.

LCMS: Rₜ=1.01 min, m/z=483.2 (M+1)

¹H NMR: (400 MHz, MeOD-d₄)

δ 7.71 (s, 1H), 7.65 (dd, J=1.2 Hz, J=8.0 Hz, 1H), 7.51 (dd, J=2.0 Hz, J=11.6 Hz, 1H), 7.45˜7.41 (m, 2H), 7.26 (t, J=8.8 Hz, 1H), 7.03˜7.06 (m, 2H), 4.45 (s, 2H), 4.31˜4.29 (m, 2H), 3.97˜3.93 (m, 4H), 3.82 (t, J=5.2 Hz, 2H), 3.64˜3.58 (m, 3H), 3.55˜3.52 (m, 1H), 3.48˜3.42 (m, 2H), 3.35 (s, 2H), 3.18˜3.15 (m, 2H).

### General Procedure for Preparation of Compound 101

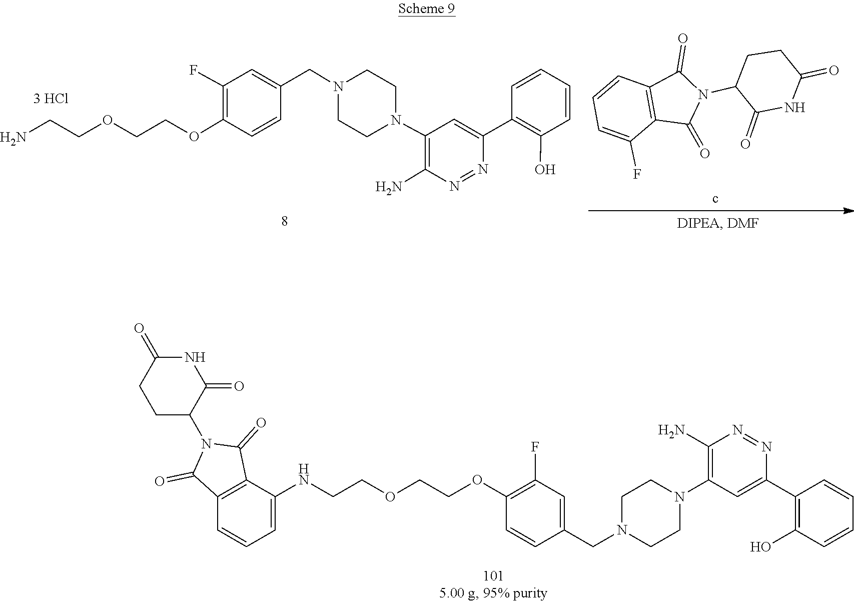

To a solution of compound 8 (30.0 g, 50.8 mmol, 1.00 eq, 3HCl) in DMF (300 mL) was added DIEA (39.4 g, 305 mmol, 6.00 eq) and compound c (14.0 g, 50.8 mmol, 1.00 eq) (Scheme 9). The mixture was stirred at 90° C. for 12 hrs. LCMS showed the reaction mixture was consumed completely. The mixture was diluted with H₂O (1.00 L), the extracted with EtOAc (500 mL×3). The combined organic layers washed with brine (500 mL×2), dried over Na₂SO₄, filtered and concentrated under reduced pressure to give a residue. The residue was purified by column chromatography (SiO₂, Petroleum ether/Ethyl acetate=3/1 to 0/1 and Dichloromethane/methyl alcohol=50/1, Rf=0.38). Then the crude product was triturated with ACN at 15° C. for 30 min to afford Compound 101 (8.6 g, 11.7 mmol, 23.0% yield, 96.1% purity) as a yellow solid. A total amount of 5 g of Compound 101 were produced at a purity of 95%.

LCMS: Rₜ=0.996 min, m/z=739.3 (M+1)

HPLC: Rₜ=3.29 min

¹H NMR: (400 MHz, CDCl₃)

δ 13.77 (s, 1H), 9.84 (s, 1H), 7.59˜7.57 (m, 1H), 7.47 (t, J=8.0 Hz, 1H) 7.28˜7.25 (m, 2H), 7.11˜6.99 (m, 5H), 6.91˜6.87 (m, 2H), 6.54 (t, J=5.6 Hz, 1H), 4.97˜4.88 (m, 3H), 4.25˜4.23 (m, 2H), 3.83 (t, J=5.2 Hz, 2H), 3.62˜3.59 (m, 1H), 3.49˜3.43 (m, 3H), 3.15 (s, 4H), 2.96˜2.68 (m, 7H).

### Example 2—Preparation of Compound 104

### General Procedure for Preparation of Compound 9

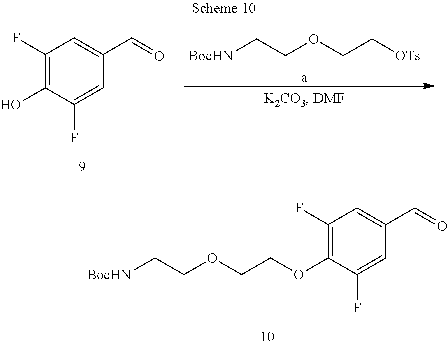

To a solution of compound a (2.50 g, 6.96 mmol, 1.10 eq) and compound 9 (1.00 g, 6.33 mmol, 1.00 eq) in DMF (25.0 mL) was added K₂CO₃ (2.19 g, 15.8 mmol, 2.50 eq), the mixture was stirred at 90° C. for 16 hrs (Scheme 10).

LCMS showed the reaction mixture was consumed completely. The mixture was diluted with H₂O (100 mL), then extracted with EtOAc (70.0 mL×3). The combined organic layers were washed with brine (70.0 mL×2), dried over Na₂SO₄, filtered and concentrated under reduced pressure to give compound 10 (2.20 g, 6.25 mmol, 98.8% yield, 98.1% purity) as a yellow oil and it was used for next step directly.

LCMS: Rₜ=0.898 min, m/z: 246 (M+H−Boc)⁺.

HPLC: Rₜ=2.88 min.

¹H NMR: (400 MHz, CDCl₃)

δ 9.85 (t, J=1.6 Hz, 1H), 7.46 (d, J=8.4 Hz, 2H), 4.86 (brs, 1H), 4.45˜4.43 (m, 2H), 3.81˜3.79 (m, 2H), 3.58˜3.56 (m, 2H), 3.31˜3.29 (m, 2H), 1.44 (s, 9H).

### General Procedure for Preparation of Compound 11

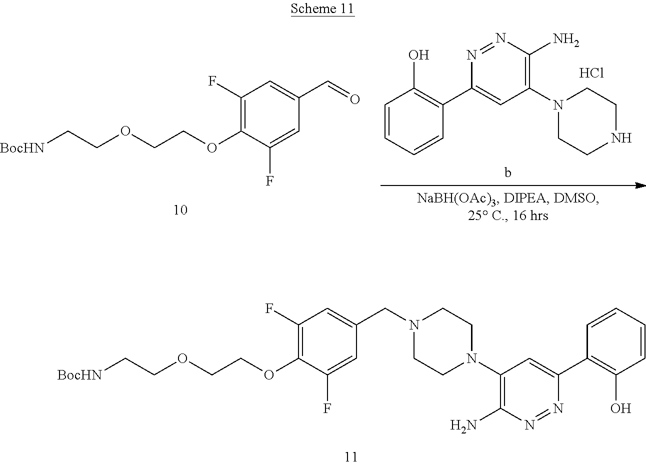

To a solution of compound b (891 mg, 2.90 mmol, 1.00 eq, HCl) in DMSO (15.0 mL) was added DIPEA (374 mg, 2.90 mmol, 504 μL, 1.00 eq), the mixture was stirred at 20° C. for 15 mins (Scheme 11). Then compound 10 (1.00 g, 2.90 mmol, 1.00 eq) and NaBH(OAc)₃ (1.23 g, 5.79 mmol, 2.00 eq) was added, the mixture was stirred at 20° C. for 12 hrs. LCMS showed the reaction mixture was consumed completely. The mixture was quenched with aq. NaHCO₃ (sat, 12.0 mL), then H₂O (60.0 mL) was added, the resulting mixture was extracted with EtOAc (40.0 mL×3). The combined organic layers were washed with brine (50.0 mL×2), and concentrated under reduced pressure to give a residue. The residue was purified by column chromatography (SiO₂, Petroleum ether/Ethyl acetate=5/1 to 1/2) to afford compound 11 (880 mg, 1.42 mmol, 49.1% yield, 97.0% purity) as a yellow solid.

LCMS: Rₜ=1.05 min, m/z: 601 (M+H)⁺.

HPLC: Rₜ=3.67 min.

¹H NMR: (400 MHz, CDCl₃)

δ 13.7 (s, 1H), 7.53 (d, J=7.6 Hz, 1H), 7.27˜7.21 (m, 2H), 6.99 (d, J=8.0 Hz, 1H), 6.89˜6.83 (m, 3H), 4.90˜4.84 (m, 3H), 4.23˜4.21 (m, 2H), 3.73˜3.71 (m, 2H), 3.54˜3.46 (m, 4H), 3.27˜3.26 (m, 2H), 3.12 (brs, 4H), 2.60 (brs, 4H), 1.39 (s, 9H).

### General Procedure for Preparation of Compound 12

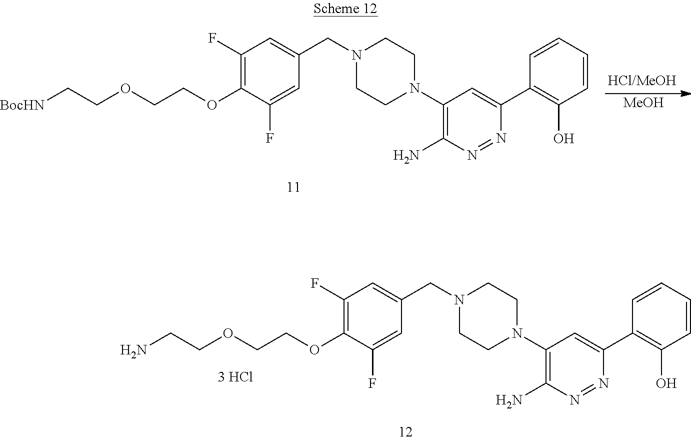 [SMILES: "MeOH" ; CC(C)(C)OC(=O)NCCOCCOc1c(F)cc(CN2CCN(c3cc(-c4ccccc4O)nnc3N)CC2)cc1F ; NCCOCCOc1c(F)cc(CN2CCN(c3cc(-c4ccccc4O)nnc3N)CC2)cc1F]

To a solution of compound 11 (880 mg, 1.42 mmol, 1.00 eq) in MeOH (10.0 mL) was added HCl/MeOH (4 M, 3.66 mL, 10.0 eq) at 20° C., the mixture was stirred at 20° C. for 12 hrs (Scheme 12). LCMS showed the reaction mixture was consumed completely. The mixture was concentrated under reduced pressure to give a residue and then triturated with MTBE (15.0 mL) at 20° C. for 30 mins. The mixture was filtered, and the cake was collected, dried in vacuum to afford compound 12 (300 mg, 0.49 mmol, 33.4% yield, 98.6% purity) as a light yellow solid.

LCMS: Rₜ=0.966 min, m/z: 501 (M+H]⁺.

HPLC: Rₜ=3.19 min.

¹H NMR: (400 MHz, DMSO-d₆)

δ 12.3 (s, 1H), 8.09 (brs, 3H), 7.61˜7.56 (m, 5H), 7.38 (t, J=7.2 Hz, 1H), 7.11 (d, J=8.0 Hz, 1H), 6.97 (t, J=7.2 Hz, 1H), 4.36 (brs, 2H), 4.32˜4.30 (m, 2H), 4.77˜4.75 (m, 4H), 3.68˜3.66 (m, 2H), 3.61˜3.42 (m, 8H), 3.96˜3.92 (m, 2H).

### General Procedure for Preparation of Compound 104

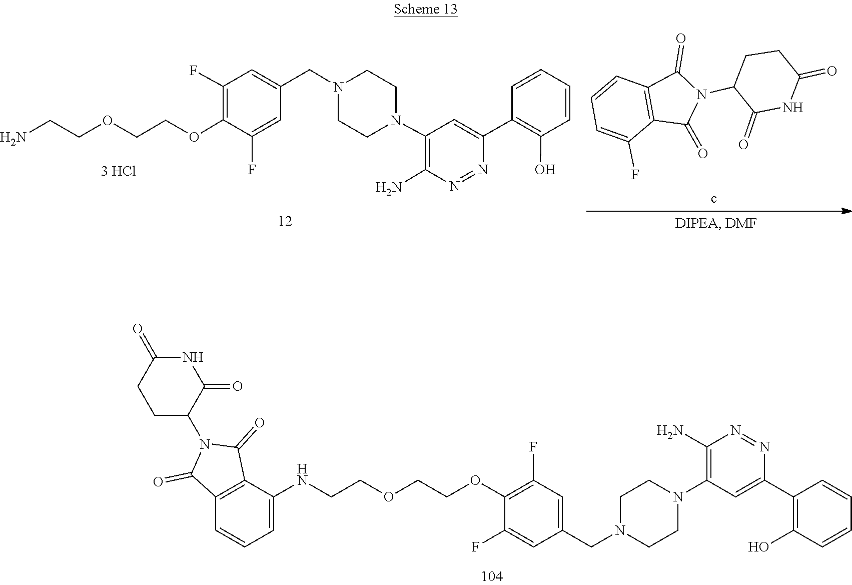

A solution of compound 12 (300 mg, 492 μmol, 1.00 eq, 3HCl), compound c (136 mg, 492 μmol, 1.00 eq) and DIPEA (381 mg, 2.95 μmol, 514 μL, 6.00 eq) in DMF (3.00 mL) was stirred at 100° C. for 14 hrs (Scheme 13).

LCMS showed the reaction mixture was consumed completely. The mixture was filtered and purified by prep-HPLC (column: Waters Xbridge C18 150*50 mm*10 μm; mobile phase: [water(NH₄HCO₃)-ACN]; B %: 38%-68%, 10 min) to afford Compound 104 (114 mg, 145.52 μmol, 29.59% yield, 96.6% purity) as a yellow solid. A total amount of 10 mg of Compound 104 was produced.

LCMS: Rₜ=1.00 min, m/z: 757 (M+H)⁺.

HPLC: Rₜ=3.47 min.

¹H NMR: (400 MHz, CDCl₃)

δ 13.7 (s, 1H), 9.25 (s, 1H), 7.58˜7.56 (m, 1H), 7.49˜7.45 (m, 1H), 7.27˜7.25 (m, 2H), 7.09˜7.03 (m, 2H), 6.93˜6.87 (m, 4H), 6.49 (t, J=5.6 Hz, 1H), 4.95˜4.89 (m, 3H), 4.32˜4.30 (m, 2H), 3.85˜3.78 (m, 4H), 3.55˜3.45 (m, 4H), 3.15 (s, 4H), 2.90˜2.65 (m, 7H), 2.13˜2.09 (m, 1H).

### Example 3—Preparation of Compound 105

### General Procedure for Preparation of Compound 14

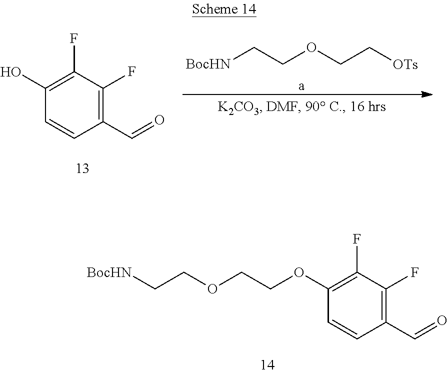

To a solution of compound a (2.73 g, 7.59 mmol, 1.20 eq) in DMF (35.0 mL) was added compound 13 (1.00 g, 6.33 mmol, 1.00 eq) and K₂CO₃ (2.19 g, 15.8 mmol, 2.50 eq), the mixture was stirred at 90° C. for 16 hrs (Scheme 14). LCMS showed the reaction mixture was consumed completely. The reaction mixture was diluted with H₂O (60.0 mL) and extracted with EtOAc (30.0 mL×2). The combined organic layers were washed with brine (80.0 mL×3), dried over Na₂SO₄, filtered and concentrated under reduced pressure to give a residue. The residue was purified by column chromatography (SiO₂, Petroleum ether/Ethyl acetate=5:1 to 0:1) to afford compound 14 (1.00 g, 2.88 mmol, 45.6% yield, 99.5% purity) as a yellow oil.

LCMS: Rₜ=0.993 min, m/z=246.2 (M−Boc+1)

HPLC: Rₜ=2.99 min

¹H NMR: (400 MHz, CDCl₃)

δ 10.2 (s, 1H), 7.60˜7.65 (m, 1H), 6.86˜6.90 (m, 1H), 4.87 (s, 1H), 4.29 (t, J=4.8 Hz, 2H), 3.88 (t, J=4.4 Hz, 2H), 3.62 (t, J=5.2 Hz, 2H), 3.34˜3.35 (m, 2H), 1.45 (s, 9H).

### General Procedure for Preparation of Compound 15

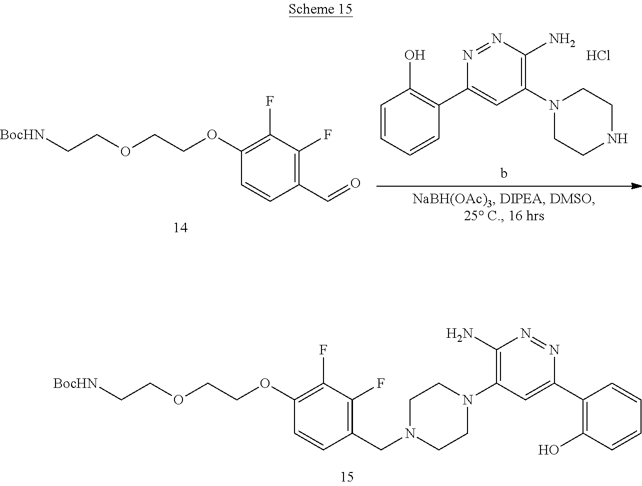 [SMILES: CC(C)(C)OC(=O)NCCOCCOc1ccc(C=O)c(F)c1F ; Nc1nnc(-c2ccccc2O)cc1N1CCNCC1 ; Cl ; CC(C)(C)OC(=O)NCCOCCOc1ccc(CN2CCN(c3cc(-c4ccccc4O)nnc3N)CC2)c(F)c1F]

To a solution of compound b (891 mg, 2.90 mmol, 1.00 eq, HCl) in DMSO (5.00 mL) was added DIPEA (374 mg, 2.90 mmol, 504 μL, 1.00 eq) and the mixture was stirred at 25° C. for 15 min (Scheme 15). Then to the mixture was added a solution of compound 14 (1.00 g, 2.90 mmol, 1.00 eq) in DMSO (5.00 mL) and NaBH(OAc)₃ (1.23 g, 5.79 mmol, 2.00 eq) and the mixture was stirred at 25° C. for 16 hrs. LCMS showed the reaction mixture was consumed completely. The reaction mixture was adjusted to pH=7-8 with NaHCO₃, diluted with H₂O (30.0 mL) and extracted with EtOAc (20.0 mL×2). The combined organic layers were washed with brine (50.0 mL×3), dried over Na₂SO₄, filtered and concentrated under reduced pressure to give a residue. The residue was purified by column chromatography (SiO₂, Petroleum ether/Ethyl acetate=3:1 to 0:1) to afford compound 15 (900 mg, 1.43 mmol, 49.3% yield, 95.3% purity) as a yellow solid.

LCMS: Rₜ=1.04 min, m/z=601.3 (M+1)

HPLC: Rₜ=3.55 min

¹H NMR: (400 MHz, CDCl₃)

δ 7.58 (dd, J=8.0, 1.2 Hz, 1H), 7.29˜7.32 (m, 2H), 7.02˜7.07 (m, 2H), 6.89˜6.93 (m, 1H), 6.75˜6.80 (m, 1H), 4.93 (s, 1H), 4.81 (s, 2H), 4.22 (t, J=4.4 Hz, 2H), 3.85 (t, J=4.8 Hz, 2H), 3.66 (s, 2H), 3.63 (t, J=5.2 Hz, 2H), 3.34˜3.36 (m, 2H), 3.17 (s, 4H), 2.69 (s, 4H), 1.45 (s, 9H).

### General Procedure for Preparation of Compound 16

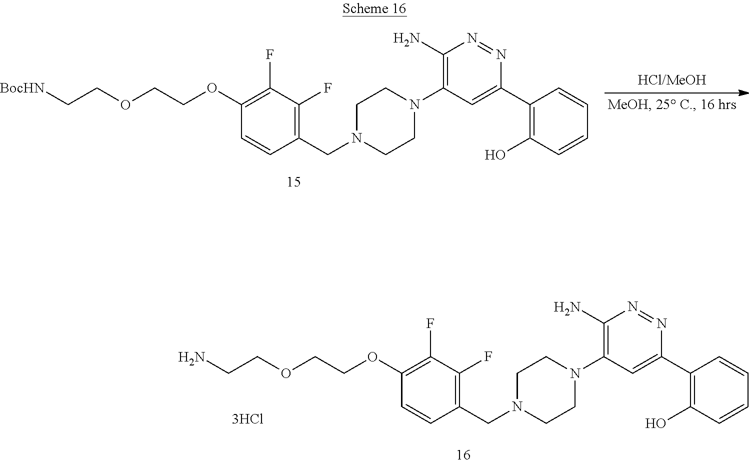 [SMILES: CC(C)(C)OC(=O)NCCOCCOc1ccc(CN2CCN(c3cc(-c4ccccc4O)nnc3N)CC2)c(F)c1F ; NCCOCCOc1ccc(CN2CCN(c3cc(-c4ccccc4O)nnc3N)CC2)c(F)c1F]

To a solution of compound 15 (900 mg, 1.50 mmol, 1.00 eq) in MeOH (10.0 mL) as added HCl/MeOH (4 M, 3.75 mL, 10.0 eq), the mixture was stirred at 20° C. for 16 hrs (Scheme 16). LCMS showed the reaction mixture was consumed completely. The reaction mixture was concentrated under reduced pressure to remove MeOH. The crude product was triturated with MTBE (5V) at 20° C. for 30 min and filtered to afford compound 16 (860 mg, 1.38 mmol, 92.4% yield, 98.2% purity, 3HCl) as a light yellow solid.

LCMS: Rₜ=0.994 min, m/z=501.2 (M+1)

HPLC: Rₜ=3.22 min

¹H NMR: (400 MHz, DMSO-d₆)

δ 12.2 (s, 1H), 8.20 (s, 3H), 7.58˜7.62 (m, 3H), 7.38 (t, J=7.6 Hz, 1H), 7.20 (d, J=8.0 Hz, 1H), 7.15 (d, J=8.0 Hz, 1H), 6.97 (t, J=7.6 Hz, 1H), 4.39 (s, 2H), 4.30˜4.32 (m, 2H), 3.69˜3.84 (m, 7H), 3.39˜3.50 (m, 7H), 2.94˜2.98 (m, 2H).

### General Procedure for Preparation of Compound 105

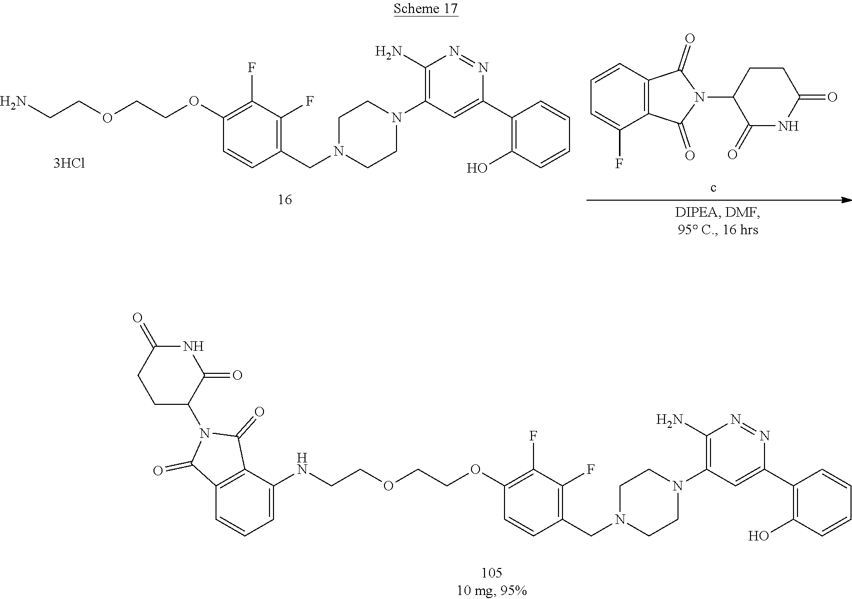

To a solution of compound 16 (300 mg, 492 μmol, 1.00 eq, 3HCl) in DMF (3.00 mL) was added DIPEA (381 mg, 2.95 mmol, 514 μL, 6.00 eq) and compound c (136 mg, 492 μmol, 1.00 eq) (Scheme 17). The mixture was stirred at 95° C. for 16 hrs. LCMS showed the reaction mixture was consumed completely. The reaction mixture was filtered, and purified by prep-HPLC (neutral condition:column: Waters Xbridge C18 150*50 mm*10 um; mobile phase: [water(NH₄HCO₃)-ACN]; B %: 39%-69%, 10 min) to afford Compound 105 (105 mg, 138 μmol, 28.1% yield, 99.7% purity) as a yellow solid. A total amount of 10 mg of Compound 105 at a purity of 95% was produced.

LCMS: Rₜ=0.994 min, m/z=757.3 (M+1)

HPLC: Rₜ=3.380 min

¹H NMR: (400 MHz, CDCl₃)

δ 13.7 (s, 1H), 10.1 (s, 1H), 7.58 (d, J=8.0 Hz, 1H), 7.47 (t, J=8.0 Hz, 1H), 7.28 (s, 2H), 7.06 (dd, J=16.4, 7.2 Hz, 2H), 7.00 (t, J=7.2 Hz, 1H), 6.88˜6.92 (m, 2H), 6.84 (t, J=7.6 Hz, 1H), 6.55 (t, J=5.3 Hz, 1H), 4.87˜4.92 (m, 3H), 4.23˜4.31 (m, 2H), 3.91 (t, J=4.0 Hz, 2H), 3.84 (t, J=5.2 Hz, 2H), 3.72 (d, J=12.8 Hz, 1H), 3.57 (d, J=12.8 Hz, 1H), 3.45˜3.49 (m, 2H), 3.16 (s, 4H), 2.68˜2.96 (m, 7H), 2.10˜2.15 (m, 1H).

### Example 4—Preparation of Compound 106

### General Procedure for Preparation of Compound 18

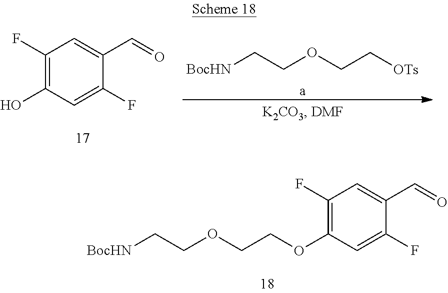

To a solution of compound a (2.73 g, 7.60 mmol, 1.20 eq) and compound 17 (1.00 g, 6.33 mmol, 1.00 eq) in DMF (25.0 mL) was added K₂CO₃ (2.19 g, 15.8 mmol, 2.50 eq) (Scheme 18), the mixture was stirred at 90° C. for 16 hrs (Scheme 15). LCMS showed the reaction mixture was consumed completely. The mixture was diluted with H₂O (100 mL), then extracted with EtOAc (70.0 mL×3). The combined organic layers were washed with brine (70.0 mL×2), dried over Na₂SO₄, filtered and concentrated under reduced pressure to give compound 18 (2.20 g, 6.20 mmol, 98.0% yield, 97.4% purity) as a yellow oil and it was used for next step directly.

LCMS: Rₜ=0.893 min, m/z: 246 (M+H−Boc)⁺.

HPLC: Rₜ=2.84 min.

¹H NMR: (400 MHz, CDCl₃)

δ 10.1 (d, J=2.8 Hz, 1H), 7.53 (dd, J=10.4, 6.4 Hz, 1H), 7.76 (dd, J=11.2, 6.0 Hz, 1H), 4.91 (brs, 1H), 4.24˜4.22 (m, 2H), 3.86˜3.84 (m, 2H), 3.60˜3.58 (m, 2H), 3.32˜3.30 (m, 2H), 1.44 (s, 9H).

### General Procedure for Preparation of Compound 19

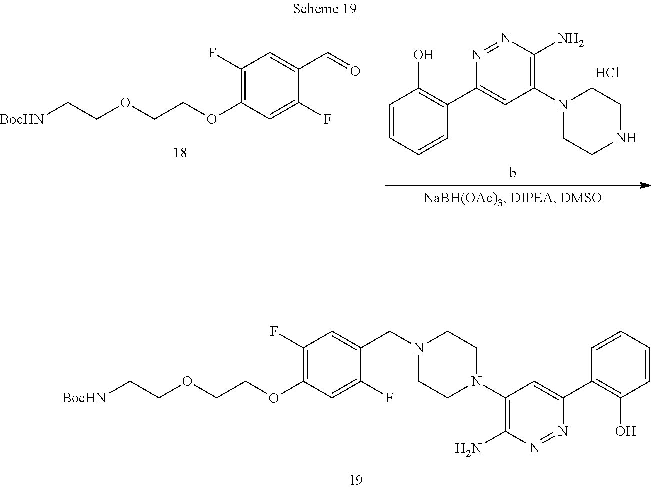 [SMILES: CC(C)(C)OC(=O)NCCOCCOc1cc(F)c(C=O)cc1F ; Nc1nnc(-c2ccccc2O)cc1N1CCNCC1 ; Cl ; CC(C)(C)OC(=O)NCCOCCOc1cc(F)c(CN2CCN(c3cc(-c4ccccc4O)nnc3N)CC2)cc1F]

To a solution of compound b (1.78 g, 5.79 mmol, 1.00 eq, HCl) in DMSO (10.0 mL) was added DIPEA (748.50 mg, 5.79 mmol, 1.01 mL, 1.00 eq), the mixture was stirred at 20° C. for 15 mins (Scheme 19). Then to the mixture was added a solution of compound 18 (2.00 g, 5.79 mmol, 1.0 eq) in DMSO (10.0 mL) and NaBH(OAc)₃ (2.45 g, 11.58 mmol, 2.00 eq), the mixture was stirred at 25° C. for 15 hrs. LCMS showed the reaction mixture was consumed completely. The reaction mixture was diluted with EtOAc (30.0 mL) and adjusted to pH=7˜8 with aq·NaHCO₃ (sat, 30 mL), then H₂O (60.0 mL) was added, extracted with EtOAc (30.0 mL×2). The combined organic layers were washed with brine (30.0 mL×3), filtered and concentrated under reduced pressure to give a residue. The residue was purified by column chromatography (SiO2, Petroleum ether/Ethyl acetate=5/1 to 1/2) to afford compound 19 (1.50 g, 2.45 mmol, 42.3% yield, 98.2% purity) as a yellow solid.

LCMS: Rₜ=1.03 min, m/z: 601 (M+H)⁺.

HPLC: Rₜ=3.56 min.

¹H NMR: (400 MHz, CDCl₃)

δ 13.7 (s, 1H), 7.59 (dd, J=8.0, 1.6 Hz, 1H), 7.33˜7.27 (m, 2H), 7.15 (dd, J=11.6, 6.8 Hz, 1H), 7.06 (dd, J=8.0, 1.2 Hz, 1H), 6.93˜6.89 (m, 1H), 6.73 (dd, J=10.8, 6.8 Hz, 1H), 4.92 (s, 1H), 4.79 (s, 2H), 4.19˜4.16 (m, 2H), 3.86˜3.84 (m, 2H), 3.63˜3.59 (m, 4H), 3.37˜3.34 (m, 2H), 3.18 (brs, 4H), 2.69 (brs, 4H), 1.45 (s, 9H).

### General Procedure for Preparation of Compound 20

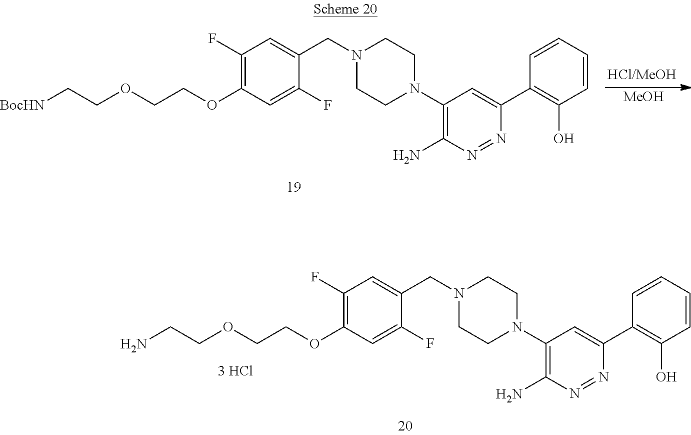 [SMILES: "MeOH" ; CC(C)(C)OC(=O)NCCOCCOc1cc(F)c(CN2CCN(c3cc(-c4ccccc4O)nnc3N)CC2)cc1F ; NCCOCCOc1cc(F)c(CN2CCN(c3cc(-c4ccccc4O)nnc3N)CC2)cc1F]

To a solution of compound 19 (1.40 g, 2.33 mmol, 1.00 eq) in MeOH (14.0 mL) was added HCl/MeOH (4 M, 5.83 mL, 10.0 eq) at 20° C., the mixture was stirred at 20° C. for 12 hrs (Scheme 20). LCMS showed the reaction mixture was consumed completely. The mixture was concentrated under reduced pressure to give a residue and then triturated with MTBE (15.0 mL) at 20° C. for 30 mins. The mixture was filtered, and the cake was collected, dried in vacuum to afford compound 20 (1.34 g, 2.16 mmol, 92.8% yield, 98.5% purity) as a light yellow solid.

LCMS: Rₜ=1.00 min, m/z: 501 (M+H)⁺.

HPLC: Rₜ=3.17 min.

¹H NMR: (400 MHz, MeOH-d₄)

δ 7.71 (brs, 1H), 7.65˜7.63 (m, 1H), 7.58˜7.53 (m, 1H), 7.46˜7.42 (m, 1H), 7.20˜7.15 (m, 1H), 7.07˜7.03 (m, 2H), 4.47 (brs, 2H), 4.32˜4.30 (m, 2H), 4.96˜4.94 (m, 4H), 3.82˜3.80 (m, 2H), 3.72˜3.58 (m, 5H), 3.35 (s, 1H), 3.17˜3.15 (m, 2H).

### General Procedure for Preparation of Compound 106

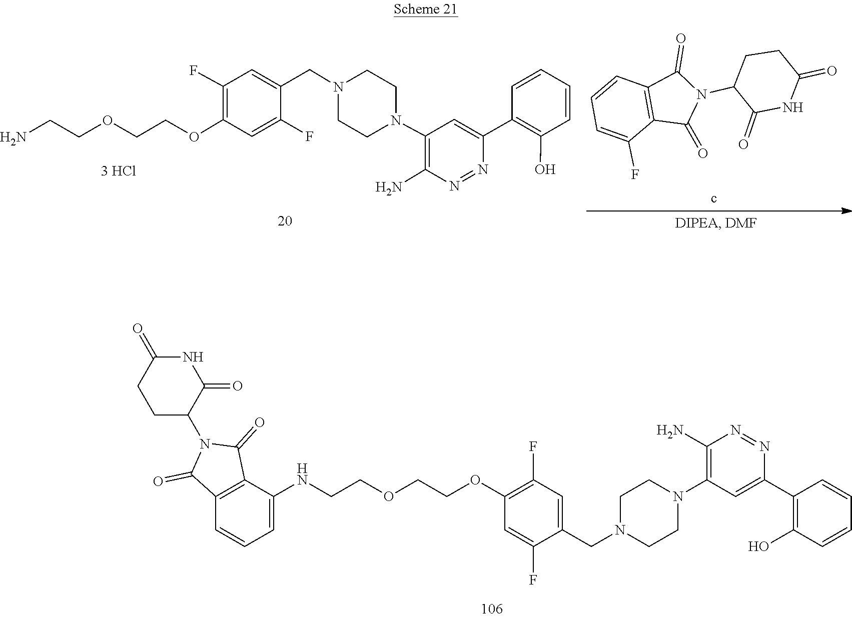

A solution of compound 20 (300 mg, 492 umol, 1.00 eq, 3HCl), compound c (136 mg, 492 μmol, 1.00 eq) and DIPEA (381 mg, 2.95 mmol, 514 μL, 6.00 eq) in DMF (3.00 mL) was stirred at 100° C. for 14 hrs (Scheme 21).

LCMS showed the reaction mixture was consumed completely. The mixture was filtered and purified by prep-HPLC (column: Waters Xbridge C18 150*50 mm*10 μm; mobile phase: [water(NH₄HCO₃)-ACN]; B %: 38%-68%, 10 min) to afford Compound 106 (120 mg, 157 μmol, 31.9% yield, 99.0% purity) as a yellow solid. A total amount of 10 mg of Compound 106 was produced.

LCMS: Rt=1.00 min, m/z: 757 (M+H)⁺.

HPLC: Rt=3.37 min.

¹H NMR: (400 MHz, CDCl₃)

δ 13.7 (s, 1H), 9.83 (s, 1H), 7.58 (d, J=7.6 Hz, 1H), 7.47 (t, J=7.6 Hz, 1H), 7.29˜7.26 (m, 2H), 7.09˜7.03 (m, 2H), 6.92˜6.88 (m, 2H), 6.78˜6.74 (m, 1H), 6.54 (t, J=5.2 Hz, 3H), 4.91˜4.86 (m, 3H), 4.21˜4.20 (m, 2H), 3.95˜3.82 (m, 4H), 3.66˜3.63 (m, 1H), 3.54˜3.44 (m, 3H), 3.16 (brs, 4H), 2.92˜2.70 (m, 7H), 2.13˜2.09 (m, 1H).

### Example 5—Preparation of Compound 107

### General Procedure for Preparation of Compound 22

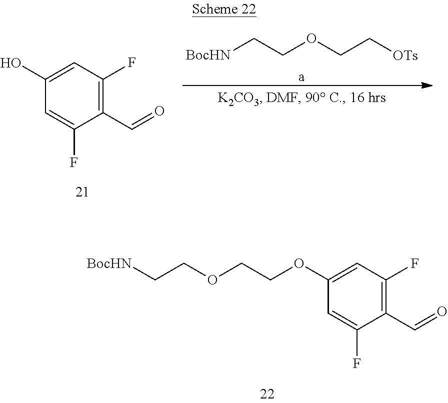

To a solution of compound a (2.50 g, 6.96 mmol, 1.10 eq) in DMF (25.0 mL) was added compound 21 (1.00 g, 6.33 mmol, 1.00 eq) and K₂CO₃ (2.19 g, 15.8 mmol, 2.50 eq) (Scheme 22). The mixture was stirred at 90° C. for 16 hrs (Scheme 19). HPLC showed the reaction mixture was consumed completely. The reaction mixture was diluted with H₂O (50.0 mL) and extracted with EtOAc (30.0 mL×2). The combined organic layers were washed with brine (60.0 mL×3), dried over Na₂SO₄, filtered and concentrated under reduced pressure to give a residue. The residue was purified by column chromatography (SiO₂, Petroleum ether/Ethyl acetate=5:1 to 1:1) to afford compound 22 (1.90 g, 5.42 mmol, 85.7% yield, 98.5% purity) as a yellow oil.

LCMS: Rₜ=0.896 min, m/z=245.9 (M−Boc)

HPLC: Rₜ=2.889 min

¹H NMR: (400 MHz, CDCl₃)

δ 10.2 (s, 1H), 6.53 (d, J=10.8 Hz, 2H), 4.88 (s, 1H), 4.17 (t, J=4.4 Hz, 2H), 3.84 (t, J=4.4 Hz, 2H), 3.61 (t, J=5.2 Hz, 2H), 3.33˜3.38 (m, 2H), 1.45 (s, 9H).

### General Procedure for Preparation of Compound 23

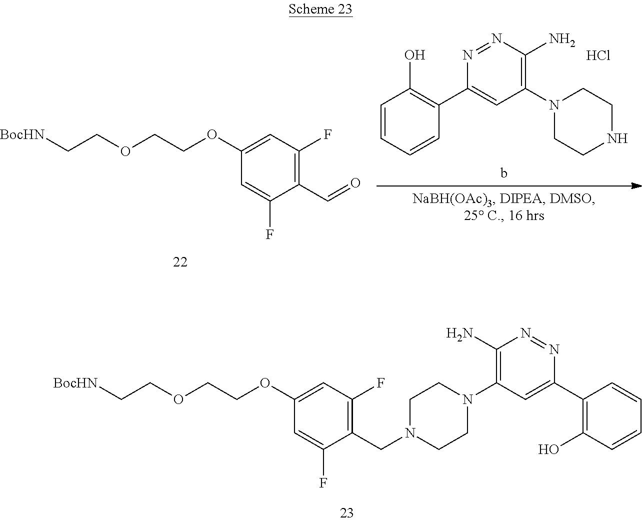 [SMILES: CC(C)(C)OC(=O)NCCOCCOc1cc(F)c(C=O)c(F)c1 ; Nc1nnc(-c2ccccc2O)cc1N1CCNCC1 ; Cl ; CC(C)(C)OC(=O)NCCOCCOc1cc(F)c(CN2CCN(c3cc(-c4ccccc4O)nnc3N)CC2)c(F)c1]

To a solution of compound b (1.69 g, 5.50 mmol, 1.00 eq, HCl) in DMSO (10.0 mL) was added DIPEA (711 mg, 5.50 mmol, 958 μL, 1.00 eq) and the mixture was stirred at 20° C. for 15 min (Scheme 23). Then to the mixture was added a solution of compound 22 (1.90 g, 5.50 mmol, 1.00 eq) in DMSO (10.0 mL) and NaBH(OAc)₃ (2.33 g, 11.0 mmol, 2.00 eq) and the mixture was stirred at 20° C. for 16 hrs. LCMS showed the reaction mixture was consumed completely. The reaction mixture was adjusted to pH=7-8 with NaHCO₃, diluted with H₂O (60.0 mL) and extracted with EtOAc (30.0 mL×2). The combined organic layers were washed with brine (60.0 mL×3), dried over Na₂SO₄, filtered and concentrated under reduced pressure to give a residue. The residue was purified by column chromatography (SiO₂, Petroleum ether/Ethyl acetate=3:1 to 0:1) to afford compound 23 (1.90 g, 3.08 mmol, 55.9% yield, 97.3% purity) as a yellow solid.

LCMS: Rₜ=1.02 min, m/z=601.3 (M+1)

HPLC: Rₜ=3.58 min

¹H NMR: (400 MHz, CDCl₃)

δ 13.8 (s, 1H), 7.57 (dd, J=8.0, 1.2 Hz, 1H), 7.28˜7.30 (m, 2H), 7.04˜7.06 (m, 1H), 6.89˜6.93 (m, 1H), 6.52 (d, J=9.2 Hz, 2H), 4.94 (s, 1H), 4.81 (s, 2H), 4.10 (t, J=4.4 Hz, 2H), 3.82 (t, J=4.8 Hz, 2H), 3.74 (s, 2H), 3.61 (t, J=5.2 Hz, 2H), 3.35˜3.36 (m, 2H), 3.16 (s, 4H), 2.70 (s, 4H), 1.46 (s, 9H)

### General Procedure for Preparation of Compound 24

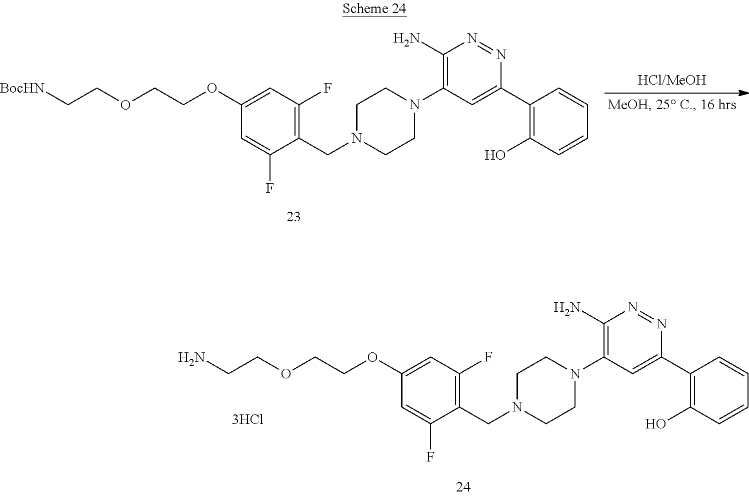 [SMILES: CC(C)(C)OC(=O)NCCOCCOc1cc(F)c(CN2CCN(c3cc(-c4ccccc4O)nnc3N)CC2)c(F)c1 ; NCCOCCOc1cc(F)c(CN2CCN(c3cc(-c4ccccc4O)nnc3N)CC2)c(F)c1]

To a solution of compound 23 (1.90 g, 3.16 mmol, 1.00 eq) in MeOH (20.0 mL) was added HCl/MeOH (4 M, 7.91 mL, 10.0 eq) (Scheme 24). The mixture was stirred at 20° C. for 16 hrs. LCMS showed the reaction mixture was consumed completely. The reaction mixture was concentrated under reduced pressure to remove MeOH. The crude product was triturated with MTBE (5V) at 20° C. for 30 min and filtered to afford compound 24 (1.70 g, 2.73 mmol, 86.3% yield, 97.9% purity, 3HCl) as a light yellow solid.

LCMS: Rₜ=0.977 min, m/z=501.2 (M+1)

HPLC: Rₜ=3.196 min

¹H NMR: (400 MHz, MeOH-d₄)

δ 7.73 (s, 1H), 7.65 (dd, J=8.0, 1.6 Hz, 1H), 7.42˜7.46 (m, 1H), 7.03˜7.07 (m, 2H), 6.84 (d, J=9.6 Hz, 2H), 4.52 (s, 2H), 4.26 (t, J=4.4 Hz, 2H), 3.97 (s, 1H), 3.91 (t, J=4.4 Hz, 2H), 3.79 (t, J=4.8 Hz, 2H), 3.73 (s, 2H), 3.60˜3.63 (m, 2H), 3.43 (s, 2H), 3.35 (s, 1H), 3.16 (t, J=4.8 Hz, 2H).

### General Procedure for Preparation of Compound 107

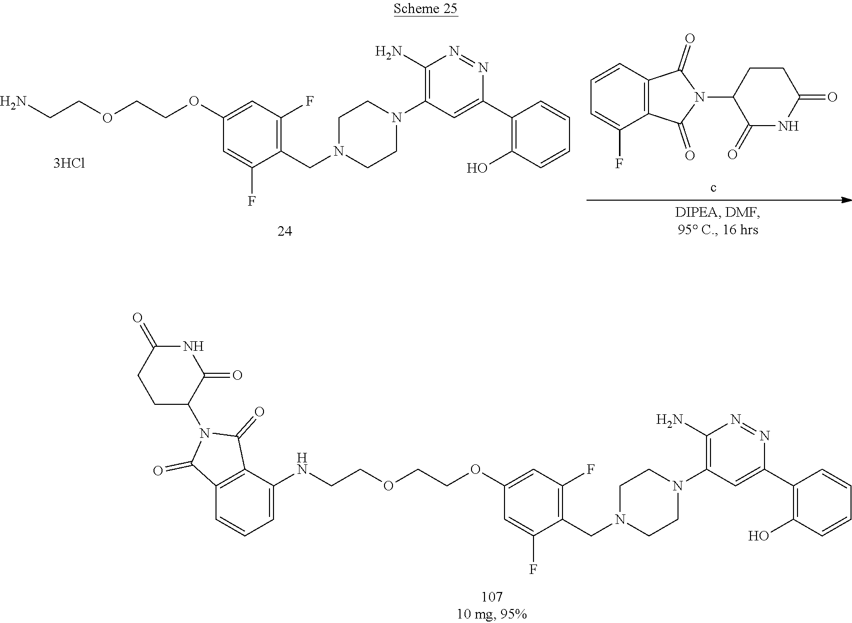

To a solution of compound 24 (300 mg, 492 μmol, 1.00 eq, 3HCl) in DMF (3.00 mL) was added DIPEA (381 mg, 2.95 mmol, 514 μL, 6.00 eq) and compound c (136 mg, 492 μmol, 1.00 eq) (Scheme 25). The mixture was stirred at 95° C. for 16 hrs. LCMS (EW15912-251-P1A) showed the reaction mixture was consumed completely. The reaction mixture was filtered and purified by prep-HPLC (neutral condition:column: Waters Xbridge C18 150*50 mm*10 μm; mobile phase: [water(NH₄HCO₃)-ACN]; B%: 38%-68%, 10 min) to afford Compound 107 (100 mg, 132 μmol, 26.9% yield) as a yellow solid. A total amount of 10 mg of Compound 107 at a purity of 95% was produced.

LCMS: Rₜ=1.01 min, m/z=757.3 (M+1)

HPLC: Rₜ=3.40 min

¹H NMR: (400 MHz, CDCl₃)

δ 13.8 (s, 1H), 10.1 (s, 1H), 7.58 (d, J=7.2 Hz, 1H), 7.47 (t, J=8.0 Hz, 1H), 7.29 (s, 1H), 7.25 (s, 1H), 7.09 (d, J=7.2 Hz, 1H), 7.04 (d, J=8.4 Hz, 1H), 6.89˜6.92 (m, 2H), 6.51˜6.57 (m, 3H), 4.88˜4.92 (m, 3H), 4.13 (t, J=3.6 Hz, 2H), 3.75˜3.89 (m, 5H), 3.61 (d, J=13.2 Hz, 1H), 3.44˜3.48 (m, 2H), 3.18 (s, 4H), 2.70˜2.93 (m, 7H), 2.08˜2.12 (m, 1H).

### Example 6—Preparation of Compound 108

### General Procedure for Preparation of Compound 2A

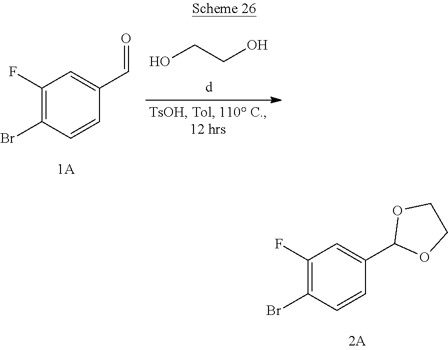

A mixture of compound 1A (20.0 g, 98.5 mmol, 1.00 eq.), compound d (18.3 g, 296 mmol, 16.5 mL, 3.00 eq.) (Scheme 23), TsOH (339 mg, 1.97 mmol, 0.02 eq.) in Tol. (200 mL) was degassed and purged with N2 for 3 times, and stirred at 110° C. for 23 hrs under N2 atmosphere (Scheme 26). TLC (PE:EtOAc=10:1, R1 R_{f}=0.57, P1 Rf=0.51) showed compound 1A was remained and one new spot was detected. The mixture reaction was cooled to room temperature, was purified by a pad of silica with EtOAc to afford compound 2A (10.8 g, 43.4 mmol, 99.2% purity) as a yellow liquid.

LCMS: Rₜ=0.920 min, m/z=280 (M+Na)⁺

HPLC: Rₜ=2.44 min

¹H NMR: (400 MHz, DMSO-d₆)

δ 7.70˜7.66 (m, 1H), 7.37 (dd, J=9.6, 1.6 Hz, 1H), 7.21 (dd, J=8.4, 1.6 Hz, 1H), 5.74 (s, 1H), 4.0˜3.94 (m, 4H).

### General Procedure for Preparation of Compound 3A

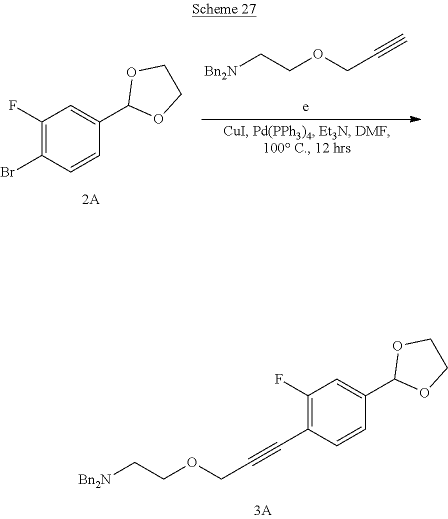

To a solution of compound 2A (2.00, 8.11 mmol, 1.00 eq.), compound e (3.40 g, 12.2 mmol, 1.50 eq.) and Et₃N (2.46 g, 24.3 mmol, 3.39 mL, 3.00 eq.) in DMF (20 mL) was added CuI (309 mg, 1.62 mmol, 0.20 eq.) and Pd(PPh₃)₄ (2.81 g, 2.43 mmol, 0.30 eq.) was degassed and purged with N2 for 3 times, and then the mixture was stirred at 100° C. for 12 hrs under N2 atmosphere (Scheme 27). LC-MS showed the reaction mixture was consumed completely. The mixture reaction was filtered and the filtrate was concentrated. The residue was purified by column chromatography (SiO₂, Petroleum ether/Ethyl acetate=30/1) and further purified by reversed-phase HPLC (0.1% NH₃·H₂O) to afford compound 3A (583 mg, 1.28 mmol, 15.8% yield, 97.9% purity) as a yellow oil.

LCMS: Rₜ=0.912 min, m/z=446 (M+H)⁺

HPLC: Rₜ=2.02 min

¹H NMR: (400 MHz, CDCl₃)

δ 7.41˜7.18 (m, 13H), 5.82 (s, 1H), 4.37 (s, 2H), 4.13˜4.05 (m, 4H), 3.73 (t, J=6.0 Hz, 2H), 3.67 (s, 4H), 2.77 (t, J=6.0 Hz, 2H).

### General Procedure for Preparation of Compound 4A

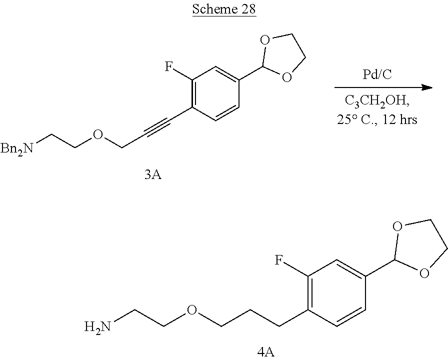

To a solution of compound 3A (473 mg, 1.06 mmol, 1.00 eq.) in CF₃CH₂OH (5.00 mL) was added Pd/C (400 mg, 10% purity) was degassed and purged with H₂ for 3 times, and then the mixture was stirred at 40° C. for 12 hrs under H₂ atmosphere (Scheme 25). LC-MS showed the reaction mixture was consumed completely. The reaction mixture was filtered and the cake was washed with MeOH, the filtrate was concentrated under reduced pressure to afford compound 4A (290 mg, crude) as a yellow oil.

LCMS: Rₜ=0.818 min, m/z=270 (M+H)⁺

HPLC: Rₜ=1.72 min

¹H NMR: (400 MHz, CDCl₃)

δ 7.23˜7.14 (m, 3H), 5.78 (s, 1H), 4.14˜4.01 (m, 4H), 3.47˜3.43 (m, 2H), 2.84 (t, J=6.8 Hz, 2H), 2.74 (t, J=7.6 Hz, 2H), 1.93˜1.86 (m, 2H).

### General Procedure for Preparation of Compound 5A

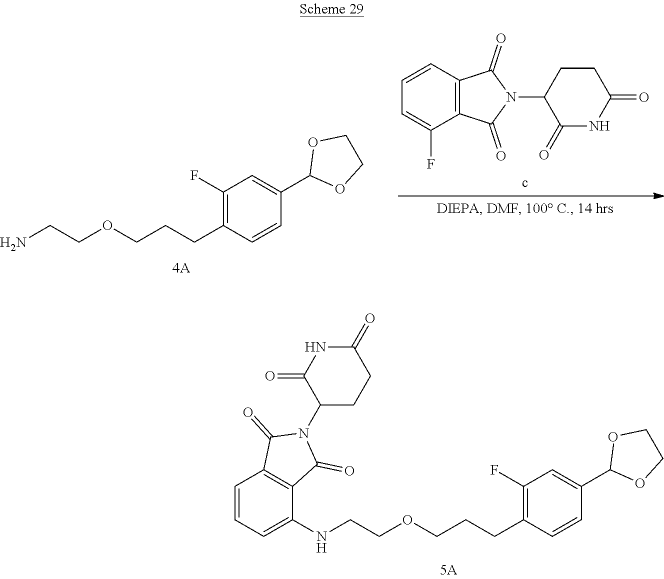 [SMILES: NCCOCCCc1ccc(C2OCCO2)cc1F ; O=C1CCC(N2C(=O)c3cccc(F)c3C2=O)C(=O)N1 ; O=C1CCC(N2C(=O)c3cccc(NCCOCCCc4ccc(C5OCCO5)cc4F)c3C2=O)C(=O)N1]

A solution of compound 4A (284 mg, 1.05 mmol, 1.00 eq), compound c (291 mg, 1.05 mmol, 1.00 eq), DIPEA (409 mg, 3.16 mmol, 551 μL, 3.00 eq) in DMF (3.00 mL) was stirred at 100° C. for 14 hrs (Scheme 26). LC-MS showed the reaction mixture was consumed completely. EtOAc (10 mL) and H₂O (10 mL) was added, the resulting mixture was extracted with EtOAc 20 mL (10 mL×2), the combined organic layers were washed with brine 20 mL (10 mL×2), dried over Na₂SO₄ filtered and concentrated under reduced pressure to give a residue. The residue was purified by column chromatography (SiO₂, Petroleum ether/Ethyl acetate=5/1 to 1/1 and 2% DCM) to afford compound 5A (290 mg, 552 μmol, 52.3% yield) as a yellow solid.

LCMS: Rₜ=0.973 min, m/z=526 (M+H)⁺

HPLC: Rₜ=2.60 min

¹H NMR: (400 MHz, CDCl₃)

δ 8.58 (s, 1H), 7.51˜7.47 (m, 1H), 7.20˜7.09 (m, 4H), 6.93 (t, J=8.4 Hz, 1H), 6.53 (t, J=5.2 Hz, 1H), 5.76 (s, 1H), 4.91˜4.87 (m, 1H), 4.13˜4.01 (m, 4H), 3.47˜3.44 (m, 1H), 3.65˜3.63 (m, 3H), 2.88˜2.69 (m, 5H), 2.11˜2.07 (m, 1H), 1.92˜1.85 (m, 4H), THE was contained.

### General Procedure for Preparation of Compound 6A

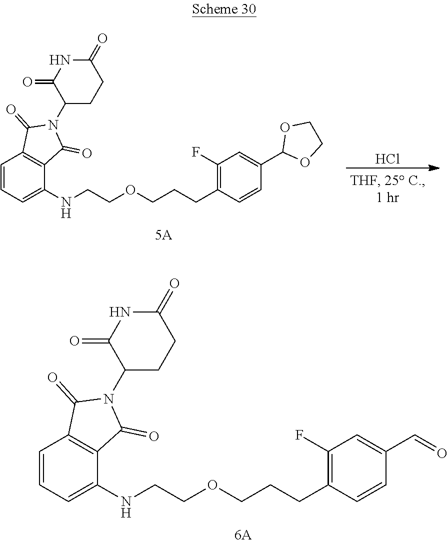 [SMILES: O=C1CCC(N2C(=O)c3cccc(NCCOCCCc4ccc(C5OCCO5)cc4F)c3C2=O)C(=O)N1 ; Cl ; O=Cc1ccc(CCCOCCNc2cccc3c2C(=O)N(C2CCC(=O)NC2=O)C3=O)c(F)c1]

To a solution of compound 5A (145 mg, 276 μmol, 1.00 eq.) in THF (1.00 mL) was added HCl (0.5 M, 1.00 mL), the mixture was stirred at 40° C. for 1 hr (Scheme 27). LC-MS showed the reaction mixture was consumed completely. The mixture was adjusted to pH=8 with aq·NaHCO₃ (sat) and then EtOAc (10 mL) and H₂O (10 mL) was added. The resulting mixture was extracted with EtOAc 20 mL (10 mL×2), dried over Na₂SO₄ filtered and concentrated under reduced pressure to afford compound 6A (103 mg, 200 μmol, 72.6% yield, 93.7% purity) as a yellow solid.

### General Procedure for Preparation of Compound 108

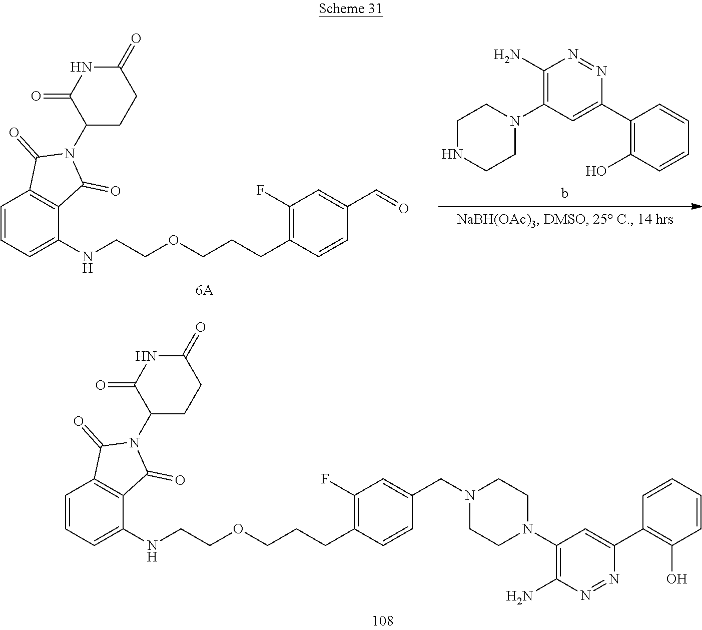 [SMILES: O=Cc1ccc(CCCOCCNc2cccc3c2C(=O)N(C2CCC(=O)NC2=O)C3=O)c(F)c1 ; Nc1nnc(-c2ccccc2O)cc1N1CCNCC1 ; Nc1nnc(-c2ccccc2O)cc1N1CCN(Cc2ccc(CCCOCCNc3cccc4c3C(=O)N(C3CCC(=O)NC3=O)C4=O)c(F)c2)CC1]

To a solution of compound b (65.8 mg, 214 μmol, 1.00 eq., HCl) in DMSO (1.00 mL) was added DIPEA (27.6 mg, 214 μmol, 37.26 μL, 1.00 eq.), the mixture was stirred at 20° C. for 15 mins (Scheme 28). Then compound 6A (103 mg, 214 μmol, 1.00 eq) and NaBH(OAc)₃ (90.7 mg, 428 μmol, 2.00 eq) was added, the mixture was stirred at 25° C. for 14 hrs. LC-MS showed 51.9% of desired mass was detected. The reaction mixture was filtered and purified by prep-HPLC (column: Waters Xbridge 150*25 mm*5 μm; mobile phase: [water (ammonia hydroxide v/v)-ACN]; B %: 40%-70%, 9 min) to afford Compound 108 (28.0 mg, 27.8% yield) as a yellow solid.

LCMS: Rₜ=1.02 min, m/z=737 (M+H)⁺

HPLC: Rₜ=3.93 min

¹H NMR: (400 MHz, CDCl₃)

δ 13.7 (s, 1H), 9.53 (s, 1H), 7.57 (d, J=7.6 Hz, 1H), 7.50˜7.47 (m, 1H), 7.28˜7.24 (m, 2H), 7.20˜7.16 (m, 1H), 7.09 (d, J=6.8 Hz, 1H), 7.05˜7.00 (m, 3H), 6.93˜9.86 (m, 2H), 6.54 (t, J=5.6 Hz, 1H), 5.03 (s, 2H), 4.94˜4.90 (m, 1H), 3.68˜3.46 (m, 8H), 3.15 (s, 4H), 2.77˜2.66 (m, 9H), 2.14˜2.09 (m, 1H), 1.94˜1.90 (m, 2H).

### Example 7—Preparation of Compound 102

### General Procedure for Preparation of Compound 26

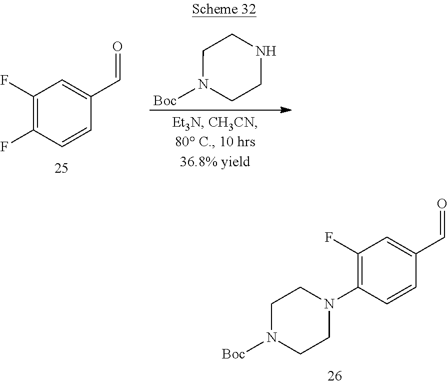 [SMILES: O=Cc1ccc(F)c(F)c1 ; CC(C)(C)OC(=O)N1CCNCC1 ; CC(C)(C)OC(=O)N1CCN(c2ccc(C=O)cc2F)CC1]

To a solution of compound 25 (1.00 g, 7.04 mmol, 763 uL) in CH₃CN (20.0 mL) was added Et₃N (1.42 g, 14.0 mmol, 1.96 mL) and tert-butyl piperazine-1-carboxylate (1.57 g, 8.44 mmol) (Scheme 32). The mixture was stirred at 80° C. for 10 hrs. TLC (Petroleum ether:Ethyl acetate=5:1) showed some of the starting material remained was and a main spot (Rf=0.25) was formed. The reaction mixture was filtered and the filter liquor was concentrated to give the crude product. The residue was purified by column chromatography (SiO₂, Petroleum ether/Ethyl acetate=30/1 to 5/1) to give compound 26 (0.80 g, 2.59 mmol, 36.8% yield) as light yellow oil.

LCMS: Rₜ=0.963 min, m/z=253.1 (M+H)⁺

### General Procedure for Preparation of Compound 27

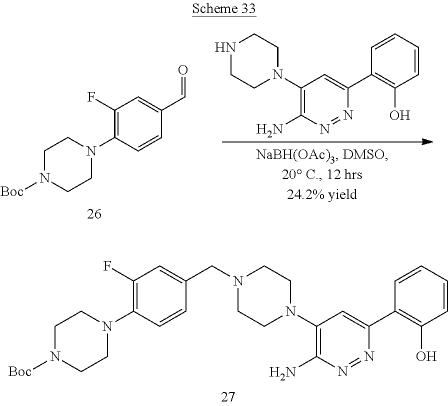 [SMILES: CC(C)(C)OC(=O)N1CCN(c2ccc(C=O)cc2F)CC1 ; Nc1nnc(-c2ccccc2O)cc1N1CCNCC1 ; CC(C)(C)OC(=O)N1CCN(c2ccc(CN3CCN(c4cc(-c5ccccc5O)nnc4N)CC3)cc2F)CC1]

To a solution of compound 26 (1.00 g, 3.24 mmol) in DMSO (10.0 mL) was added 2-(6-amino-5-piperazin-1-yl-pyridazin-3-yl) phenol (967 mg, 3.57 mmol, HCl salt), NaBH(OAc)₃ (824 mg, 3.89 mmol) and Et₃N (656 mg, 6.49 mmol, 902 uL) (Scheme 33). The mixture was stirred at 20° C. for 12 hrs. TLC (Petroleum ether:Ethyl acetate=1:1, P1:Rf=0.25) showed the start material was remained and some new spots was detected. The reaction mixture was diluted with H₂O (30 mL), and then extracted with DCM (20 mL×2). The combined organic layers were washed with brine (10 mL*2), dried over Na₂SO₄, filtered and concentrated under reduced pressure to give a residue. The residue was purified by column chromatography (SiO2, Petroleum ether/Ethyl acetate=10/1 to 2/1) to afford compound 27 (450 mg, 784 umol, 24.2% yield, 98.3% purity) as a white solid.

LCMS: Rₜ=0.805 min, m/z=564.4 (M+H)⁺

### General Procedure for Preparation of Compound 28

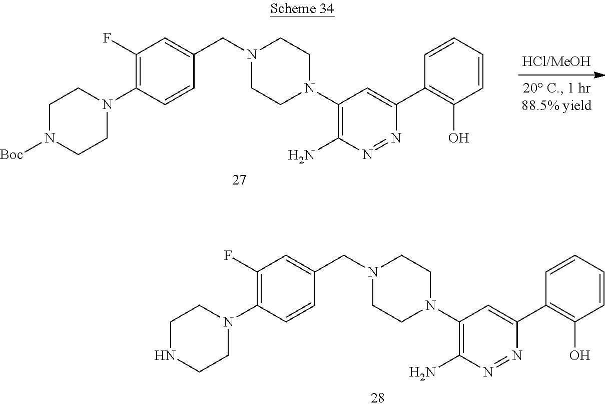 [SMILES: CC(C)(C)OC(=O)N1CCN(c2ccc(CN3CCN(c4cc(-c5ccccc5O)nnc4N)CC3)cc2F)CC1 ; Nc1nnc(-c2ccccc2O)cc1N1CCN(Cc2ccc(N3CCNCC3)c(F)c2)CC1]

To a solution of compound 27 (400 mg, 709 umol) in MeOH (4.00 mL) was added HCl/MeOH (4 M, 4.00 mL) (Scheme 34). Then the mixture was stirred at 20° C. for 1 hr. The mixture was concentrated under reduced pressure to give a residue. The mixture was suspended in DCM (10 mL) and then adjust pH to 8-9 with saturated NaHCO3 solution. Then the mixture was extracted with DCM (20.0 mL×2). The combined organic layers were washed with brine (10 mL), dried over Na₂SO₄, filtered and concentrated under reduced pressure to afford compound 28 (300 mg, 628 umol, 88.5% yield, 97.1% purity) as a white solid.

LCMS: Rₜ=1.033 min, m/z=464.1 (M+H)⁺

¹H NMR: (400 MHz, DMSO-d₆)

δ 14.4-14.2 (m, 1H), 8.0-7.9 (m, 1H), 7.51 (s, 1H), 7.30-7.21 (m, 1H), 7.09-7.03 (m, 2H), 7.01-6.95 (m, 1H), 6.95-6.82 (m, 2H), 6.24 (s, 2H), 3.49 (s, 2H), 3.11 (s, 4H), 3.02-2.98 (m, 8H), 2.58 (s, 5H)

### General Procedure for Preparation of Compound 102

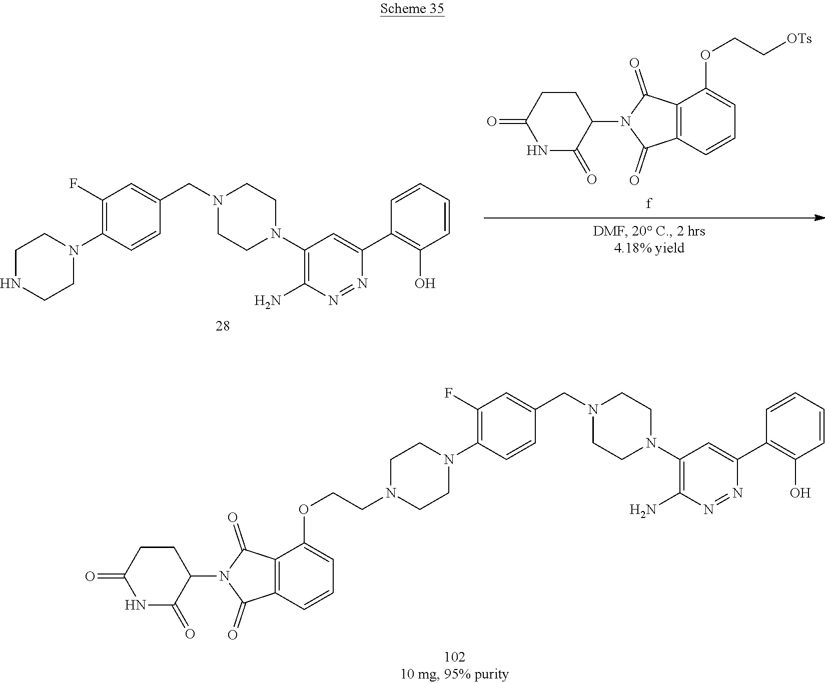 [SMILES: Cc1ccc(S(=O)(=O)OCCOc2cccc3c2C(=O)N(C2CCC(=O)NC2=O)C3=O)cc1 ; Nc1nnc(-c2ccccc2O)cc1N1CCN(Cc2ccc(N3CCNCC3)c(F)c2)CC1 ; Nc1nnc(-c2ccccc2O)cc1N1CCN(Cc2ccc(N3CCN(CCOc4cccc5c4C(=O)N(C4CCC(=O)NC4=O)C5=O)CC3)c(F)c2)CC1]

To a solution of compound 28 (203 mg, 431 umol) in DMF (2.00 mL) was added compound f (200 mg, 431 umol) (Scheme 35), Et₃N (43.6 mg, 431 umol, 60.0 uL). The mixture was stirred at 20° C. for 2 hrs. The mixtue was purified by Prep-HPLC (neutral condition) to give Compound 102 (15.0 mg, 18.0 umol, 4.18% yield, 91.8% purity) as a white solid.

LCMS: Rₜ=0.702 min, m/z=764.3 (M+H)⁺

HPLC: Rₜ=1.160 min, purity=95.9%

¹H NMR: (400 MHz, CDCl₃)

δ 14.1-13.4 (m, 1H), 8.42-8.39 (m, 1H), 7.88-7.76 (m, 1H), 7.58-7.46 (m, 1H), 7.48 (m, 1H), 7.32-7.30 (m, 1H), 7.28-7.27 (m, 1H), 7.25-7.22 (m, 1H), 7.18-7.08 (m, 3H), 7.06-6.89 (m, 2H), 4.98-4.94 (m, 1H), 4.82 (s, 2H), 4.42 (s, 2H), 3.54 (s, 2H), 3.25-3.18 (m, 8H), 3.06-3.01 (m, 2H), 2.94-2.76 (m, 7H), 2.73-2.55 (m, 4H), 2.17-2.08 (m, 1H)

F NMR: (400 MHz, CDCl₃)

SFC: Rt=0.555 min

### Example 8—Preparation of Compound 103

### General Procedure for Preparation of Compound 30

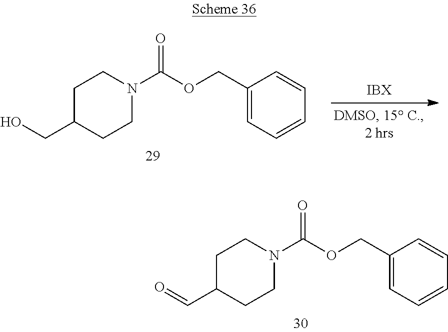 [SMILES: O=C(OCc1ccccc1)N1CCC(CO)CC1 ; O=CC1CCN(C(=O)OCc2ccccc2)CC1]

To a solution of compound 29 (4.00 g, 16.0 mmol, 1.00 eq) in DMSO (40.0 mL) was added IBX (6.74 g, 24.1 mmol, 1.50 eq) (Scheme 36). The mixture was stirred at 15° C. for 2 hrs. LCMS showed compound 29 was consumed completely and one main peak with desired mass was detected. The mixture was diluted with H₂O (100 mL) and extracted with EtOAc (100 mL×2). The combined organic layers were washed with brine (100 mL), dried over Na₂SO₄, filtered and concentrated under reduced pressure to give a residue to afford the crude compound 30 (3.70 g, crude) as a colorless oil and was used into the next step without further purification.

LCMS: Rₜ=0.993 min, m/z=464.2 (M+1)

¹H NMR: (400 MHz, CDCl₃)

δ 9.67 (s, H), 7.39˜7.31 (m, 5H), 5.14 (s, 2H), 4.08˜4.05 (m, 2H), 3.07˜3.01 (m, 2H), 2.48˜2.41 (m, 1H), 1.91 (m, 2H), 1.64˜1.54 (m, 2H)

### General Procedure for Preparation of Compound 31

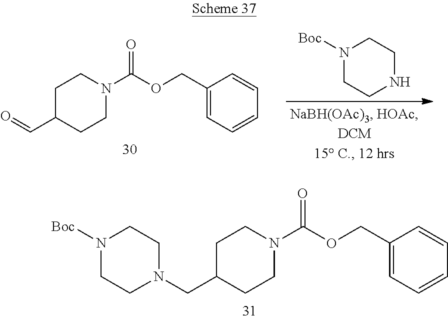 [SMILES: O=CC1CCN(C(=O)OCc2ccccc2)CC1 ; CC(C)(C)OC(=O)N1CCNCC1 ; CC(C)(C)OC(=O)N1CCN(CC2CCN(C(=O)OCc3ccccc3)CC2)CC1]

To a solution of compound 30 (3.70 g, 14.9 mmol, 1.00 eq) and tert-butyl piperazine-1-carboxylate (2.93 g, 15.7 mmol, 1.05 eq) in (40.0 mL) was added HOAc (898 mg, 14.9 mmol, 856 uL, 1.00 eq) and NaBH(OAc)₃ (4.76 g, 22.4 mmol, 1.50 eq) (Scheme 37). The mixture was stirred at 15° C. for 12 hrs. LCMS showed compound 30 was consumed completely and one main peak with desired mass was detected. The mixture was diluted with H₂O (100 mL) and extracted with DCM (100 mL×2). The combined organic layers were washed with brine (100 mL), dried over Na₂SO₄, filtered and concentrated under reduced pressure to give a residue to afford compound 31 (6.00 g, crude) as a white solid.

LCMS: Rₜ=0.798 min, m/z=418.2 (M+1)

¹H NMR: (400 MHz, CDCl₃)

δ 7.39˜7.30 (m, 5H), 5.13 (s, 2H), 4.18 (s, 2H), 3.41 (d, J=4.8 Hz, 4H), 2.77 (m, 2H), 2.34 (d, J=4.8 Hz, 4H), 2.17 (t, J=7.2 Hz, 2H), 1.77˜1.74 (m, 2H), 1.46 (s, 9H), 1.27 (m, 1H), 1.17˜1.07 (m, 2H)

### General Procedure for Preparation of Compound 32

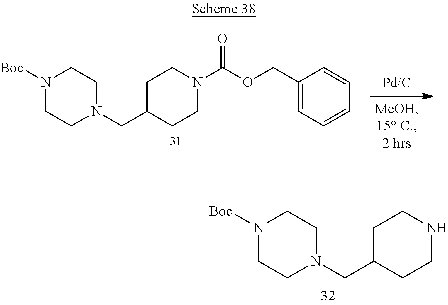 [SMILES: CC(C)(C)OC(=O)N1CCN(CC2CCN(C(=O)OCc3ccccc3)CC2)CC1 ; CC(C)(C)OC(=O)N1CCN(CC2CCNCC2)CC1]

To a solution of compound 31 (4.00 g, 9.58 mmol, 1.00 eq) in MeOH (40.0 mL) was added Pd/C (0.40 g, 958 umol, 10.0% purity, 0.10 eq) under N₂ (Scheme 38). The suspension was degassed under vacuum and purged with H₂ several times. The mixture was stirred under H₂ (15 psi) at 15° C. for 2 hrs. TLC (petroleum ether:ethyl acetate=1:1) showed Reactant 1 was consumed completely and one major new spot with larger polarity was detected. The mixture was filtered and the filtered cake washed with MeOH (100 ml), concentrated under reduced pressure to afford compound 32 (2.20 g, crude) as a gray solid.

¹H NMR: (400 MHz, CDCl₃)

δ 3.41 (d, J=4.8 Hz, 4H), 3.10˜3.07 (m, 2H), 2.63˜2.51 (m, 2H), 2.33 (d, J=4.8 Hz, 4H), 2.16 (t, J=7.2 Hz, 2H), 2.05 (s, 1H), 1.75˜1.72 (m, 2H), 1.65˜1.58 (m, 1H), 1.46 (s, 9H), 1.15˜1.05 (m, 2H)

### General Procedure for Preparation of Compound 33

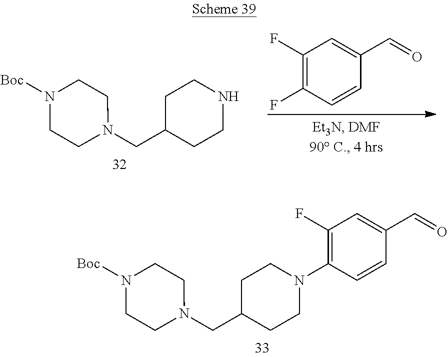 [SMILES: CC(C)(C)OC(=O)N1CCN(CC2CCNCC2)CC1 ; O=Cc1ccc(F)c(F)c1 ; CC(C)(C)OC(=O)N1CCN(CC2CCN(c3ccc(C=O)cc3F)CC2)CC1]

To a solution of compound 32 (2.20 g, 7.76 mmol, 1.00 eq) and 3,4-difluorobenzaldehyde (1.16 g, 8.15 mmol, 884 μL, 1.05 eq) in DMF (20.0 mL) was added Et₃N (1.18 g, 11.6 mmol, 1.62 mL, 1.50 eq) (Scheme 39). The mixture was stirred at 90° C. for 4 hrs. LCMS showed compound 32 was consumed completely and one main peak with desired mass was detected. The mixture was diluted with H₂O (100 mL) and extracted with EtOAc (50 mL×2). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=10/1 to 1/1, R_{f}=0.40) to afford compound 33 (2.30 g, 4.82 mmol, 62.0% yield, 84.9% purity) as a yellow solid.

LCMS: Rₜ=0.799 min, m/z=406.2 (M+1)

HPLC: Rₜ=1.535 min

¹H NMR: (400 MHz, CDCl₃)

δ 9.81˜9.80 (m, 1H), 7.56˜7.48 (m, 2H), 6.99˜6.96 (m, 1H), 3.70˜3.67 (m, 2H), 3.43 (m, 4H), 2.85˜2.79 (m, 2H), 2.36 (m, 4H), 2.23 (t, J=7.2 Hz, 2H), 1.88 (t, J=12.4 Hz, 2H), 1.74˜1.68 (m, 1H), 1.47 (s, 9H), 1.40˜1.36 (m, 2H)

### General Procedure for Preparation of Compound 34

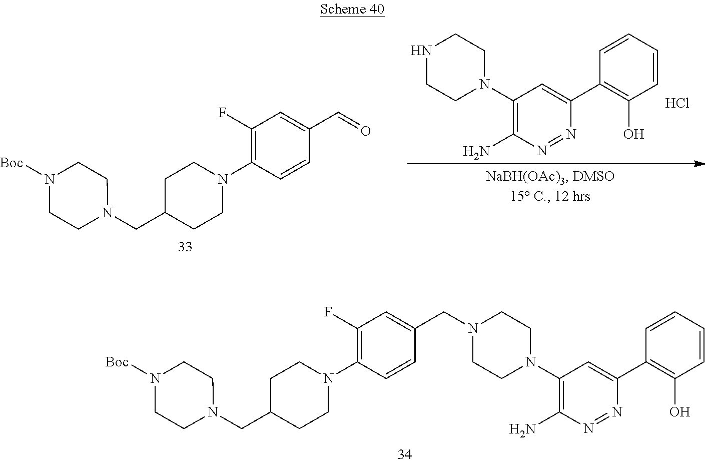 [SMILES: CC(C)(C)OC(=O)N1CCN(CC2CCN(c3ccc(C=O)cc3F)CC2)CC1 ; Nc1nnc(-c2ccccc2O)cc1N1CCNCC1 ; Cl ; CC(C)(C)OC(=O)N1CCN(CC2CCN(c3ccc(CN4CCN(c5cc(-c6ccccc6O)nnc5N)CC4)cc3F)CC2)CC1]

To a solution of compound 33 (1.00 g, 2.47 mmol, 1.00 eq) and 2-(6-amino-5-(piperazin-1-yl)pyridazin-3-yl)phenol hydrochloride (704 mg, 2.59 mmol, 1.05 eq) in DMSO (10.0 mL) was added NaBH(OAc)₃ (1.05 g, 4.94 mmol, 2.00 eq) (Scheme 40). The mixture was stirred at 15° C. for 12 hrs. LCMS showed compound 33 was consumed completely and one main peak with desired mass was detected. The mixture was diluted with H₂O (30 mL) and extracted with EtOAc (50 mL×2). The combined organic layers were washed with brine (30 mL×2), dried over Na₂SO₄, filtered and concentrated under reduced pressure to give a residue. The crude product was titurated with PE (10.0 mL) for 15 mins. Then the mixture was filtered and the filtered cake was washed with Petroleum ether (3 mL×2) to afford compound 34 (1.00 g, 1.37 mmol, 55.3% yield, 90.3% purity) as a yellow solid.

LCMS: Rₜ=0.741 min, m/z=661.3 (M+1)

¹H NMR: (400 MHz, CDCl₃)

δ 13.7 (s, 1H), 7.59˜7.57 (m, 1H), 7.32˜7.28 (m, 2H), 7.06˜7.00 (m, 3H), 6.94˜6.89 (m, 2H), 4.85 (s, 2H), 3.54 (s, 2H), 3.45 (m, 6H), 3.17 (s, 3H), 2.69˜2.64 (m, 6H), 2.40 (s, 4H), 2.28˜2.26 (m, 2H), 1.89˜1.87 (m, 2H), 1.66 (m, 1H), 1.47 (s, 9H), 1.40˜1.36 (m, 2H)

### General Procedure for Preparation of Compound 35

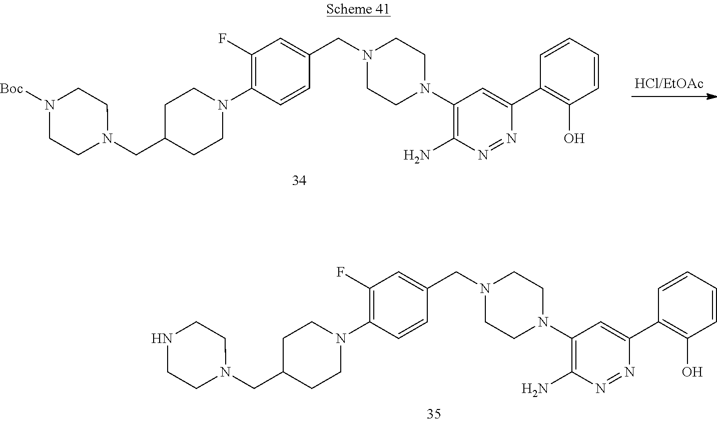 [SMILES: CC(C)(C)OC(=O)N1CCN(CC2CCN(c3ccc(CN4CCN(c5cc(-c6ccccc6O)nnc5N)CC4)cc3F)CC2)CC1 ; Nc1nnc(-c2ccccc2O)cc1N1CCN(Cc2ccc(N3CCC(CN4CCNCC4)CC3)c(F)c2)CC1]

To a solution of compound 34 (500 mg, 756 umol, 1.00 eq) in HCl/EtOAc (5.00 mL) was stirred at 15° C. for 2 hrs (Scheme 41). LCMS showed compound 34 was consumed completely and one main peak with desired mass was detected. The mixture was concentrated under reduced pressure to give a residue to afford compound 35 (400 mg, crude, HCl) as yellow solid.

LCMS: Rₜ=0.711 min, m/z=561.3 (M+1)

¹H NMR: (400 MHz, CDCl₃)

δ 12.7 (s, 1H), 10.2 (s, 2H), 7.59˜7.57 (m, 1H), 7.32˜7.28 (m, 2H), 7.06˜7.00 (m, 3H), 6.94˜6.89 (m, 2H), 4.85 (s, 2H), 3.54 (s, 2H), 3.45 (m, 6H), 3.17 (s, 3H), 2.69˜2.64 (m, 6H), 2.40 (s, 4H), 2.28˜2.26 (m, 2H), 1.89˜1.87 (m, 2H), 1.66 (m, 1H), 1.40˜1.36 (m, 2H)

### General Procedure for Preparation of Compound 103

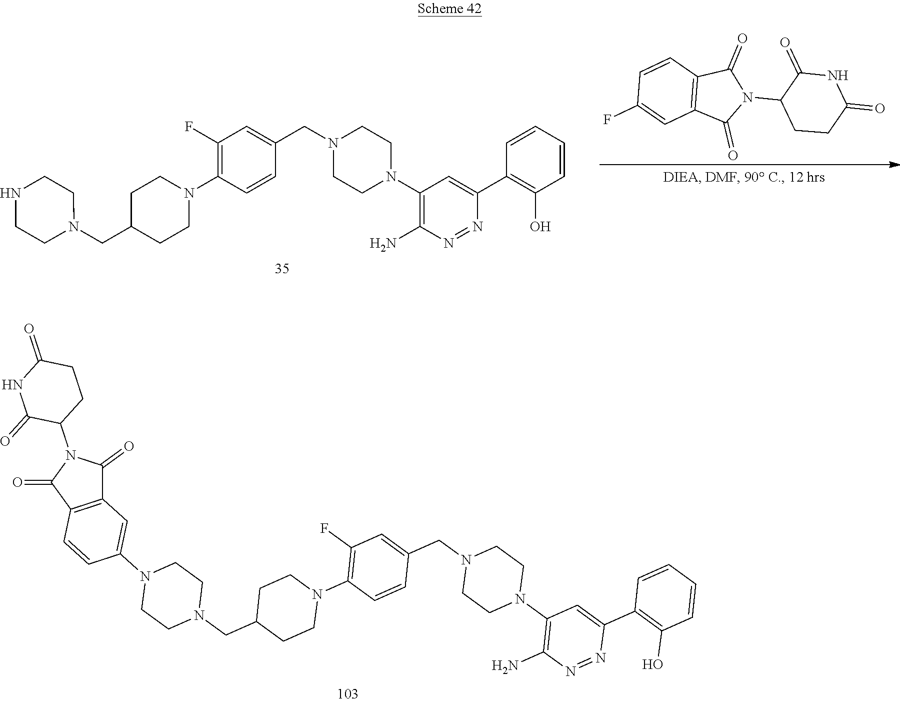 [SMILES: Nc1nnc(-c2ccccc2O)cc1N1CCN(Cc2ccc(N3CCC(CN4CCNCC4)CC3)c(F)c2)CC1 ; O=C1CCC(N2C(=O)c3ccc(F)cc3C2=O)C(=O)N1 ; Nc1nnc(-c2ccccc2O)cc1N1CCN(Cc2ccc(N3CCC(CN4CCN(c5ccc6c(c5)C(=O)N(C5CCC(=O)NC5=O)C6=O)CC4)CC3)c(F)c2)CC1]

To a solution of compound 35 (200 mg, 335 umol, 1.00 eq, HCl) and 2-(2,6-dioxopiperidin-3-yl)-5-fluoroisoindoline-1,3-dione (97.1 mg, 352 umol, 1.05 eq) in DMF (2.00 mL) was added DIEA (216 mg, 1.67 mmol, 292 μL, 5.00 eq) (Scheme 42). The mixture was stirred at 90° C. for 12 hrs. LCMS showed compound 35 was consumed completely and one main peak with desired mass was detected. The mixture was diluted with H₂O (20.0 mL) and extracted with ethyl acetate (20.0 mL×2). The combined organic layers were washed with brine (10.0 mL×2), dried over Na₂SO₄, filtered and concentrated under reduced pressure to give a residue. The residue was purified by Prep-HPLC (column: Waters Xbridge C18 150*50 mm*10 um; mobile phase: [water(10 mM NH4HCO3)-ACN]; B %: 48%-78%, 11 min) to afford Compound 103 (50.0 mg, 59.5 umol, 17.8% yield, 97.3% purity) as a yellow solid.

LCMS: Rₜ=1.061 min, m/z=817.0 (M+1)

HPLC: Rₜ=2.611 min

¹H NMR (400 MHz, CDCl₃)

FNMR:

δ 13.7 (s, 1H), 8.20 (s, 1H), 7.70 (t, J=8.4 Hz, 2H), 7.60˜7.58 (m, 1H), 7.33 (s, 2H), 7.31˜7.29 (m, 2H), 4.97˜4.93 (m, 1H), 4.82 (s, 2H), 3.55 (s, 2H), 3.46˜3.40 (m, 6H), 3.18 (s, 4H), 2.92˜2.77 (m, 3H), 2.74˜2.72 (m, 1H), 2.71˜2.66 (m, 5H), 2.61 (s, 4H), 2.33 (t, J=6.8 Hz, 2H), 2.18˜2.09 (m, 1H), 1.89 (t, J=12.4 Hz, 2H), 1.53˜1.40 (m, 3H)

Table 1 identifies compounds disclosed in Examples 1-8.

TABLE 1
Compounds 1-35, a_1, a-e, 1A-6A, and COMPOUNDS 101-108

| Cpd # | Structure |
|---|---|
| 1 | 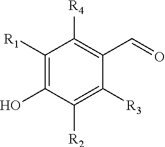 |
| 2 | 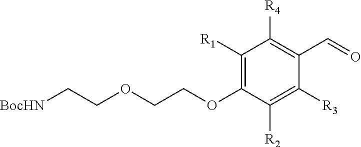 |
| 3 | 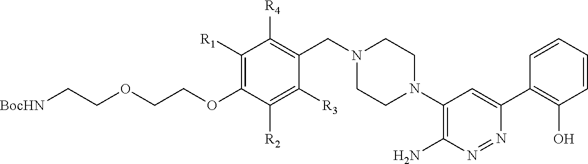 |
| 4 | 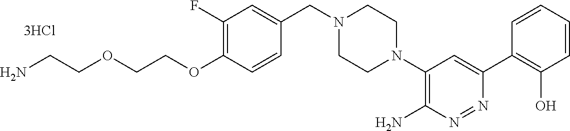 [SMILES: NCCOCCOc1ccc(CN2CCN(c3cc(-c4ccccc4O)nnc3N)CC2)cc1F] |
| 5 | 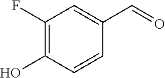 |
| 6 | 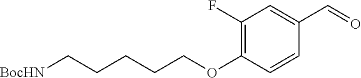 [SMILES: CC(C)(C)OC(=O)NCCCCCOc1ccc(C=O)cc1F] |
| 7 | 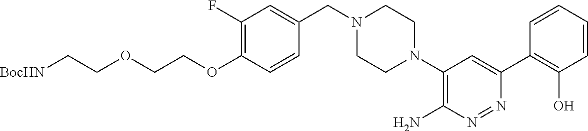 [SMILES: CC(C)(C)OC(=O)NCCOCCOc1ccc(CN2CCN(c3cc(-c4ccccc4O)nnc3N)CC2)cc1F] |
| 8 | 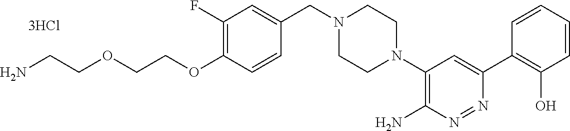 [SMILES: NCCOCCOc1ccc(CN2CCN(c3cc(-c4ccccc4O)nnc3N)CC2)cc1F] |
| 9 | 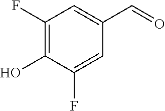 [SMILES: O=Cc1cc(F)c(O)c(F)c1] |
| 10 | 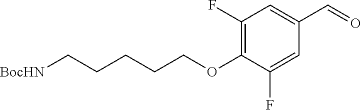 [SMILES: CC(C)(C)OC(=O)NCCCCCOc1c(F)cc(C=O)cc1F] |
| 11 | 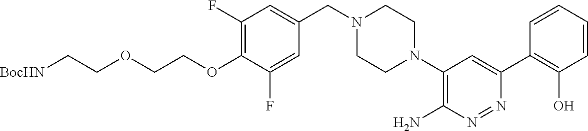 [SMILES: CC(C)(C)OC(=O)NCCOCCOc1c(F)cc(CN2CCN(c3cc(-c4ccccc4O)nnc3N)CC2)cc1F] |
| 12 | 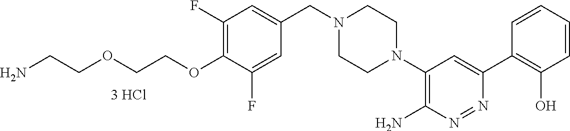 [SMILES: NCCOCCOc1c(F)cc(CN2CCN(c3cc(-c4ccccc4O)nnc3N)CC2)cc1F] |
| 13 | 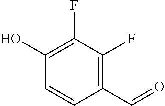 [SMILES: O=Cc1ccc(O)c(F)c1F] |
| 14 | 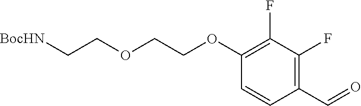 [SMILES: CC(C)(C)OC(=O)NCCOCCOc1ccc(C=O)c(F)c1F] |
| 15 | 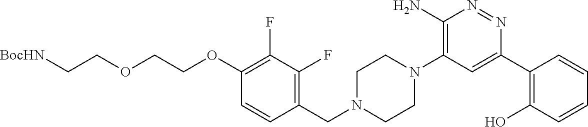 [SMILES: CC(C)(C)OC(=O)NCCOCCOc1ccc(CN2CCN(c3cc(-c4ccccc4O)nnc3N)CC2)c(F)c1F] |
| 16 | 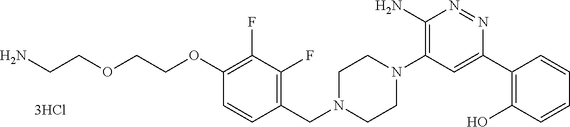 [SMILES: NCCOCCOc1ccc(CN2CCN(c3cc(-c4ccccc4O)nnc3N)CC2)c(F)c1F] |
| 17 | 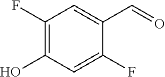 [SMILES: O=Cc1cc(F)c(O)cc1F] |
| 18 | 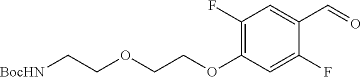 [SMILES: CC(C)(C)OC(=O)NCCOCCOc1cc(F)c(C=O)cc1F] |
| 19 | 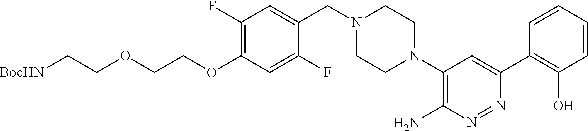 [SMILES: CC(C)(C)OC(=O)NCCOCCOc1cc(F)c(CN2CCN(c3cc(-c4ccccc4O)nnc3N)CC2)cc1F] |
| 20 | 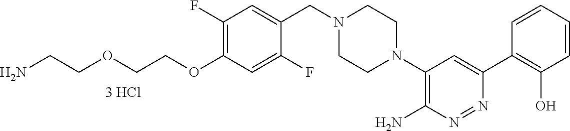 [SMILES: NCCOCCOc1cc(F)c(CN2CCN(c3cc(-c4ccccc4O)nnc3N)CC2)cc1F] |
| 21 | 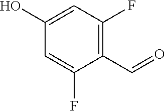 [SMILES: O=Cc1c(F)cc(O)cc1F] |
| 22 | 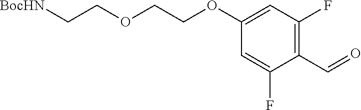 [SMILES: CC(C)(C)OC(=O)NCCOCCOc1cc(F)c(C=O)c(F)c1] |
| 23 | 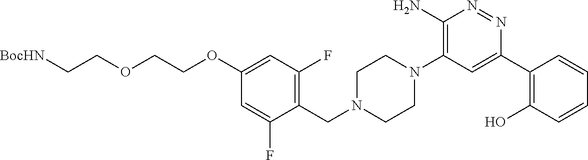 [SMILES: CC(C)(C)OC(=O)NCCOCCOc1cc(F)c(CN2CCN(c3cc(-c4ccccc4O)nnc3N)CC2)c(F)c1] |
| 24 | 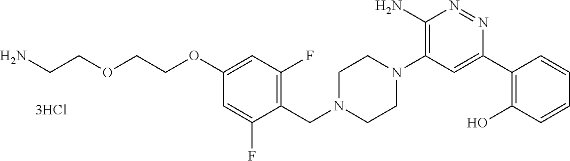 [SMILES: NCCOCCOc1cc(F)c(CN2CCN(c3cc(-c4ccccc4O)nnc3N)CC2)c(F)c1] |
| 25 | 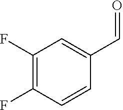 |
| 26 | 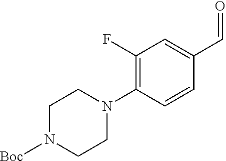 [SMILES: CC(C)(C)OC(=O)N1CCN(c2ccc(C=O)cc2F)CC1] |
| 27 | 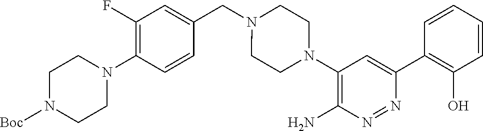 [SMILES: CC(C)(C)OC(=O)N1CCN(c2ccc(CN3CCN(c4cc(-c5ccccc5O)nnc4N)CC3)cc2F)CC1] |
| 28 | 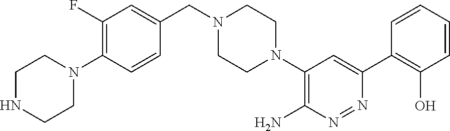 [SMILES: Nc1nnc(-c2ccccc2O)cc1N1CCN(Cc2ccc(N3CCNCC3)c(F)c2)CC1] |
| 29 | 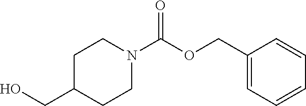 [SMILES: O=C(OCc1ccccc1)N1CCC(CO)CC1] |
| 30 | 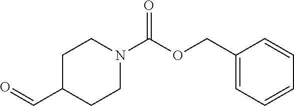 [SMILES: O=CC1CCN(C(=O)OCc2ccccc2)CC1] |
| 31 | 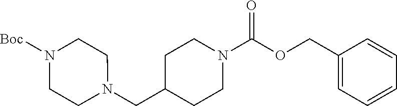 [SMILES: CC(C)(C)OC(=O)N1CCN(CC2CCN(C(=O)OCc3ccccc3)CC2)CC1] |
| 32 | 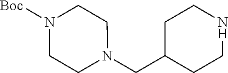 [SMILES: CC(C)(C)OC(=O)N1CCN(CC2CCNCC2)CC1] |
| 33 | 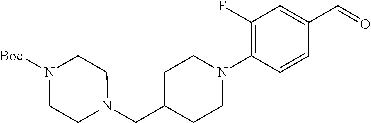 [SMILES: CC(C)(C)OC(=O)N1CCN(CC2CCN(c3ccc(C=O)cc3F)CC2)CC1] |
| 34 | 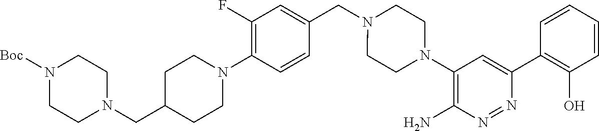 [SMILES: CC(C)(C)OC(=O)N1CCN(CC2CCN(c3ccc(CN4CCN(c5cc(-c6ccccc6O)nnc5N)CC4)cc3F)CC2)CC1] |
| 35 | 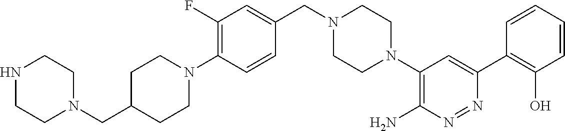 [SMILES: Nc1nnc(-c2ccccc2O)cc1N1CCN(Cc2ccc(N3CCC(CN4CCNCC4)CC3)c(F)c2)CC1] |
| a_1 | 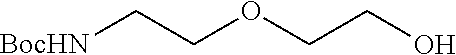 [SMILES: CC(C)(C)OC(=O)NCCOCCO] |
| a | 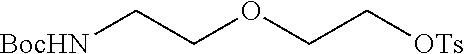 [SMILES: Cc1ccc(S(=O)(=O)OCCOCCNC(=O)OC(C)(C)C)cc1] |
| b | 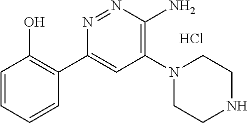 [SMILES: Nc1nnc(-c2ccccc2O)cc1N1CCNCC1 ; Cl] |
| c | 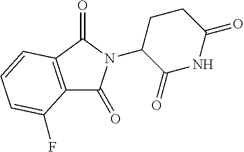 [SMILES: O=C1CCC(N2C(=O)c3cccc(F)c3C2=O)C(=O)N1] |
| d | 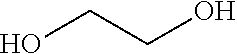 |
| e | 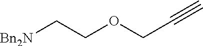 |
| f | 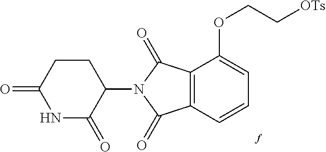 [SMILES: Cc1ccc(S(=O)(=O)OCCOc2cccc3c2C(=O)N(C2CCC(=O)NC2=O)C3=O)cc1] |
| 1A | 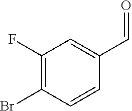 |
| 2A | 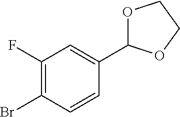 [SMILES: Fc1cc(C2OCCO2)ccc1Br] |
| 3A | 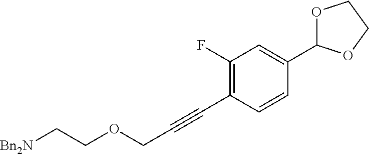 |
| 4A | 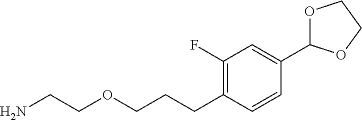 [SMILES: NCCOCCCc1ccc(C2OCCO2)cc1F] |
| 5A | 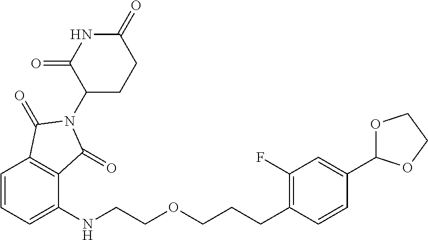 [SMILES: O=C1CCC(N2C(=O)c3cccc(NCCOCCCc4ccc(C5OCCO5)cc4F)c3C2=O)C(=O)N1] |
| 6A | 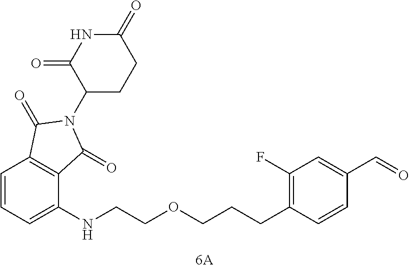 [SMILES: O=Cc1ccc(CCCOCCNc2cccc3c2C(=O)N(C2CCC(=O)NC2=O)C3=O)c(F)c1] |
| COMPOUND 101 | 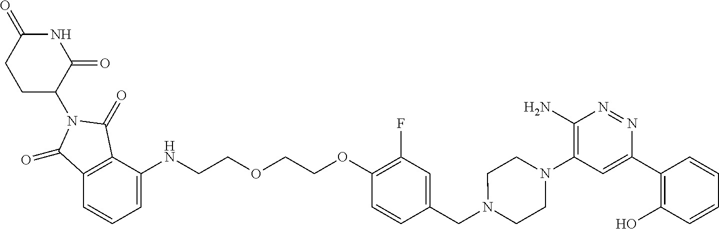 [SMILES: Nc1nnc(-c2ccccc2O)cc1N1CCN(Cc2ccc(OCCOCCNc3cccc4c3C(=O)N(C3CCC(=O)NC3=O)C4=O)c(F)c2)CC1] |
| COMPOUND 102 | 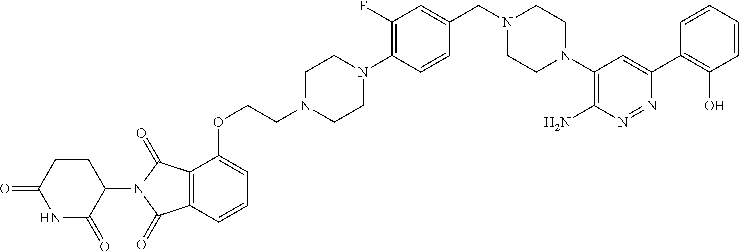 [SMILES: Nc1nnc(-c2ccccc2O)cc1N1CCN(Cc2ccc(N3CCN(CCOc4cccc5c4C(=O)N(C4CCC(=O)NC4=O)C5=O)CC3)c(F)c2)CC1] |
| COMPOUND 103 | 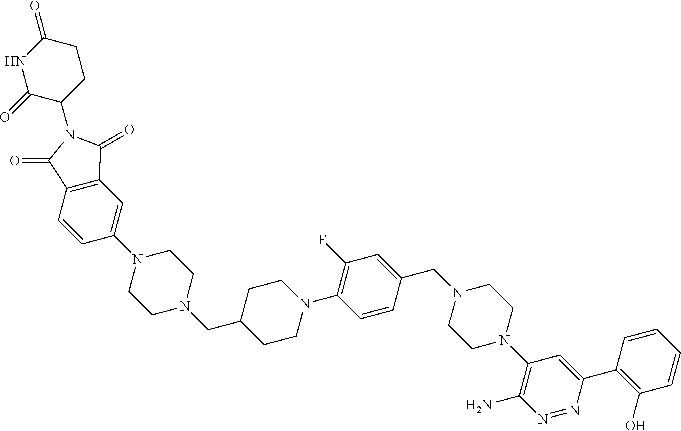 [SMILES: Nc1nnc(-c2ccccc2O)cc1N1CCN(Cc2ccc(N3CCC(CN4CCN(c5ccc6c(c5)C(=O)N(C5CCC(=O)NC5=O)C6=O)CC4)CC3)c(F)c2)CC1] |
| COMPOUND 104 | 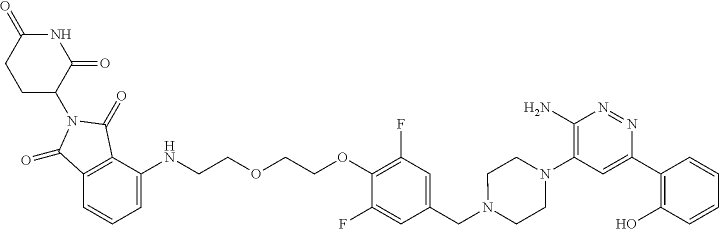 [SMILES: Nc1nnc(-c2ccccc2O)cc1N1CCN(Cc2cc(F)c(OCCOCCNc3cccc4c3C(=O)N(C3CCC(=O)NC3=O)C4=O)c(F)c2)CC1] |
| COMPOUND 105 | 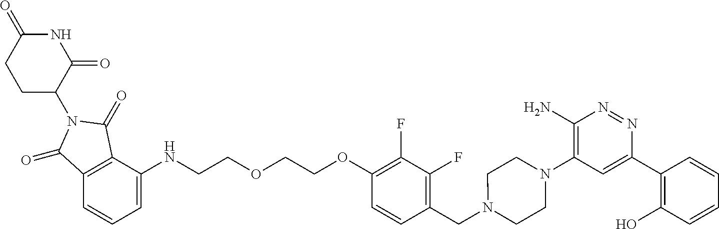 [SMILES: Nc1nnc(-c2ccccc2O)cc1N1CCN(Cc2ccc(OCCOCCNc3cccc4c3C(=O)N(C3CCC(=O)NC3=O)C4=O)c(F)c2F)CC1] |
| COMPOUND 106 | 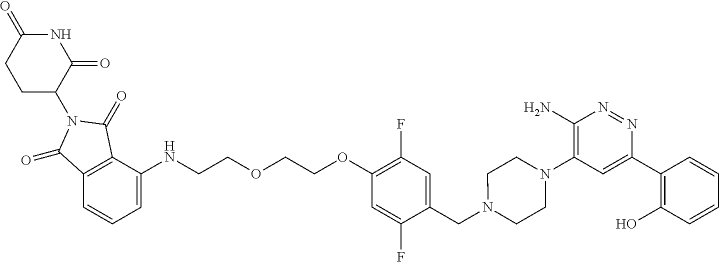 [SMILES: Nc1nnc(-c2ccccc2O)cc1N1CCN(Cc2cc(F)c(OCCOCCNc3cccc4c3C(=O)N(C3CCC(=O)NC3=O)C4=O)cc2F)CC1] |
| COMPOUND 107 | 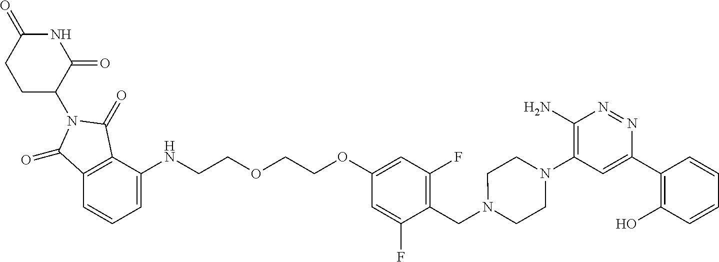 [SMILES: Nc1nnc(-c2ccccc2O)cc1N1CCN(Cc2c(F)cc(OCCOCCNc3cccc4c3C(=O)N(C3CCC(=O)NC3=O)C4=O)cc2F)CC1] |
| COMPOUND 108 | 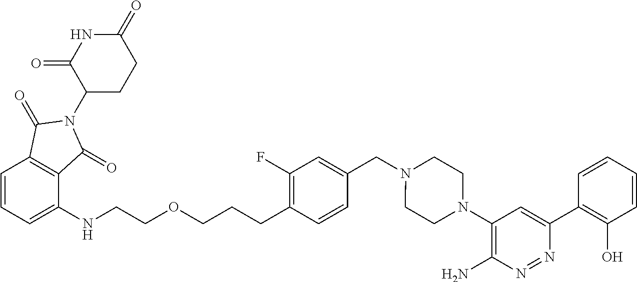 [SMILES: Nc1nnc(-c2ccccc2O)cc1N1CCN(Cc2ccc(CCCOCCNc3cccc4c3C(=O)N(C3CCC(=O)NC3=O)C4=O)c(F)c2)CC1] |

### Example 9—SMARCA2 Degradation in wild-type SMARCA4 and mutant SMARCA4 cell lines Using SMARCA2 PROTAC Compound 101

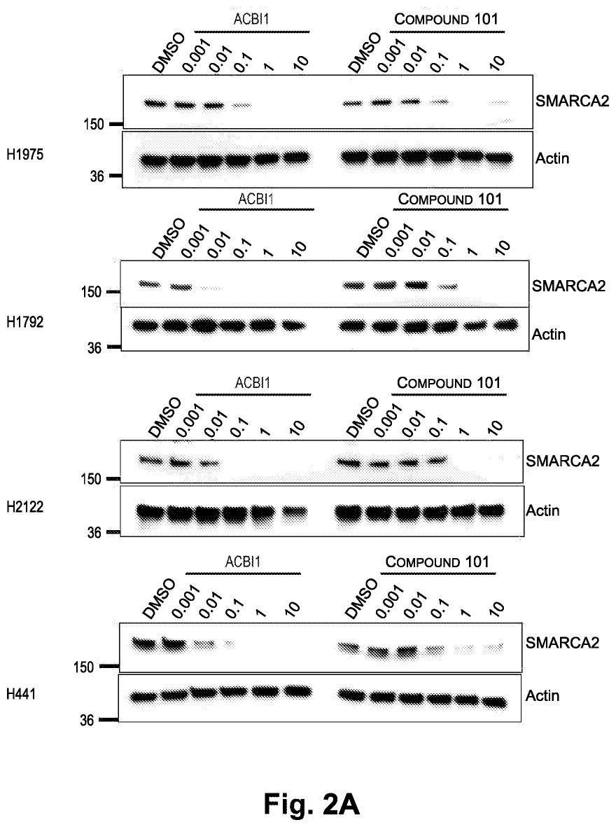

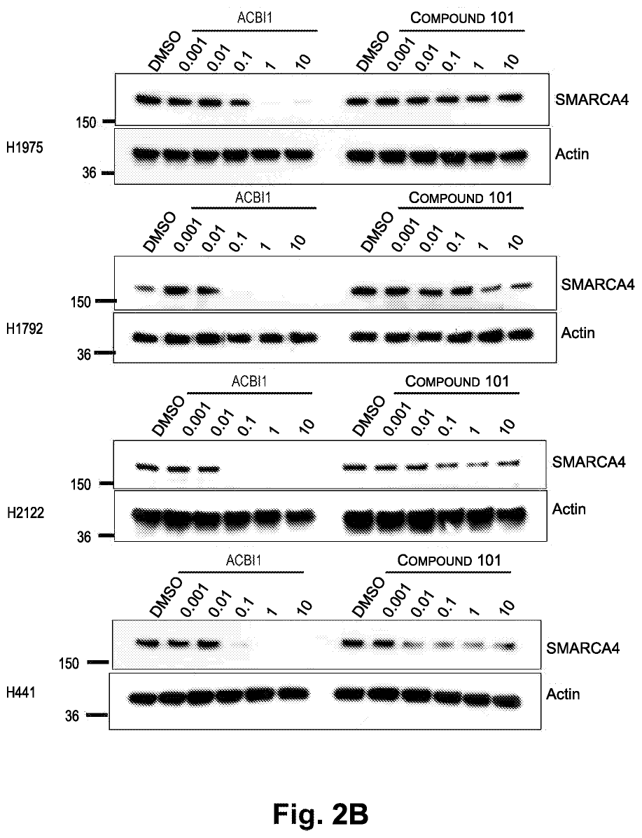

Cell lines with wild-type SMARCA4 (H1975, H1792, H2122, and H441) were treated with DMSO or 0.001, 0.01, 0.1, 1, or 10 μm of ACBI1 (degrader of SMARCA2, SMARCA4 and polybromo-associated BAF complex PBRM1) or Compound 101 and expression levels of SMARCA2 (FIG. 2A) and SMARCA4 (FIG. 2B) were detected by western blot. Both ACBI1 and Compound 101 degraded SMARCA2 and SMARCA4 when used in increasing concentrations. However, Compound 101 was greater than 100 times more selective in degrading SMARCA2 compared to SMARCA4 than ACBI1 (Tables 2 and 3 and FIGS. 2A and 2B). Table 2 shows the results for SMARCA2 and Table 3 shows the results for SMARCA4.

TABLE 2
SMARCA2 Degradation with COMPOUND 101 and ACBI1

| | | ACBI1 | | ACBI1 | COMPOUND |
|---|---|---|---|---|---|
| | Cell | Dmax | COMPOUND | DC₅₀ | 101 DC₅₀ |
| Type | line | (%) | 101 Dmax (%) | (μM) | (μM) |
| Wild-type | H1975 | 98 | 93 | 0.051 | 0.103 |
| Wild-type | H1792 | 97 | 89 | 0.007 | 0.094 |
| Wild-type | H2122 | 95 | 94 | 0.013 | 0.292 |
| Wild-type | H441 | 97 | 67 | 0.011 | 0.099 |

TABLE 3
SMARCA4 Degradation with COMPOUND 101 and ACBI1

| | | ACBI1 | | ACBI1 | COMPOUND |
|---|---|---|---|---|---|
| | Cell | Dmax | COMPOUND | DC₅₀ | 101 DC₅₀ |
| Type | line | (%) | 101 Dmax (%) | (μM) | (μM) |
| Wild-type | H1975 | 98 | N/A | 0.098 | >10 |
| Wild-type | H1792 | 98 | 38 | 0.018 | >10 |
| Wild-type | H2122 | 97 | 47 | 0.032 | >10 |
| Wild-type | H441 | 99 | 50 | 0.036 | >10 |

The data showed that both compounds degraded SMARCA2 at a Dₘₐₓ (maximum degradation observed as percentage of DMSO control) of between 67% and 98% (FIG. 2A, Table 2). However, only ACBI1 showed a Dₘₐₓ of 97-99% in SMARCA4 degradation, whereas the Dₘₐₓ for Compound 101 was either not measurable or degraded SMARCA4 at a Dₘₐₓ between 38% and 50% (FIG. 2B, Table 3), confirming the selectivity of Compound 101 for SMARCA2. Further, in wild-type SMARCA4 cells, Compound 101 had a DC₅₀ for SMARCA2 of about 99 nM to about 292 nm (Table 2) while its DC₅₀ for SMARCA4 was above 10 M (Table 3).

### Example 10—SMARCA2 PROTAC Compound 101 Specifically Reduces Cell Proliferation in SMARCA4 Mutant Cell Lines

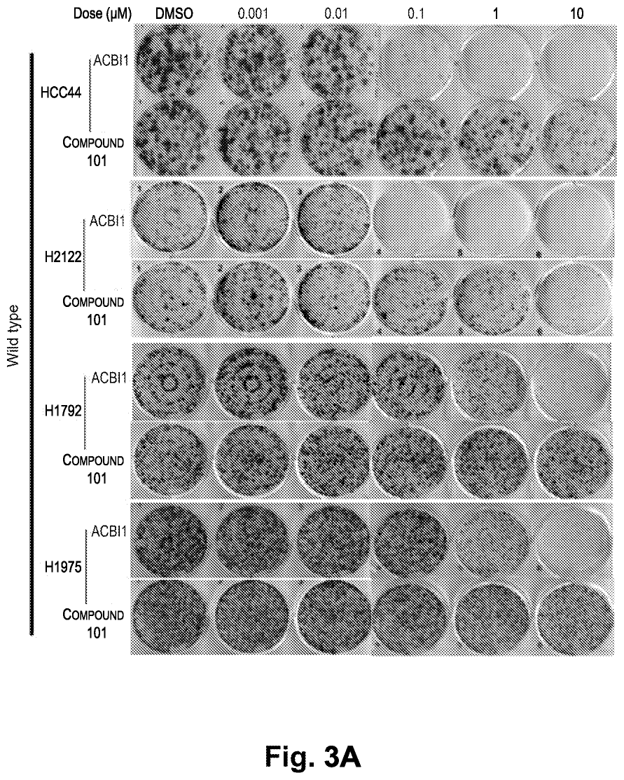

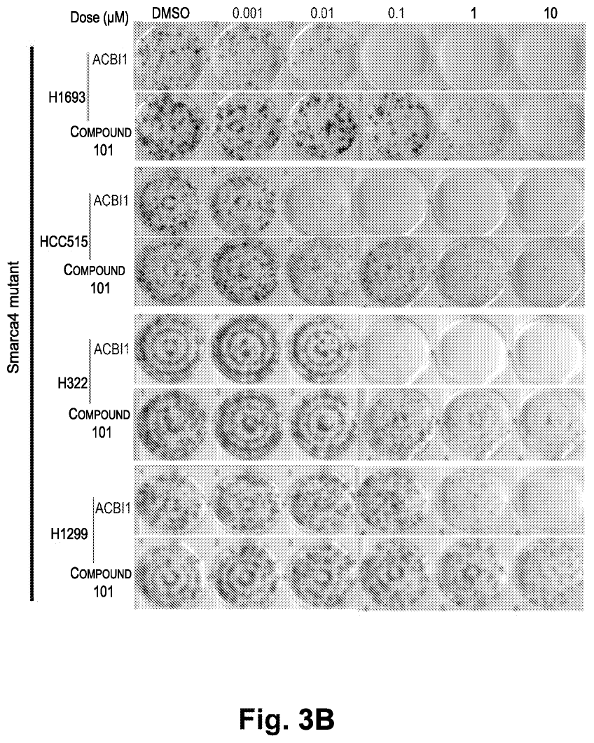

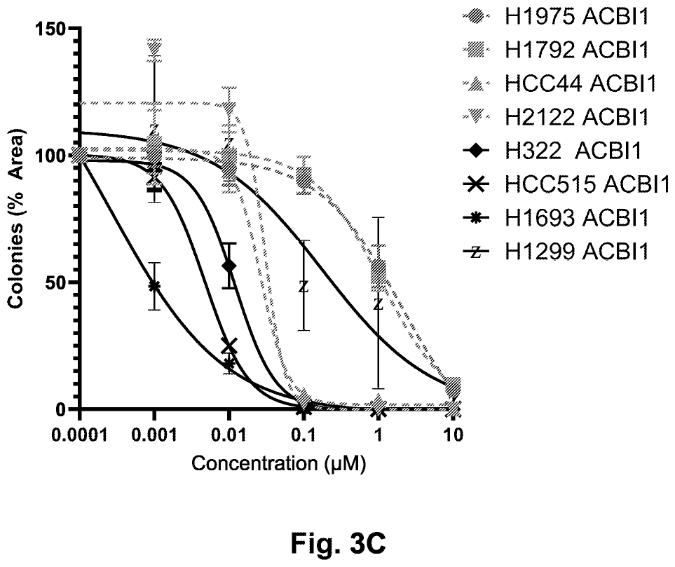

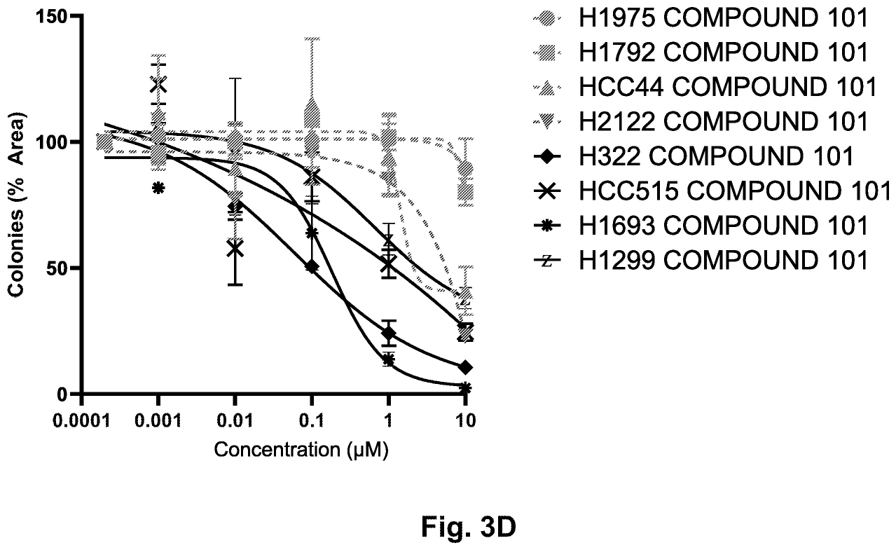

Cell lines with wild-type SMARCA4 (HCC44, H2122, H1792 and H1975) and cell lines with mutant SMARCA4 (H1693, HCC515, H322, and H1299) were seeded in appropriate dilutions to form colonies in about 1-3 weeks. Cells were treated with ACBI1 or Compound 101 at concentrations of 0.001, 0.01, 0.1, 1, and 10 μm. Colonies were fixed with glutaraldehyde (6% v/v), stained with crystal violet (0.5% w/v) and the percent area of colonies was determined (FIGS. 3A and 3B). In cell lines with wild-type SMARCA4, inhibition of colony growth was more pronounced after treatment with ACBI1 than Compound 101 (FIG. 3A). More pronounced colony growth inhibition was observed with both ACBI1 and Compound 101 in mutant SMARCA4 cell lines (FIG. 3B). While ACBI1 inhibited wild-type and mutant SMARCA4 cells (FIG. 3C), Compound 101 selectively inhibited SMARCA4 mutant cells (FIG. 3D).

Cell lines with wild-type SMARCA4 (HCC44, H2122, H1792. H1975, H441, H2228, HCC827, HCC2279, and HOP62) and mutant SMARCA4 (H1693, HCC515, H322, H1299, A549, HCC366, H2023, H2126, H1793, H838, and H2935) were further tested for SMARCA2 and SMARCA4 Dₘₐₓ, DC₅₀, and IC₅₀ (concentration at which 50% of cellular proliferation is inhibited).

Compound 101 degraded SMARCA2 with a Dₘₐₓ between 82% and 99% in wild-type SMARCA4 cells (Table 4) and between 81% and 99% in mutant SMARCA4 cells (Table 5). Further, the DC₅₀ for SMARCA2 degradation of Compound 101 was between 32 nM and 292 nM in wild-type SMARCA4 cells (Table 4) and between 8 nM and 113 nM in mutant SMARCA4 cells (Table 4).

Compound 101 degraded SMARCA4 with a Dₘₐₓ between 38% and 79% in wild-type SMARCA4 cells (Table 4) and the DC₅₀ for SMARCA4 degradation of Compound 101 was between 560 nM and above 10 M in these cells (Table 4).

The IC₅₀ for Compound 101 in wild-type SMARCA4 cells was between 0.97 M and 10 M (Table 4), whereas the IC₅₀ for Compound 101 in mutant SMARCA4 cells was between 0.93 nM and >10 M (Table 5). These data confirmed that Compound 101 selectively inhibited cell proliferation in mutant SMARCA4 cell lines.

TABLE 4
Cell lines with wild-type SMARCA4

| | | COMPOUND | COMPOUND | COMPOUND | |
|---|---|---|---|---|---|
| | COMPOUND | 101 | 101 | 101 | IC₅₀ |
| SMARCA4 | 101 SMARCA2 | SMARCA2 | SMARCA4 | SMARCA4 | COMPOUND |
| wt cell lines | Dₘₐₓ (%) | DC₅₀ (μM) | Dₘₐₓ (%) | DC₅₀ (μM) | 101 (μM) |
| H1975 | 93 | 0.103 | NA | >10 | >10 |
| H1792 | 94 | 0.094 | 38 | >10 | 8.7 |
| H2122 | 94 | 0.292 | 47 | >10 | 2.6 |
| H441 | 67 | 0.099 | 37 | >10 | >10 |
| H2228 | 82 | 0.106 | 25 | >10 | 0.97 |
| HCC827 | 83 | 0.07 | 39 | >10 | >10 |
| HCC2279 | 99 | 0.032 | 79 | 0.56 | >10 |
| HCC44 | 99 | 0.069 | 39 | >10 | 2.5 |
| HOP62 | 99 | 0.033 | 77 | 1.41 | Not determined |

TABLE 5
Cell lines with mutant SMARCA4

| | COMPOUND | COMPOUND | COMPOUND | COMPOUND | |
|---|---|---|---|---|---|
| SMARCA4 | 101 | 101 | 101 | 101 | IC₅₀ |
| mutant cell | SMARCA2 | SMARCA2 | SMARCA4 | SMARCA4 | COMPOUND |
| lines | Dₘₐₓ (%) | DC₅₀ (μM) | Dₘₐₓ (%) | DC₅₀ (μM) | 101 (μM) |
| H1693 | 84 | 0.113 | No expression | No expression | 0.141 |
| HCC515 | 81 | 0.083 | No expression | No expression | 0.817 |
| HCC366 | 99 | 0.013 | No expression | No expression | 0.242 |
| A549 | 96 | 0.057 | No expression | No expression | >10 |
| H322 | 99 | 0.027 | No expression | No expression | 0.093 |
| H2023 | 99 | 0.042 | No expression | No expression | 0.483 |
| H2126 | 99 | 0.01 | No expression | No expression | ~.02 |
| H1793 | 99 | 0.002 | No expression | No expression | 0.9 |
| H838 | 98 | 0.026 | No expression | No expression | Not determined |
| H2935 | 83 | 0.008 | No expression | No expression | Not determined |

### Example 11—SMARCA2 PROTAC Compound 103

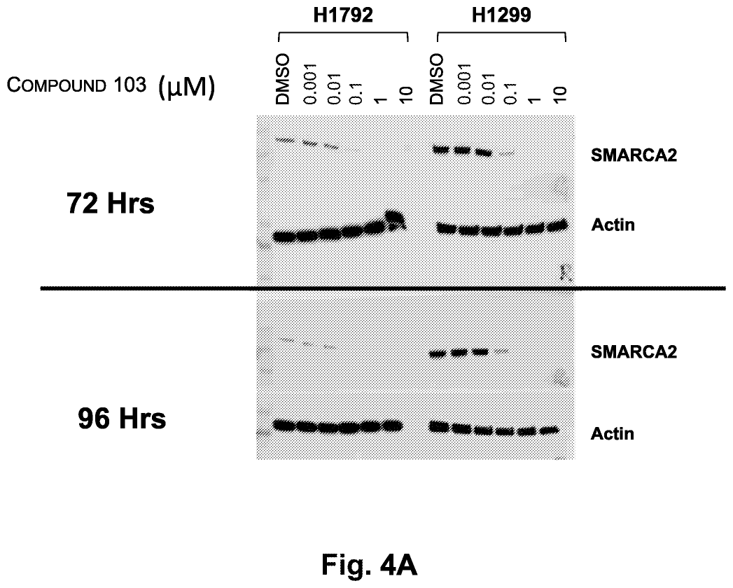

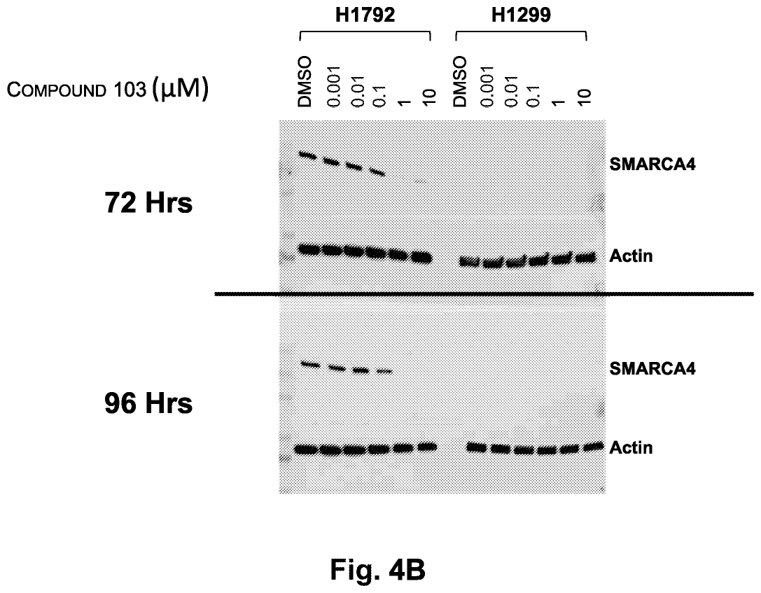

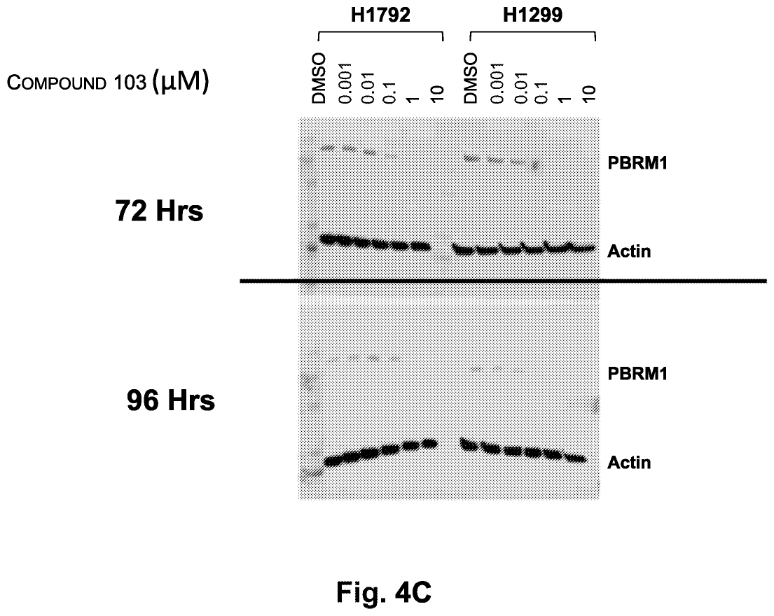

Cell lines with wild-type SMARCA4 (H1792) and mutant SMARCA4 (H1299) were treated for 72 h or 96 h with DMSO or 0.001, 0.01, 0.1, 1, or 10 μm Compound 103 and analyzed by western blot for SMARCA2, SMARCA4, and PBRM1 expression (FIGS. 4A, 4B, and 4C). Compound 103 efficiently degraded SMARCA2 (FIG. 4A) and reduced expression of SMARCA4 in H1792 cells (FIG. 4B) but did not have a significant effect on the expression of PBRM1 (FIG. 4C). Compared to Compound 101, Compound 103 significantly inhibited SMARCA2 expression in both H1792 and H1299 cells at 100 nM (FIG. 4A) at which concentration Compound 101 achieved an inhibition of maximal 50% in SMARCA4 mutant cells and less than about 10% in wild-type SMARCA4 cells (see FIG. 3D).

### Example 12—SMARCA2 PROTAC Compound 102

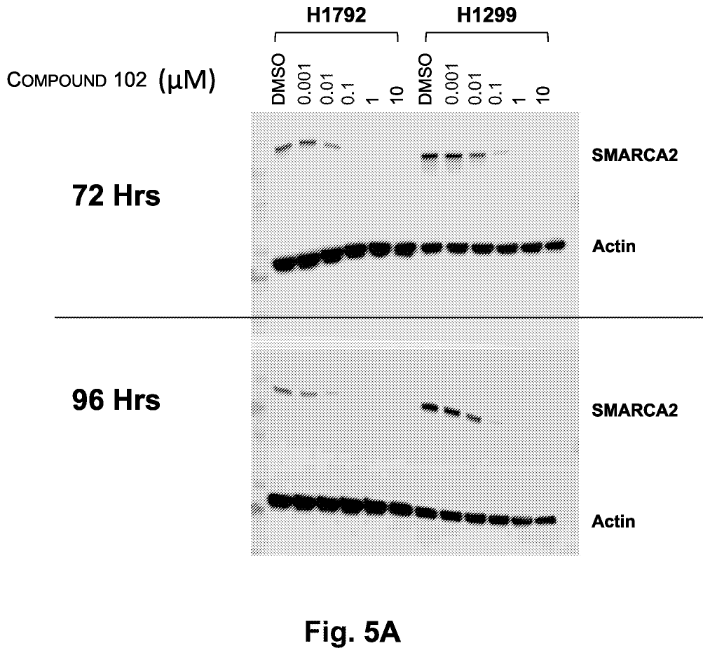

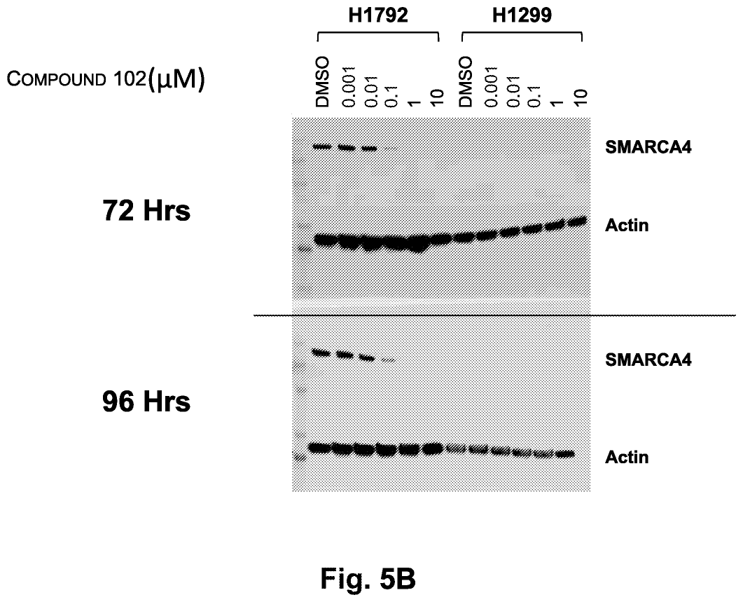

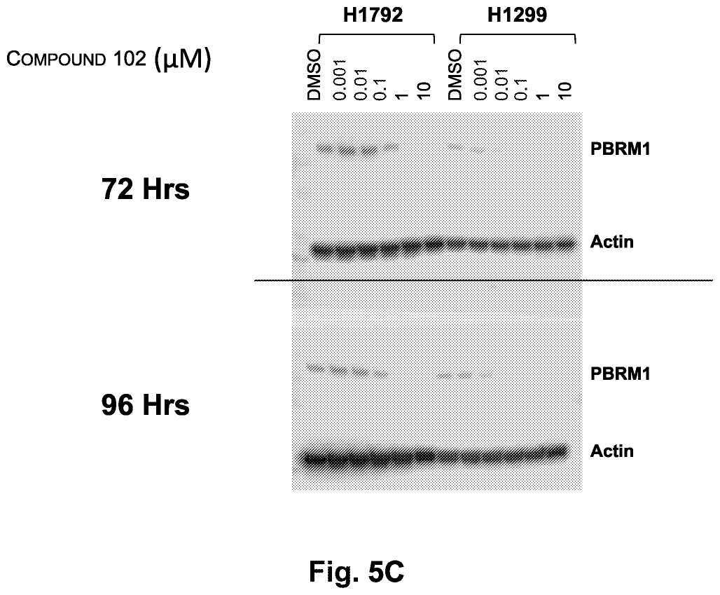

Cell lines with wild-type SMARCA4 (H1792) and mutant SMARCA4 (H1299) were treated for 72 h or 96 h with DMSO or 0.001, 0.01, 0.1, 1, or 10 μm Compound 102 and analyzed by western blot for SMARCA2, SMARCA4, and PBRM1 expression (FIGS. 5A, 5B, and 5C). Compound 102 efficiently degraded SMARCA2 (FIG. 5A) and reduced expression of SMARCA4 (FIG. 5B) but did not have a significant effect on the expression of PBRM1 (FIG. 5C). Compared to Compound 101, Compound 102 significantly inhibited SMARCA2 expression in both H1792 and H1299 cells at 100 nM (FIG. 5A) at which concentration Compound 101 achieved an inhibition of maximal 50% in SMARCA4 mutant cells and less than about 10% in wild-type SMARCA4 cells (see FIG. 3D).

### Example 13—SMARCA2 PROTAC Compounds 104, 105, 106, 107, and 108

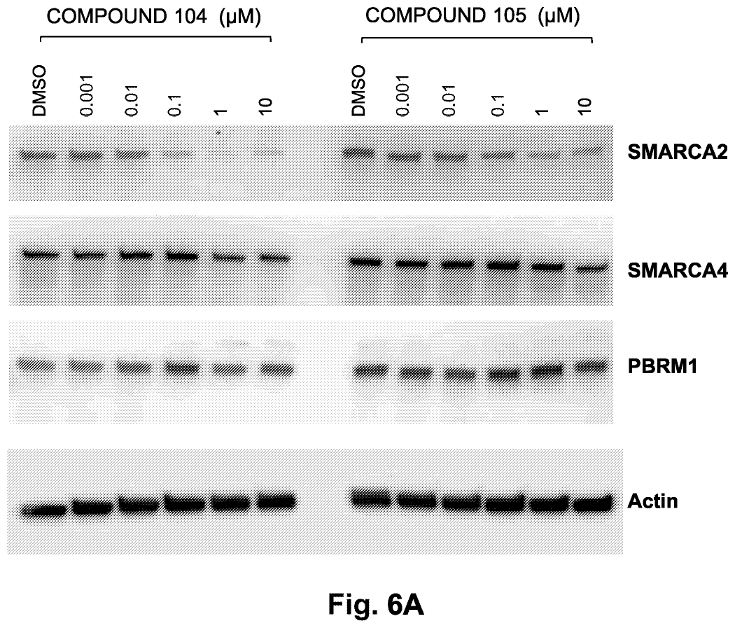

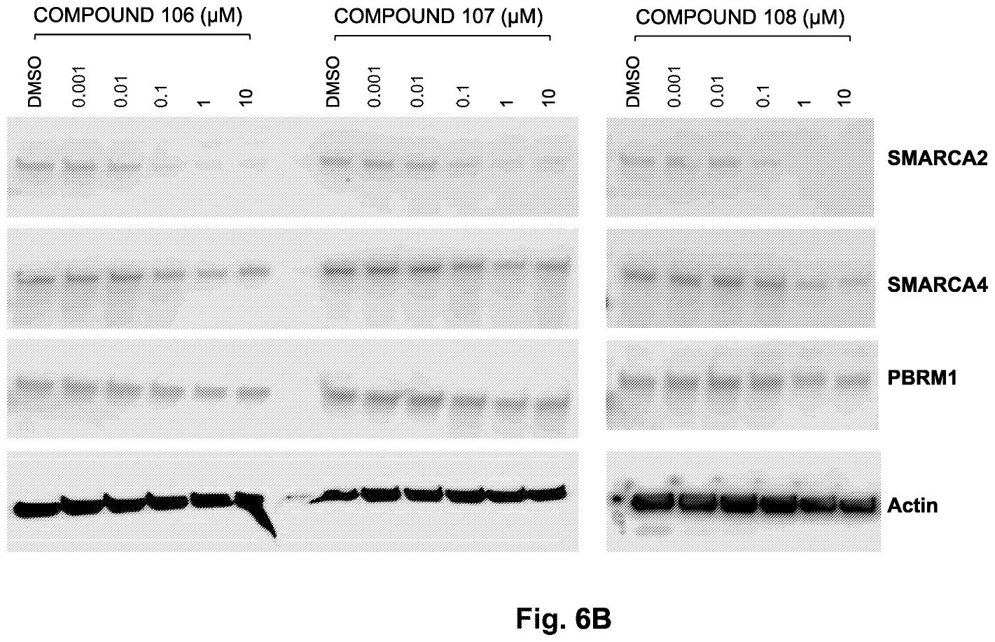

Cell lines with wild-type SMARCA4 (H1975) were treated with DMSO or 0.001, 0.01, 0.1, 1, or 10 μm of Compounds 104, 105, 106, 107, or 108 and analyzed by western blot for SMARCA2, SMARCA4, and PBRM1 expression (FIGS. 6A and 6B). Compounds 104-108 efficiently degraded SMARCA2 but not SMARCA4 or PBRM1 (FIGS. 6A and B).

### Example 14—Immunohistochemistry to Visualize SMARCA2 Degradation in Vivo

NCR Nude mice were injected with vehicle or SMARCA2 degrader Compound 101 (200 mg/kg) once per day for four consecutive days intraperitoneally. Tumor tissues were harvested 24 hours after the last dose and fixed in formalin for 24 hours, paraffin embedded, sectioned and stained according to standard procedures. Briefly, endogenous peroxidases were inactivated by 3% hydrogen peroxide. Non-specific signals were blocked using 3% BSA, 10% goat serum in 0.1% Triton X-100. After antigen retrieval in citrate buffer, slides were stained overnight at 4° C. using SMARCA2 antibodies (Cell Signaling, D9E8B1:2000). After overnight incubation, the slides were washed and incubated with secondary antibody (HRP-polymers, Biocare Medical) for 30 min at room temperature. The slides were washed three times and stained with DAB substrate (ThermoFisher Scientific). The slides were then counterstained with haematoxylin and mounted with mounting medium. The slides were imaged by Vectra microscope and the signal intensity and percentage of cells positive for SMARCA2 was quantified using Vectra 3.0.7 software.

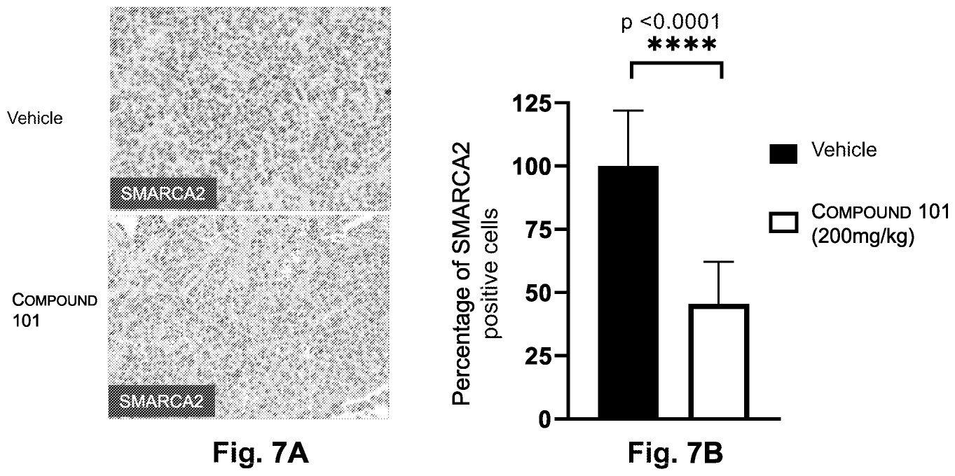

A significant reduction in SMARCA2 was observed in Compound 101 treated NCR nude mice compared to vehicle controls (FIG. 7A) with a more than 50% reduction in the percentage of SMARCA2 positive cells following Compound 101 treatment (FIG. 7B).

### Example 15—In Vivo Anti-Tumor Efficacy Study in Female NCR Nude Mice Using a HCC515 Lung Cancer Xenograft Model

About 1×10⁷ HCC515 cells were injected into the flanks of female NCR Nude Mice. Tumor take was monitored visually and by palpation bi-weekly. Once tumors reached an average volume of 100 mm³ mice were randomized into control and SMARCA2 degrader groups. Treatment cohorts were as follows: Vehicle was 15% Solutol in water together with 12.5 mg/kg of ritonavir as metabolic stabilizer. In the vehicle group, ten animals were injected with vehicle intraperitoneally. In the treatment group, ten animals were injected intraperitoneally once daily with 12.5 mg/kg of the SMARCA2 degrader Compound 101 in 15% Solutol in water together with 12.5 mg/kg of ritonavir as metabolic stabilizer.

Tumor diameter and volume were calculated based on caliper measurements of tumor length and height using the formula tumor volume=(length×width²)/2. Tumor volume and body weight were measured three times a week.

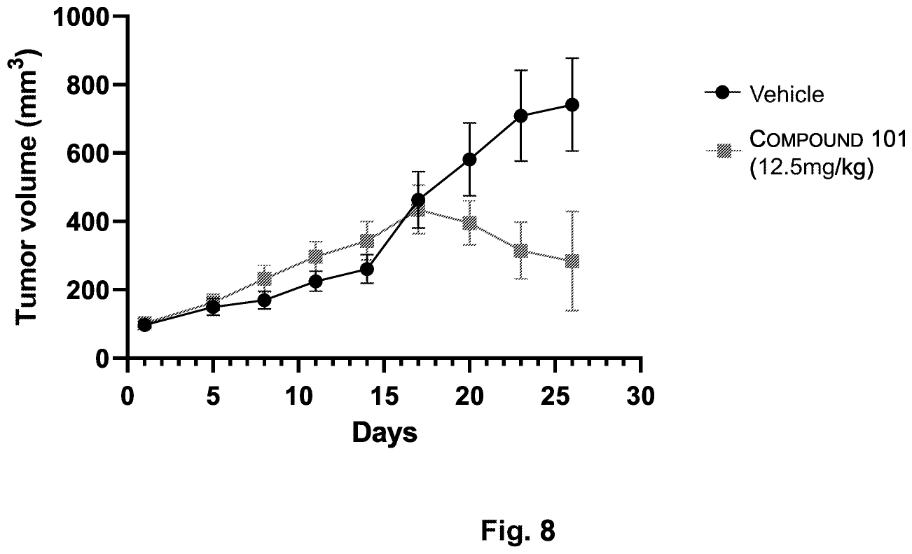

A substantial reduction in tumor volume in Compound 101 treated mice was observed with vehicle control mice demonstrating the efficacy of a SMARCA2 degrader to block tumor growth in vivo (FIG. 8).

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. A compound of Formula I:
D-L-S
or a pharmaceutically acceptable salt thereof,
wherein:
D is a ubiquitin ligase binding moiety,
L is a linker comprising a fluoro-phenyl or difluoro-phenyl substituted with one or more groups selected from the group consisting of a piperazine group, a piperidine group, methoxypropyl and a methoxyethoxy group, and
S is a SMARCA2 binding moiety.

2. A compound of Formula II: 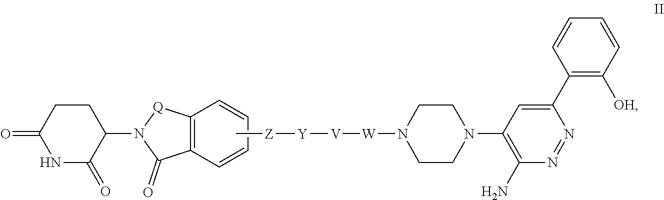
or a pharmaceutically acceptable salt or solvate thereof, wherein:
Q is selected from the group consisting of —CH₂— and —C(═O)—;
W is —CH₂—;
V is: 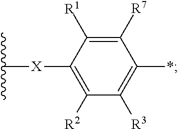
wherein the bond marked with an “*” is attached to W;
R¹, R², R³, and R⁴ are independently selected from the group consisting of hydrogen and fluoro;
X is selected from the group consisting of —O—, N(R^{5a})—, 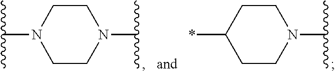 [SMILES: *N1CCN(*)CC1 ; *C1CCN(*)CC1]
wherein the bond marked with an “*” is attached to Y; or
X is a bond;
R^{5a} is selected from the group consisting of hydrogen and C₁-C₄ alkyl;
Y is selected from the group consisting of C₁-C₈ alkylenyl and 3- to 8-membered heteroalkylenyl; or
Y is a bond; and
Z is selected from the group consisting of —O—, —N(R^{5b})—, 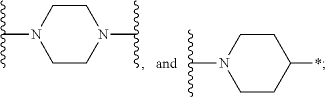
wherein the bond marked with an “*” is attached to Y; or
Z is a bond; and
R^{5b} is selected from the group consisting of hydrogen and C₁-C₄ alkyl.

3. The compound of claim 2 of Formula III: 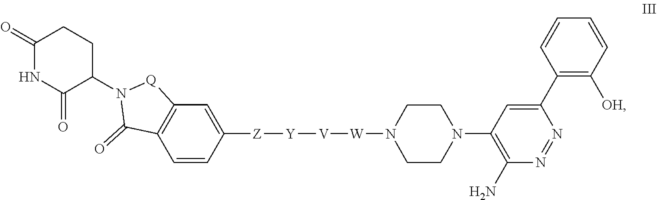
or a pharmaceutically acceptable salt or solvate thereof.

4. The compound of claim 2 of Formula IV: 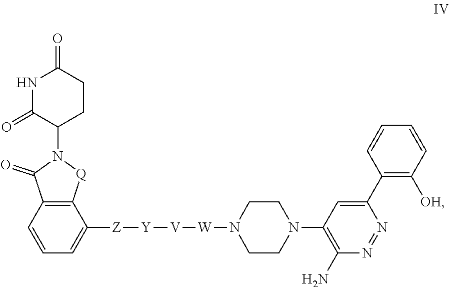
or a pharmaceutically acceptable salt or solvate thereof.

5. The compound of any one of claims 2-4, or a pharmaceutically acceptable salt or solvate thereof, wherein Q is —C(═O)—.

6. The compound of any one of claims 2-5, or a pharmaceutically acceptable salt or solvate thereof, wherein R¹ is fluoro, and R², R³, and R⁴ are hydrogen.

7. The compound of any one of claims 2-5, or a pharmaceutically acceptable salt or solvate thereof, wherein R⁴ is fluoro, and R¹, R², and R³ are hydrogen.

8. The compound of any one of claims 2-5 or a pharmaceutically acceptable salt or solvate thereof, wherein R¹ and R² are fluoro, and R³ and R⁴ are hydrogen.

9. The compound of any one of claims 2-5 or a pharmaceutically acceptable salt or solvate thereof, wherein R¹ and R⁴ are fluoro, and R² and R³ are hydrogen.

10. The compound of any one of claims 2-5, or a pharmaceutically acceptable salt or solvate thereof, wherein R¹ and R³ are fluoro, and R² and R⁴ are hydrogen.

11. The compound of any one of claims 2-5, or a pharmaceutically acceptable salt or solvate thereof, wherein R³ and R⁴ are fluoro, and R¹ and R² are hydrogen.

12. The compound of any one of claims 2-11, or a pharmaceutically acceptable salt or solvate thereof, wherein X is a bond.

13. The compound of any one of claims 2-11, or a pharmaceutically acceptable salt or solvate thereof, wherein X is —O—.

14. The compound of any one of claims 2-13, or a pharmaceutically acceptable salt or solvate thereof, wherein Y is C₁-C₈ alkylenyl.

15. The compound of claim 14, or a pharmaceutically acceptable salt or solvate thereof, wherein Y is selected from the group consisting of —CH₂—, —CH₂CH₂—, —CH₂CH₂CH₂— and —CH₂CH₂CH₂CH₂—.

16. The compound of any one of claims 2-13, or a pharmaceutically acceptable salt or solvate thereof, wherein Y is 3- to 8-membered heteroalkylenyl.

17. The compound of claim 16, or a pharmaceutically acceptable salt or solvate thereof, wherein Y is selected from the group consisting of —CH₂CH₂OCH₂CH₂— and —CH₂CH₂OCH₂CH₂CH₂—.

18. The compound of any one of claims 2-17, or a pharmaceutically acceptable salt or solvate thereof, wherein Z is of —O—.

19. The compound of any one of claims 2-17, or a pharmaceutically acceptable salt or solvate thereof, wherein Z is of —N(R^{5b})—.

20. The compound of any one of claims 2-19, wherein —Z—Y—V—W— is selected from the group consisting of: 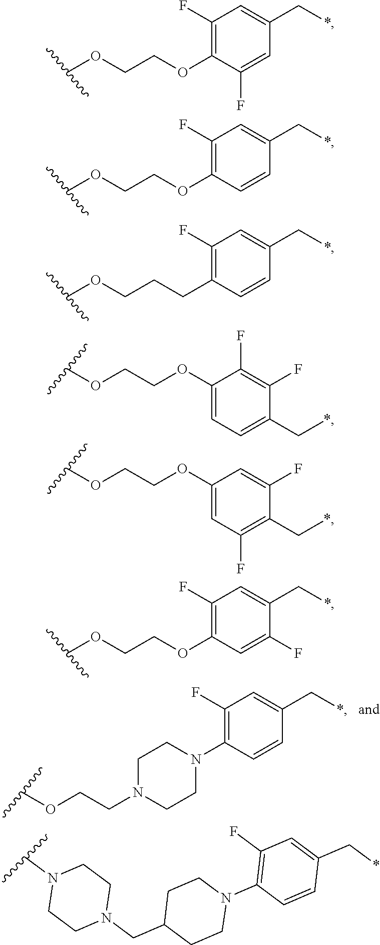
wherein the bond marked with an “*” is attached to the SMARCA2 binding moiety.

21. The compound of any one of claims 1-20 wherein the compound is selected from the group consisting of: 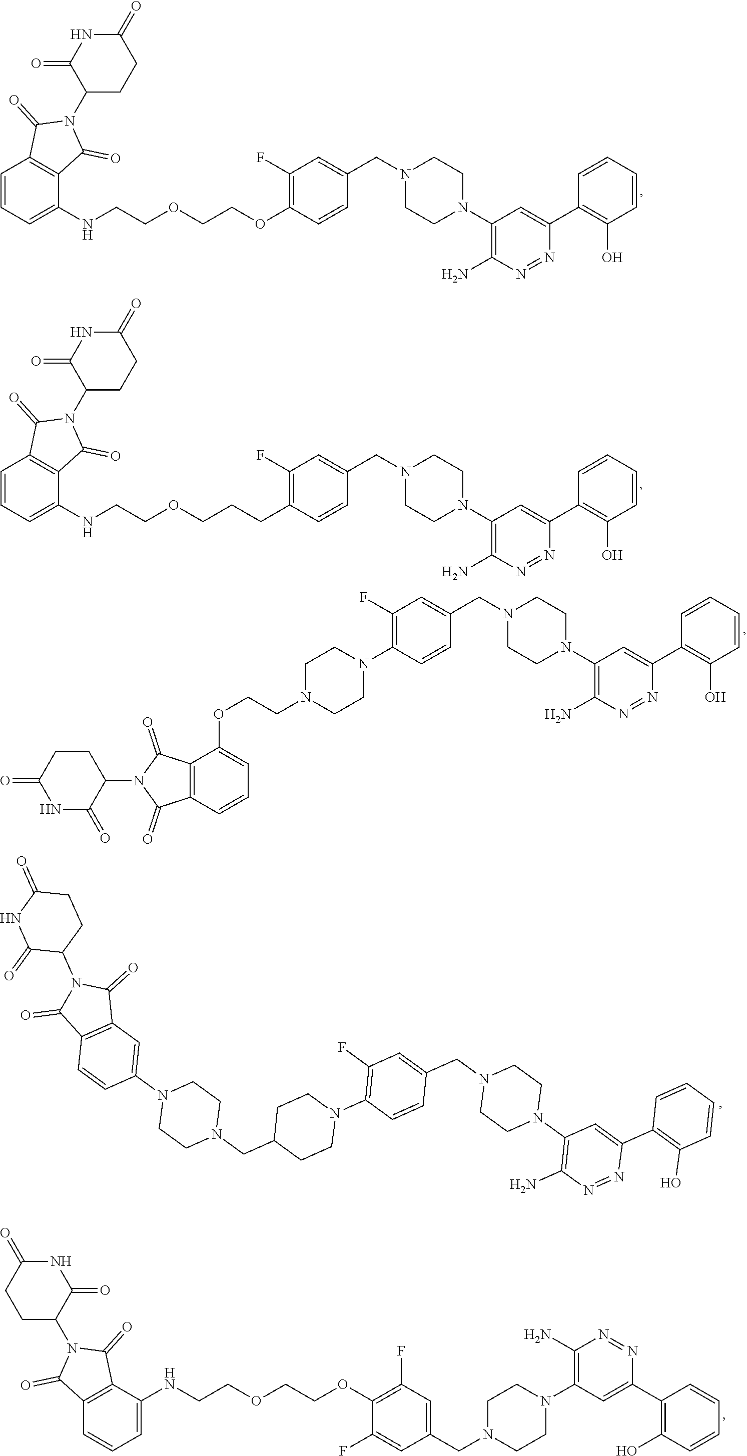 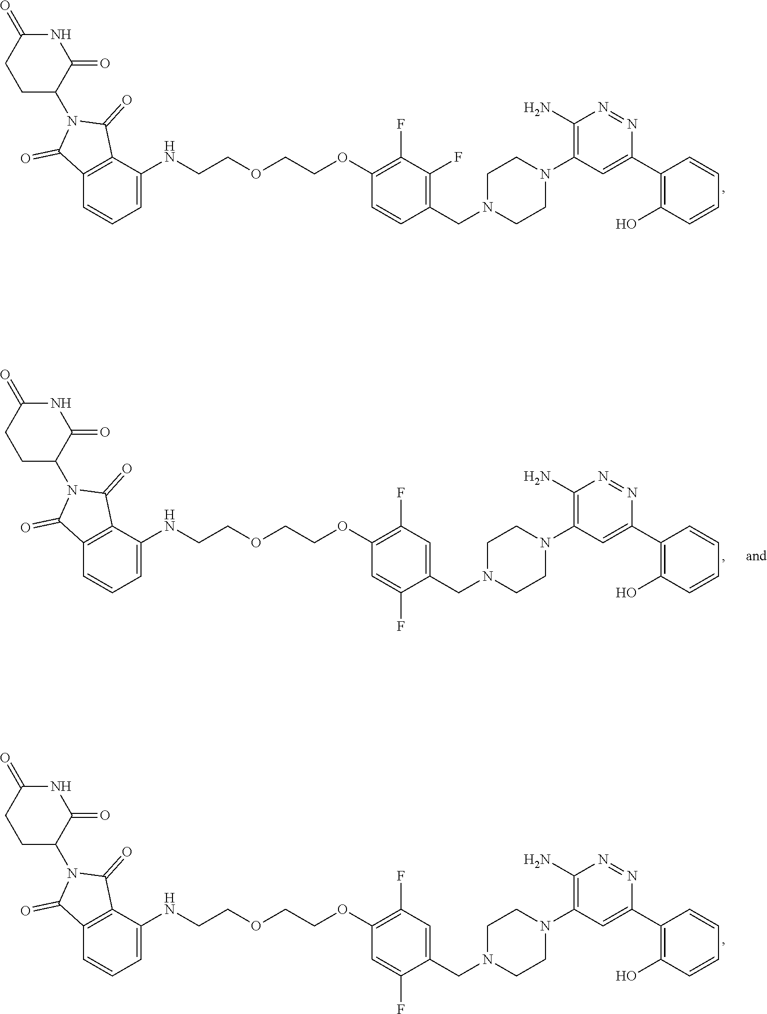 [SMILES: Nc1nnc(-c2ccccc2O)cc1N1CCN(Cc2ccc(OCCOCCNc3cccc4c3C(=O)N(C3CCC(=O)NC3=O)C4=O)c(F)c2F)CC1 ; Nc1nnc(-c2ccccc2O)cc1N1CCN(Cc2cc(F)c(OCCOCCNc3cccc4c3C(=O)N(C3CCC(=O)NC3=O)C4=O)cc2F)CC1 ; Nc1nnc(-c2ccccc2O)cc1N1CCN(Cc2cc(F)c(OCCOCCNc3cccc4c3C(=O)N(C3CCC(=O)NC3=O)C4=O)cc2F)CC1]
or a pharmaceutically acceptable salt or solvate thereof.

22. The compound of any one of claims 1-21, wherein the compound has a DC₅₀ (concentration at which 50% of the target protein is degraded) for SMARCA2 of about 0.1 nM to about 300 nM, about 1 nM to about 200 nM, about 2 nM to about 150 nM, about 5 nM to about 120 nM, about 10 nM to about 100 nM, about 15 nM to about 80 nM, about 20 nM to about 70 nM, or about 30 nM to about 60 nM.

23. The compound of any one of claims 1-21, wherein the compound has a DC₅₀ for SMARCA2 of less than about 300 nM, less than about 200 nM, less than about 150 nM, less than about 120 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, or less than about 2 nM.

24. The compound of any one of claims 1-21, wherein the compound has a Dₘₐₓ (maximum degradation as percentage of DMSO control) for SMARCA2 of about 60% to about 100%, about 65% to about 99%, about 70% to about 95%, or about 75% to about 90%.

25. The compound of any one of claims 1-21, wherein the compound has a Dₘₐₓ for SMARCA2 of about 60%, 65%, 67%, 70%, 72%, 75%, 77%, 80%, 82%, 85%, 87%, 90%, 92%, 95%, 97%, 98%, 99%, or 100%.

26. The compound of any one of claims 1-21, wherein the compound has a DC₅₀ for SMARCA4 of about 500 nM to about 100 μM, about 700 nM to about 90 μM, about 800 nM to about 80 μM, about 900 nM to about 70 μM, about 1 μM to about 60 μM, about 2 M to about 50 μM, about 5 μM to about 25 μM, or about 10 μM to about 20 μM.

27. The compound of any one of claims 1-21, wherein the compound has a DC₅₀ for SMARCA4 of more than about 500 nM, more than about 700 nM, more than about 1 μM, more than about 2 μM, more than about 5 μM, more than about 7 μM, or more than about 10 μM.

28. The compound of any one of claims 1-21, wherein the compound has a Dₘₐₓ for SMARCA4 of about 10% to about 80%, about 15% to about 70%, about 20% to about 60%, or about 25% to about 50%.

29. The compound of any one of claims 1-21, wherein the compound has a Dₘₐₓ for SMARCA4 of less than about 80%, less than about 70%, less than about 60%, less than about 50%, less than about 40%, less than about 25% or less than about 20%.

30. The compound of any one of claims 1-21, wherein the compound has an IC₅₀ (concentration at which 50% of cellular proliferation is inhibited) of about 10 nM to about 50 μM, about 50 nM to about 20 μM, about 100 nM to about 10 μM, about 200 nM to about 8 μM, about 300 nM to about 5 μM, about 400 nM to about 2 μM, or about 500 nM to about 1 μM.

31. The compound of any one of claims 1-21, wherein the compound has an IC₅₀ in wild-type SMARCA4 cells of about 900 nM to about 50 μM, about 1 M to about 40 μM, about 2 μM to about 30 μM, about 5 μM to about 25 μM, or about 10 μM to about 20 μM.

32. The compound of any one of claims 1-21, wherein the compound has an IC₅₀ in wild-type SMARCA4 cells of more than about 900 nM, more than about 1 μM, more than about 2 μM, more than about 3 μM, more than about 5 μM, more than about 7 μM, or more than about 10 μM.

33. The compound of any one of claims 1-21, wherein the compound has an IC₅₀ in mutant SMARCA4 cells of about 50 nM to about 10 μM, about 75 nM to about 5 μM, about 100 nM to about 1 μM, about 125 nM to about 900 nM, about 150 nM to about 800 nM, about 175 nM to about 700 nM, about 200 nM to about 600 nM, or about 250 nM to about 500 nM.

34. The compound of any one of claims 1-21, wherein the compound has an IC₅₀ in mutant SMARCA4 cells of less than about 10 μM, less than about 5 μm, less than about 2 μM, less than about 1 μM, less than about 900 nM, less than about 800 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, or less than about 2 nM, or less than about 1 nM.

35. The compound of any one of claims 1-34, wherein the compound competitively inhibits binding of pomalidomide to cereblon.

36. The compound of any one of claims 1-35, wherein the compound competitively inhibits binding of a SMARCA2 inhibitor to SMARCA2.

37. The compound of any one of claims 1-36, wherein the compound inhibits proliferation of SMARCA4 mutant cancer cells.

38. The compound of any one of claims 1-37, wherein the compound does not inhibit proliferation of SMARCA4 wild-type cells.

39. A pharmaceutical composition comprising the compound of any one of claims 1-38, or a pharmaceutically acceptable salt or solvate thereof, and a pharmaceutically acceptable adjuvant, carrier, or vehicle.

40. The pharmaceutical composition of claim 39, further comprising a surfactant.

41. The pharmaceutical composition of claim 39 or 40, further comprising an agent with CYP3A4 inhibitory activity.

42. The pharmaceutical composition of claim 41, wherein the compound is Compound 101, Compound 104, Compound 105, Compound 106, Compound 107, or Compound 108.

43. The pharmaceutical composition of claim 39 or 41, wherein the composition does not comprise an agent with CYP3A4 inhibitory activity.

44. The pharmaceutical composition of claim 43, wherein the compound is Compound 102 or Compound 103.

45. The pharmaceutical composition of any one of claims 39-44, further comprising an additional pharmaceutically active agent.

46. The pharmaceutical composition of claim 45, wherein the additional pharmaceutically active agent is a hormone analogue or antihormone, an aromatase inhibitor, a growth factor inhibitor, an antimetabolite, an antitumor antibiotic, a platinum derivative, an antimitotic agent, an angiogenesis inhibitor, a topoisomerase inhibitor, a serine/threonine kinase inhibitor, a tyrosine kinase inhibitor, a PARP inhibitor, a tubulin inhibitor, a DNA synthesis inhibitor a protein-protein interaction inhibitor, a MEK/ERK inhibitor, a TRAIL inhibitor, a BCR-ABL inhibitor, a HDAC inhibitor, a radiopharmaceutical, an immune checkpoint inhibitor, an ADCC enhancer, a T cell engager, a chemotherapeutic agent, or a tumor vaccine.

47. A method of inducing degradation of a SMARCA2 in a subject in need thereof, comprising administering to the subject a therapeutically effective amount of the pharmaceutical composition of any one of claims 39-46.

48. The method of claim 47, wherein the subject suffers from a SMARCA4 deficient malignancy.

49. The method of claim 48, wherein the SMARCA4 deficient malignancy comprises a SMARCA4 inactivating mutation or a SMARCA4 deletion.

50. The method of claim 49, wherein the SMARCA4 deletion is a SMARCA4 gene truncation.

51. The method of any one of claims 47-50, wherein the SMARCA4 deficient malignancy is a lung cancer, a liver cancer, a bladder cancer, a breast cancer or a colon cancer.

52. The method of claim 51, wherein the lung cancer is non-small cell lung cancer.

53. A method of treating a SMARCA4 deficient malignancy in a subject in need thereof, comprising administering to the subject a therapeutically effective amount of a pharmaceutical composition of any one of claims 39-46.

54. The method of claim 53, wherein the SMARCA4 deficient malignancy comprises a SMARCA4 inactivating mutation or a SMARCA4 deletion.

55. The method of claim 54, wherein the SMARCA4 deletion is a SMARCA4 gene truncation.

56. The method of any one of claims 53-55, wherein the SMARCA4 deficient malignancy is a lung cancer, a liver cancer, a bladder cancer, a breast cancer or a colon cancer.

57. The method of claim 56, wherein the lung cancer is non-small cell lung cancer.

58. A method of treating a tumor in a subject in need thereof, comprising administering to the subject a therapeutically effective amount of the pharmaceutical composition of any one of claims 39-46.

59. The method of claim 58, wherein the tumor comprises a SMARCA4 inactivating mutation or deletion.

60. The method of claim 58 or 59, wherein the tumor is a lung cancer, a liver cancer, a bladder cancer, a breast cancer or a colon cancer.

61. The method of claim 60, wherein the lung cancer is non-small cell lung cancer.

62. The method of any one of claims 47-61, wherein the method further comprises administering a hormone analogue or antihormone, an aromatase inhibitor, a growth factor inhibitor, an antimetabolite, an antitumor antibiotic, a platinum derivative, an antimitotic agent, an angiogenesis inhibitor, a topoisomerase inhibitor, a serine/threonine kinase inhibitor, a tyrosine kinase inhibitor, a PARP inhibitor, a tubulin inhibitor, a DNA synthesis inhibitor a protein-protein interaction inhibitor, a MEK/ERK inhibitor, a TRAIL inhibitor, a BCR-ABL inhibitor, a HDAC inhibitor, a radiopharmaceutical, an immune checkpoint inhibitor, an ADCC enhancer, a T cell engager, a chemotherapeutic agent, or a tumor vaccine.

63. A method of treating a condition or disease selected from a chronic autoimmune disorder, an inflammatory condition, a proliferative disorder, a sepsis, or a viral infection, comprising administering to a subject in need thereof a therapeutically effective amount of the pharmaceutical composition of any one of claims 39-46.

64. A heterobifunctional compound comprising two binding moieties connected by a linker, wherein the linker is selected from the group consisting of: 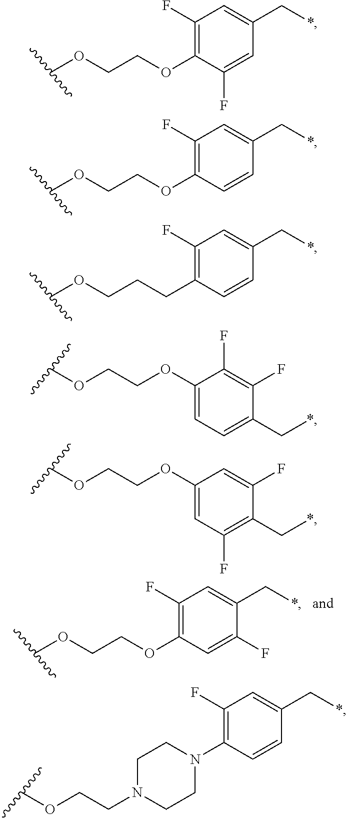
wherein the bond marked with an “*” is attached to one of the two binding moities.

65. The heterobifunctional compound of claim 64, wherein the linker is selected from 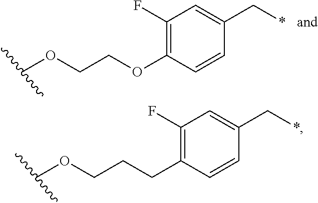
wherein the bond marked with an “*” is attached to one of the two binding moieties.
